SEQUENCE LISTING (40 sequences)

SEQ ID NO: 1 - DNA, 8083 bp, Arabidopsis thaliana
  source 1..8083: mol_type = genomic DNA
ctgtcttaaatggacatttgtagtaacaaaaccgaccatcagcaaattaacgttaacgttgctctaatcgtgtgtatttttctaatgcttggtgaagcacatgcgaccatttgttgtgtcagaacaaaaggaagattccacatttagaatttagattataatcccacatgcctattatttaaacatatgtatactgtaataaattagtcccagaagagcatttcatcatactgtataattaatcaaatttaaaaaacatttgtgttgaagagaagatgactagattcacttgattttttttaatactgattggtcgtttgaccagtcaggatccagactgaaatattctcaaaaggtatgaactattttaatgaaatatttgagctgagaatttgcttatgggacattctatccttctaaagaaatcattagaaaatggagaaaaagttactagaaaaagagagggaaatggggcgagagtatgaagaaagggtcccatgtgaatctaataattgaaaagcaaatatgagatttattttagggttcttgttttagcgtccaaagcggtcgcagttgccgttcagcgacttttctctgtttatgaaataaaaacgctccacttgttcttttcttctttcaacccactttgttataactattaacaaatattgcttggtccaaattttattatactgaattattttagtttttttgtatcatcatcctcaatgttagttttgaaatcctaggtcagtaagtcaataacgaaaatgacaagtttaattttgtaattggaacttacatgtcacggtctttgtttacgatcattgccacgaattgtttagatacgggctttttttttctttccgtgttgatgattatcgtttcttcttacatatcttgatcaatcactaacatttacataacctacaattttgtaatataattgcaatactcgagatcttcaaatggtgatactgttaataaaattacgaacttttaatcatcatcaaaatctatagaaaaagatacataggaccaaaacaagcctaaaacaaagagttataatttacaaataaatgcatgtattttgttacttttgttaaggattttcatatcaatgcatgcataatgcattctctttttattttatttataatgcattcttttataatattcggatccaaaattatgaattaattccagattgccataaatgtctttctttgtttatgctgacttttttcttttcttaacttatatagtaagtaaaccagaaaggtggaagatacaaagcaatttcattgcattaataaacttcaaatgtattatccaataatcaaaatttatctagtaattcacaaaaaaaaaaaaatctgtctatcattcaaaatttcaccaatttggataaatatctagaaaattttaaaatacaattcaataacactattaacatatcaatcaatatataacattgcaaaagtttacttgatcattagtatattcattttaaggttaaaagatctttaagaaaaggaaattcaaattatattgttggaaaaaaatacaaatcaaagacagcaaaaaaagtaacaaaaaaaaaaggtagaagaaactgaaacgcggaaaggaggcaaaatcttctcgtcgtcgttgtcgccgtcttcagagctacaaacgaaaaaactcgcttccgtttcgatttctccattgttattgtttcttcagtgaagcttttttcttcgagaaatttctaagatctaccacatgctactatgagcctccgccaccgcaccgtcccgccgcaaaccggacggccgttggcggcggaagctgtcggaatcgaagaggagccgtacaatatcattcccgttaacaatctcctcgccgaccatccttcactccgttttcccgaggttcgtgccgccgctgctgctcttaaaaccgttggagaccttcgtcgtccgccgtatgttcaatggcgttctcactacgatctcctcgactggctcgccttgttcttcggtttccagaaagataacgttcgtaaccagcgtgagcatatggtgcttcatctcgcaaatgctcagatgcgtctctctccgccgccggataatattgattctctcgattccgcggttgttcgtcggtttcgtcggaaacttctcgctaactactctagctggtgttcgtatttggggaaaaaatcaaatatctggatctcagatcggaaccctgattcgagacgagagcttctctatgttggactctatcttctcatttggggagaggctgcgaatcttcggttcatgcctgaatgtatctgttacatcttccataacatggcctctgagctcaacaaaatcttagaggattgcctcgatgagaacaccggccaaccttacttgccttctctctcaggcgaaaacgctttcttaaccggcgtcgttaaacctatttacgatactatccaagctgagattgatgagagcaagaacggtacagttgcgcattgtaagtggaggaactacgacgatatcaatgagtacttctggactgatcggtgtttcagcaaattgaaatggccgcttgatttgggaagcaatttctttaagagtagaggcaaaagtgtagggaaaactggtttcgtggagcgcaggacgttcttctacctttacaggagttttgatcgactttgggtgatgctagctttgttccttcaagccgccattatagtagcttgggaggaaaagccagatacctcgtcggtaacaaggcagctgtggaatgctctgaaggcaagagatgttcaggtgagactattgaccgtgttcttgacatggagtggtatgcgactcttgcaggctgtgctggacgcggcttcacaatatcccctcgtttccagagagaccaaaaggcattttttcagaatgctgatgaaggttatagctgccgcagtttggattgtagctttcactgtcctctacactaacatctggaagcagaagaggcaagacaggcagtggtccaatgccgcgacgactaagatataccaattcctttacgctgtgggggccttcttggtgcccgaaatcctggctttggctttgtttattatcccatggatgagaaacttcctggaagagaccaattggaaaatattctttgctctaacttggtggtttcaaggcaaaagctttgtgggtcgaggtttgagagagggtttagtggacaacatcaagtactcgactttctggatctttgtcctagctacaaagtttacatttagttacttcctgcaggttaagccaatgattaaaccctcaaagctgctatggaacttaaaggatgtcgattatgagtggcatcagttttatggagacagcaataggttttctgtcgcattgttatggttgccagttgtgttgatatatctgatggatatccaaatttggtacgcaatctattcttcgattgttggtgctgttgttgggctgtttgatcatctgggggagatcagggacatgggacagctgaggctaaggtttcaattctttgctagtgctattcaattcaacctaatgcctgaggaacaactcctgaatgctagaggctttggtaacaagttcaaggacggcattcataggtagtccgtggaagcatgctactaatattcctaaataattttctgtacaacgcttgacttgactgtacaagctgtgaaattttacttttgttaacgcagatgctgcatataattaaatttttcaatattgtaataacttgaggttgtgtactgtatgcagattgaagctaaggtatggatttgggaggccgtttaagaaacttgagtcgaatcaggtcgaggccaacaagtttgcgttgatctggaacgaaatcatcttagctttcagagaagaggatatagtttctgatcgtgaagtagagctactggagctgccaaagaattcctgggatgtgacggttattcgctggccgtgtttcttgttgtgcaatgagcttttgcttgcactgagccaggccagagagctgatagacgcacctgataaatggctgtggcacaaaatatgcaagaatgaatacaggcgttgtgctgtagttgaggcatatgacagcatcaaacatctattgctctcaatcatcaaagttgacactgaagaacattcgataattacggtcttctttcagataattaatcagtccattcagtcagagcagttcaccaagacctttagagtggacctgctgccaaaaatttatgaaacactgcagaaattggttgggctggtaaatgatgaggaaacagatagtgggcgggtggtgaatgttctgcagtctctttatgagattgcaactcgacagttctttatagagaagaagacaactgaacagctatctaatgaaggtttaactcctcgagacccagcctcaaagttgctgtttcaaaatgctattaggcttcctgatgcaagcaatgaagacttctaccggcaggttaggcgtttacacacgattctcacctctagggactctatgcacagcgtccctgtgaatctagaggcgagacggcggattgctttcttcagtaattcgcttttcatgaacatgcctcatgcccctcaggttgagaaaatgatggcgttcagtgttctgactccatattacagtgaggaagttgtatacagcaaagaacagctccgaaatgagactgaggatgggatttccaccctatactacctgcagacaatttatgctgatgaatggaaaaatttcaaggaacggatgcatagggaaggaatcaagacagatagtgagttgtggacaaccaagctgagagacctcaggctttgggcttcctacagaggtcagacattggcacgtacagttcgtgggatgatgtactactaccgggctcttaagatgctcgcttttcttgactctgcgtctgaaatggacattcgggagggtgctcaggagcttggttcagtgaggaatttgcagggagaactgggtggtcaatctgatgggtttgtctctgaaaacgaccgatcttccttaagcagagcaagtagttccgtgagtacgctgtataaaggccatgagtatgggactgcattgatgaaattcacatatgttgtggcgtgtcagatctacgggtctcaaaaagcaaagaaagagcctcaggcagaggaaattctgtatctgatgaagcagaacgaagctctccgtattgcatatgtggatgaggtgcctgcgggaagaggagagactgattattactccgttctggtgaaatacgatcaccagttggagaaggaagtggaaatattccgtgtgaagctacctggtccagtgaagctgggcgagggaaagccagagaaccagaatcatgcaatgatctttacccgtggtgatgctgttcagaccattgatatgaaccaagacagttattttgaggaagctctcaagatgagaaatttgctccaggagtacaaccattatcatggtatcagaaaaccaactattcttggtgtcagggagcatatcttcacgggatcagtctcgtcactggcgtggttcatgtctgctcaggagacaagttttgtcactcttggtcagcgtgttcttgcaaacccactgaaggtcagaatgcattatggccaccctgatgtatttgacagattctggttcttgagtcgaggcggcatcagtaaggcttccagagttataaatatcagtgaggacatctttgccgggtttaactgcacgttaagggggggaaacgtcacccaccacgagtacattcaggttgggaagggtccacaatttggattatttctaactaactatactgctacaacgtttttttaacgtttttaacgtttattaattatgcaatctacttttgttataattatgtaatttaacgttttttaatcttctaaattcaaaaaatttgagtaacctttgtctttatgcatttttcaggtcgggaagggacgggatgttggattgaatcagatatcaatgtttgaggctaaggtagccagtgggaacggagagcaggttctcagccgagatgtgtaccggctcgggcacaggcttgatttcttcagaatgttatcatttttctacacaactgtagggtttttcttcaacacaatgatggtcattcttactgtttacgctttcctctggggacgggtttatctggctctcagcggggttgagaagtccgctctagcagacagtacggacaccaacgccgcgcttggggtgatcctgaaccagcagttcatcattcagctcggtctgttcactgccctgccaatgattgttgaatggtctctcgaggagggtttccttctagcgatatggaatttcattcgaatgcagattcagctttcagctgtcttctacacattctcaatggggaccagagctcactatttcggtcgaactattctccatggtggggccaagtatagagccactggacgtggatttgttgtcgagcacaagggattcactgagaactaccgactgtatgcacgcagtcactttgtgaaggccatcgagcttgggctgatcctcatagtctacgcttcgcacagtccgattgccaaagactcgttgatttacatagccatgactatcaccagctggtttcttgtgatttcatggataatggccccatttgtgtttaacccatcaggattcgactggcttaagacagtctatgactttgaagacttcatgaactggatctggtaccaaggcagaatctcaacgaaatctgaacaaagctgggaaaaatggtggtacgaggaacaggaccacctgagaaacaccgggaaggcaggattatttgtggagatcatcttggtcctccggtttttcttcttccagtatgggattgtataccagcttaaaattgcaaacggatccaccagcctttttgtctacttgttctcatggatatacatctttgctatatttgtgctcttcctagtcatccaatacgcccgtgacaagtactcggcaaaagctcacatacggtacaggcttgtccaattcctcctgatcgtgcttgctatactggtgattgttgctttgctcgagttcacgcatttcagcttcatcgatatcttcacaagccttcttgcattcatcccaactggctggggaattctgctgatcgcacagactcaaaggaagtggctgaagaattacactattttctggaatgctgttgtctctgttgctcgcatgtatgacatattgtttgggatactcataatggttccagtagcgttcttgtcatggatgcctggattccagtcaatgcaaacgaggatattattcaatgaagcttttagcagaggacttcgcatcatgcagattgtcactgggaagaaatcaaaaggcgatgtctaagtttaaaaaacggtaaagctccttgttctcaacaccttatgttatgatcgtttaaatcctggatttcacaccaatgcgggctttaaatttgtgtaggtcttaagaagtaaatggtagttcaaatcctattggtatgtggcgaaggaatcagttggaggttagttttcccgaaacaaccgaattcgaagttttgtttcgtctaaagaaaaactcagatgctgatgatttatctttgtattttaaacaggtttttggagagtttggttggatgaggaatcgggaagttggtttgattcggttagatgggtttagggagatatttgattgtcagtgtgtgtggagggaactctgattcttgtatggtttttgttctaaaggtacagcaatttgtgtagtgaggctttgtgtatttgttctccttctctcattatagagctttagagcatttttagtttatattcagattgttatctaatgtcatctcgcagagcttttgttcacatttcacatcttttcttctccttcttagtagagatcagtttcagattagatacttgtccatattccactactctcgtctattatcggtttctgcttgtcaatttctgggtccaaaattgaaata

SEQ ID NO: 2 - DNA, 10394 bp, Brassica napus
  source 1..10394: mol_type = genomic DNA
cttccttctttgccgtctctcttgaagctaagtacaccgatatggccgcctttaagtatgttaaatgcaaacacaaatcgattatgttcatctcataagttatgtgaatatttttagctagtaattgggtataacatgttttgtaggtattttgtgatcgcaaacgctgtcgtgagtgtttacagctttctagttctgtttcttcctaaggagagtttactgtggaagttcgtcgtcgtcttggatttggtaataattgttatttggtttagtacttctcttcgttcattaattcatttgcattctatctctaagcatagtttttttctaaaggtaatattattgttgatatctagtagtttgatgtgttcagaggaataaaagcatttttattggtgaaatactgaaaaataataaaatgttgattattatagttcaaataaacaattgttttctagaagaacataaagtaatcacaatattatattttgcatgagagttttttcacgaaattctcatatttcctcttattatcatttatcgttgagatactttctaagaactctttaatggagatgttctagtaaattaatctttgaacgaaaagttaaaacatacactttcaaaaaaaaaaagttaaaacaaataaacttgaatgtaatgtgtcttagtcacacgcttattataggttgttaggtgacgactatatcagcatacatacgaattgtcattaacttttgacatttttggtgaatgtattggaggtgatgacaatgctactaacgtcaagcatatcagcggcgttagcggtggcgcaagtgggaaagaaaggaaacgcaaacgcaggttggcttccaatttgcggccaagttccaaagttttgcgatcaggtcaccggagctctcattgccggcttcgtcgcactcgtcctctacgtcttgttactcttatactctcttcactccgtcgtcgatccttttcttctccagaaatcttgaatctaacttcttttttcactttcagtatcggttaatatacgttttaatatctatactataacgtcgtttgtgtattgtatttatatgttttctgttttaggtgtcgagattgtcatagtatttcatttttatggactaattaacattatagactgaaccacctctcataaactcttactcataattaaaaaaaaaaatcaatgcttcatatatacatctctgtcaatcattggattcgtatttattatagtatcaacaaatttgtgatacaaacctaaacatgtgcaatatctttagctaacgaaatggatatagatacatcttccctagctatggaaatgagtatttgatcaaaacgtaattgcacttgttagttagtcatttctctcagtgtaacaaaacaccatttatgaaaaaacactgagcatgggcaagtttcgtcttcacctccgttaatcttttgcatcgtaaaaacaatcgatgttacaaaacatgttttgttttaacgttgtaaaaatatcaggcttgatttgaatgtcatgttatgccgactttactagtttgtgtaaccaactaagctattaaaatatgcgtaattacaaaactgtcagatttagtttcacaaaatctttgtaacttcgcggtttttgatatggaagttgctacaaaaactgacttgtgatattataatctttcggcaagaaaaatatataatactaatttcagaaagtattctgttgaattgttgatcaccaaatacacataaaccggcagatgttgacatgtttacgtgggatcactgggatagaagttttgcttctgtattagtatcgcgaaccggtttgtagtagcgaaaataacattattattttcagatttcatcatcgtcaaattcaacgttacatttgaacaggcttgtaaaaagtggacatgtggcatgagtaaaagataaagaaaattgtgagcctttttgttaatacgaaaaagccgaaaacgttattatttctatcatcggcaaatttaatgttaaatttgctccacatcattcagagggtatatgtatatttttctaatgcttcggtgaagcacatgcgaccatttgttgcgtcagaagtcagaacaaaaggaagattccatctaatggattataatcccacatgcccatttttaaaactagacaaaataacttataatagtcccataagagcattaagacaaactgaaccaaaaagaatcttaaatgctggtgaatctagtctcgaatgctgctgaacactcctgatattagaatttggcagaaaatattcatactaataacagtaaatacatttcaaaacaagaaaatagtagtagtaaataaaaacaattaatatgttattattgtttctcttagctgttaccattacattaacaatttacattcaatccttttaatgaaaaatattataaaatattaaaagaagttattagaaaaggagagggatatgggggggggggggggagagcatggaggggtcccatgtgtgtttcgaaaagcagcaaacatgactttgtaaagtcttgttttagctagagttccaaaaaaaaaagagttcaaaaaaaaagaaaaaaaaaagtcttgttttagcgtccaaagcggtctcactcgccgttcagctgttaaattttcttgaaataaatcgctccacttgttttttatttttgtttctttcaacccacttgtattaataacaataatgatattgctaatcttgaacaaatcgtggattgtcttttcttccagtgatatcatccttagtcaaaacatctcaaattaaataaatatatagttgtgaattaaaaaaaaaggtatagagtttataaaattagcatttgcgaatgtttttaaaagtttggcgtttgtatagatcactgtcagtgtcatgaatcattcggtgacagcaaatttgtatatgccaaaaaagagagttgatacttgccattctcgatattttgaattttttttcgtcgacatacaaactcaacattttcgaaatcttagattaaaaacaaaatggacaaaacgatacttattaatgaattatgtactagcgtctaaacattagtaacatagatataattataagaacaagaaacatatatgcacaaagcctcaaaccaaatttaaatagtgttacgaaacaagggatgtacttgttatgaaaaattaatgtataaccattcttagcattttttacctaacccttaaactttataaagaaaaatatgtaacatttcggtgtgtggtatataaaagtattttgaaaatcgatttgattatttctattattaaaatgtacttactttttttatcacacatttatttacaatttcagagttaaacatgattaagacaaagtagtgaaaatatagatgatctaccagaaaatcatttgctttctgagtgagatcaaaataaaggcaaaaataatgtgatgattgtatattaaacaattattttgaatttgaaaagaataactcatcgaatgaagcctatgaaccgagtaagcggtgaagcggttggaccgtcacaaaaattatgaccaacaagaaaataaaattgatattagtgaatgatatattttggaaagtaataaattggttccagatttcaatacatcaatgtttttctaatgctgacttgcttttcctctttttcttacatgataaggaaacgagagaggtcgaagaaacaaagaaacttcctaagcatttagcaacgcatcttcaaaaaatctagtatttaccaagtaaaatcatacaaaataaggaaaaaaagaagaatatatttcccaatttggttttcacccgaaaaaaaaactgtcttaacaaaatcttaatgcaccaaatcatataagaactttatttgaccattgtcattagttttttccttatggtaatggatcttcaaataatttaaattaaattaaaaaaaaaagaaaagagaaacaaaaacgcggaaaggagagcccaaatcgtctccgtctgtcattgtcgcagtctctaaaagagaaaaacaaatcgcaacgcttctctctctctccctatcgtttgaatcagtctctcaagatccaccaccaccacacatgctactatgagcctccgccaccgcaccgtcccctctcaacccggacggcccccggcggcgggcgcaatcgaggacgagccctacaacatcatccccgtcaacaacctcctcgccgaccacccctccctccgctaccccgaggtccgcgccgccgccgccgccctcaaaaccgtcggcgacctccgccgccccacctacgtccaatggcgcccccactacgacctcctcgactggctcgccctcttcttcggcttccagaaggacaacgtccgcaaccagcgcgagcacctcgtcctccacctcgccaacgcccagatgcgcctcaccccgccgccggacaacatcgattccctcgatcccgccgtcgtccgccgcttccgccgcaagctcctcggtaactactccagctggtgctcctacctcgggaggaagtccaacatctggatctcggatcggagccccgattcgcggcgggagcttctctacgtcggcctctacctcctcgtctggggcgaggcggccaatcttaggtttatgcctgagtgtatctgttacatcttccacaatatggcctcggagcttaacaagatcttagaggattgcctcgacgagagcacggggcagccgtattctcctaagatcacgggggagaatagtttcctaaacggcgtcgttaagcctatctacgacacgatcagagctgagattaatgagagcaagaacgggacggagccgcattgtaagtggaggaactacgatgatattaatgagtacttctggacggataggtgttttagtaaattgaaatggccgattgatttggggagcagtttcttcaagaacagcagaggtagcggagttgggaagacaggttttgtggagaggaggacgtttttctacctctacaggagctttgataggctttgggtgatgcttgctttgtttcttcaagctgctattatagttgcttgggaggagaagccgggtggagggtcggtgacgagtcagctctggaatgcgttgaagtcgacggatgttcaggtgaggcttttgactgtgttcttgacgtggagtgggatgaggttgttgcaggctgtgttggacgctggctcgcaacggtctcttatttctagagagaccaaacggctgtttttcagaatgttgatgaaggttgtggctgctacggtttggatagtagcgtttattgttctctacacgaacatctggaagcagaggaagcaagataggcagtggtccagagccgcgaatgataagatctatcagttcctttacgctgtggtggctttcttggttcctgagatcctggctttggctctgtttatagtcccgtggataaggaactttctggaagagacgaattggaagatattctttgctttgacttggtggttccaggggaaaagctttgtgggtcgaggtttgagagaggggttggtggacaacatcaagtactcgactttctggatctttgtccttgcaacgaagttcacgttcagctacttcctgcaggttaagccaatgattaaaccctcgaagctgctatggaatttgaaggaggtggattatgagtggcatcagttctttggcgagagcaataggttttctgtcttgttattgtggctgccagtggtgttgatatacctgatggatatccaaatttggtacgcgatctattcttcgattgttggtgctgttgttgggctgtttgatcatctgggggagatcagggacatgggacagcttaggctgaggtttcagttctttgctagcgctattcagttcaacctaatgcctgaggaacaactcctgaatgctagaggatttggtaacaagcttaaggacgccattcataggtaagtctattgaagcatgttactgatatttctatataatttactatatagagtttgtctttaaagtacaagctatagtattttagttttgttaaagcagattctccgcaatgaactcgactttttcacattgtaataacattgagtttgtgtactttatgcagattgaagctgaggtatggattggggcggccgtttaagaaactcgagtccaatcaggttgaggctaacaagtttgcgctgatctggaatgagataatcttagctttcagagaggaggatatagtctctgatcgagaagtagagctgctggagctgccaaagaattcctggaatgtgacagttatccgctggccgtgtttcctgttgtgcaacgaacttttgcttgcactgagccaggcgaaagagctggttgacgcacctgataaatggctgtggcacaagatatgcaagaatgagtacaggcggtgtgctgtggttgaggcatatgaaagcatcaaacatctgttgctctcaatcatcaaaattgacactgaagaacataaaattgttacaattttctttcagatgattgaggtgtctattcagggtgagcagttcaccaagaccttcaaagtggaccttttgccaaagatttatgagacactgcagaagttggttgggctgttgaatgatgagaaagtggatgttgggcgagtggtgaatggtctgcagtctatttatgagattgcaacacgacagttcttcctagaaaagaagacgactgaacagctatctactgaggggttaactcctcatgatccagcctcaaagttactgtttcagaatgctgttaggcttcccgatgcaagcaatgaagacttcttccggcaggttaggcggttacacacaattctcacttctagggactctatgcacagcgtccctgtgaatctagaggcgagacggcggattgccttcttcagcaattcgctcttcatgaacttgcctcatgcacctcaggtggagaaaatgttggcgttcagtgttatgactccatactacagcgaggaagttgtatacagcaaagaacagctccgaaatgagactgaggatgggatttcaaccttgtattacctgcagacgatttatgccgacgaatggaaaaattttaaggaacggatgcgtagggaaggtataaagacagatgttgagttgtggacaaccaagctgagagagctcaggctttgggcttcctacagaggtcagactttggcacgtacaattcgaggaatgatgtactattacagggctcttaagatgcttgcttttcttgactctgcgtctgaaatggacattcgggaggatgctcaggagcttggttcaatgaggagttcgcagggaaatcgattggatggggtggacgatgtaaatgacggatcttctctaagcagagcaactagctctgtgagcatgctgtataaaggccatgagcatgggactgcattgatgaaattcacatatgtcgtggcgtgccagatctatgggtctcaaaaagcgaagaaggagcctcaggcagaggaaattctgtatcttatgaagcaaaacgaagccctccgtattgcatatgtggatgaggtgcatgcgggcagggaagagactgagtattactccgttctggtgaaatacgatcacacgttggagaaggaagtggagatattccgtgtgaagctacctggtccggtgaagctgggtgagggaaagccagagaaccagaatcatgcaatgatctttacccgcggtgatgctgttcagaccatagatatgaaccaggataattattttgaggaggctctcaaaatgagaaatttactccaggagtttaggcattatcatgggatcagaaaaccaactattcttggtgtcagagagcacatcttcacgggttctgtctcgtctctggcttggttcatgtctgctcaggaaacaagtttcgtcactctgggtcagcgtgttctagccaacccgctgaaggtcagaatgcattatggtcaccctgatgtatttgacagattctggttcttgagtcgaggtggcatcagcaaagcttctagagttataaatatcagtgaggacatcttcgccgggtttaattgcacattgcggggcggtaacgtcacccaccacgagtatattcaggtagggaaatgttcatcatttggatattctaactaattttatacatcgacaacaatactataattccactttttttgttataacctttttgtgtgtgcatatgtattcaggttgggaagggtcgagatgttggattgaatcaaatatcaatgtttgaggctaaggtagccagtgggaatggagagcaggttcttagccgagatgtgtacaggttgggtcataggctcgatttcttcagaatgttatcatttttctacacaacggtggggtttttcttcaacacgatgatggtcattctcactgtctacgctttcctctggggccgggtttatcttgctctgagcggtgttgagaagtccgctctagcagacagcacagacaccaacgcagcgcttgctgtgatattgaaccagcaattcatcattcagcttggtctcttcacagctctgccaatgattgtggaatggtctctcgaggagggtttccttctcgcgatatggaacttcattcggatgcagattcagctttcctctgtcttctacacattctcaatggggaccagagctcactattttggccgaaccattctccatggtggagcaaagtacagagccactgggcgtggatttgttgtcgagcacaagagcttcactgagaattaccgtctatacgcacgcagtcactttgtgaaggccatcgagcttgggctgatcctcatagtctacgctacgcacagtcccatcgccaaagactcattgatctatatagctatgactctcaccagctggttcctcgtgatatcatggatactggccccttttgtgttcaacccgtcaggattcgactggcttaagacggtctacgacttcgaaggcttcatgaactggatctggtatcaaggcaggatctcaacgaagtccgaacagagctgggagatatggtggtatgaggaacaggaccacctgagaaccaccggtataccaggaagaatcgtggagataatcttggaccttcggtttttcttcttccagtacgggattgtataccagctcaaaatcgcaaacggatcaaccagcattctcgtctacttactctcatggatatacatcttcgcagtgtttgtgttcttcctagtaatccaatacgcccgtgacaagtactctgcgagaaaccacatacggtacaggctcgttcagttcctcctgatcgtgtttggtacactggtgattgttgctctcctcgagttcacgcatttcagcttcgtggatatcttcacgagtcttcttgcgttcgtcccaaccggctgggggatcttgctgatcgcacaggcgttgaggcctgcgctgcagaagatcgggcttatctggaacgcggttatctcccttgctcggttatatgacatactgttcgggatagtcatcatggttcccgtagcgttcatgtcgtggatgcctgggtttcagtcgatgcaaacgaggatcttattcaatgaagcttttagcagagggcttcgtatcatgcagattgtcactgggaagaaatcaaaaggcgatgtcgaagttgaaaaaagaaggtaaagcttcttatttacccaaacatctttttatgttctgtttgtttggaatcttaaattacaacaacactaatgcaaagcttttacaacttgtgtaggtcttgaggtatatggtaatttaaaagttgctggtttgcggcgatgtgacctgttggaggttagttttgtattcttacaagttatgcttcttgtctgaataggaactcagacacccgtgttttgtcttcttcttattaaaccaggtttttggagagctttggttgaggaagctattcgattagataaatcttttagtgggggagacatatatatatacatttgtcagtactttgttagtgtgtggaagtggggacgactctgattctgattccttatgtggttattgtctgaaacgttacagcattttgtgaagtaggcttttgtgcaagatttgatctctttctctcattgtagagctttagagcattttttagtatatgtttaatctttgattttctaatgtcatttgcattcatattcacatcttcttcgttggtcttaaaccagttttcagatgcttatccattgtccacttctctatgtatctgtttcttctgtttgttttcagtattgtcttttattctttaatcagttttatttggacc

SEQ ID NO: 3 - DNA, 10431 bp, Brassica napus
  source 1..10431: mol_type = genomic DNA
agtgagtttgttctccagcgagccggagaaggcatcaacgcaaacctggaggaacagattgaccggtatgttcattaacgctgcaagccagctttgatgttagctgcgtaaagatccctgcgtttacatcaaaacattacattgtttgctgtgactaaggtaacagagtaaacgtgtaaaatactctggtgttttggagaaaacaaacttacatggagcccttaattgcatctatggcagcttgtgctgagaggaaaccttcaacgtccacttttccttgtaggttactaatatatgcatattttatttaaatcaatccaaatatatgggtaaaaaactttatttgaggatgtactatatatatgaatgcctaagagtataatgtacaaaggtgaaacttgccttgacattgaaggagtagaggactgtttgctccatggtagtcatgtaagtgtgaagaatagagagatgaagatcctccaaaaaagcaatagtcttaataagttgtcctggtcttcttcttgaaagtttcttgatcatggcatcaaactctagcagcttcacctccacatcagccaagcacgactcgttctcagctgtttcttcccggagccctcctcctcctcctatacgactcatgtgactctaggcattgtagaagttgctcaagctctcttacaaactctattccacctatgatcgacgcttgatctctctgttatatacatgcatattaaaaaatattttaaaataatgaatacaatcacgaaatgaagatgtatatgtgttatcatgatacccattgaacgtaggatccagacaaaagagacttgaggttacgaagatgctcgttcatttgcttcctattcctagcgaccgcaatatgtctcattcgttagttttctacttcttcgcttgtcttgcttgtttttgctcttcacctcctttgtcatatcattcttgttctcactatctccttcttctcctccagtaaaccgacttgttattgtcgttaacgtctaaggattcacctaaaaaatttggtgcctgaaagaaaataaaggtgaagaaataattattaagataatataggtcaattgacttggaatcaaaattttcaccctgcaagttgacatggtttcatactaatgtgatacattaagacatagtttatggttttaatgattgttacctgatccatcaggcattataccaaaaggacaggcacggaggaactattcagttagcccatgctcctgcacaaatttatcaatcaactcatatctagaaggatatatgagaaaagaagtttctggtgttattaggagttgtgaggatgatgtttattaagaaataaactatataagcctttacatataaattacaaaataccttgattctcatccaagattccggtgtggatcgatttatcaatgacatatttgcaaaaaaaaataatagggttacctggaagatgggtgttggtgtttgctagaccaagcaactcatctttgcagaacctgaaacgttcttgaggaattggtgaacctgggtcagttaagtctttcaaacccgtctcaatattgaaccggatcgacacaggagagtcggacagcttgaagtccggacagctcaaagccgctaacagagtacacttaaccaacagtgaaaagatgtctgaatgtgtttaagcggtagacgttcacggtggcttaggtgagagtggcctgcaaagcatgaaaaattggaaacataaacaaatgaaataaatattgcaatcattaaaaataaactaaagcgcttgttaactttttcataatcaaaagacataagcatatggatgtctctacacggactatttgaattttaggtatgaataaaatatacgaaaaaagtaaaaaagaatcaacaatcacaacaatgatcatatcccaaatatttgaagataacaaaaacttatggagaataacgaagacatgaatgtattgatctccggtgaagaactattaggtttgaaagtttcataaccaaaacacaacaacgcgagaaaggattaaaaaacaaaacaaaactacatagtcaagccggatttaccttggcgtccatgaagagaatgttgaccctctctgtcgcgcttaacattcctatcttcccagaacctaagcaaacggacctgggcggtggaggtgcagcggctagctttcaaatcggcgagaaagacggaggaattagccattatcgactttgatttcttagaaaacagtttatgggaatgtagtaggattgtgagaatgatgtttctgggaagagcacatacctttttgtaggaggagcttcaccgcttgtaagagagatgagagtattgagtgagaaatcgttgagcttcatcgtggaggatggatttaaaaagggctttaagagtaagaatctgtaacaggtagattgctggatccgtaggaagaagatgaagcatctcgatcaaaaccctagggtatgatattgcaacggcggagaagagaatgagatgaaaccatagaattaaaaactctgcgtttcaggcatacgtcttactctgctttttaaagattgggctttataaaccaaaacaataacaaagaatgtagcccaaacggaaacagaataaatgaaacgcggtaaagtgaacagatgtcacataatgattggctgatttaattgtcctacgtggacagcttctctgatgctcatataacccttttagtataggttagatgatatcatccttagtcaaaagtcaaaacatctcaaattaaataagtatagagttgtgaatttttaaaaaagtatagagtatataaaattagcatttgcgaatgtttttaaaagtttggcgtttgtatggatcactgtcagtgtcatgaatcattcggtgactgcaaaattttatatgacaaaaaaaaaagagagttgatacttaccattctcgatattttgaaatttttttcgtcgttaatacaaactcaacattttcgaaatcttggattaaaaaaaaaaaatatggacaaaacggtacttattaattaattatgtactagcgtcttgacattattaatatagatataattataagaacaagaaacgtatatgcacaaaccctcaaaccaaatttaaatagtgttaaaaaacaagggatgtacttgttatgaaaaattaatatataaccattcttaacatttttacctaacccttaaactttataaataaaaatacgtaatatcccgatagaaatattttaaaattttgatttgattatttatgttatcaaagtgcatttatttttttttgtcacgtatatatttaaaatttcaacattaaaagtgtgtaagatggagtagtgaaaatatagacgacttaccataaaattatttgtgatatcatttgaataagactaaaacacggacaaaaataatatagtgattgcattgttattaaacaattatttggatttgaaaagaaataactcattgaatgaagcctatgagccgagtaaacggacgtggatagcttaagcggttggaccgtacaaaatttatgaccaacaagaaaatgaaattgattttttttttgacgaaaaaatgaaattgattttagaaagtaacaaaatggttccagatttcaatacttcaacgtttttttatgccgactttcttttcctcttttacttacatgataaggaaacgagagaggttgaagaaacaaagaaacttcctaagcatttagaaatgtatcttcaaaaaatctagtatttaccaagtaaaatcattcaaaataaggaaaaaaagaagaatctatttcccaatttggttttcaccgaaaaaaaaattgtcttaataaaatcttaatgcaccaaatcatataagaactttatatgaccattgtcattagttttttctttatggtaatggatcttcaaataatttaaattaaattaaaaaaaaaaaagagaaacaaaaacgcggaaaggagagcccaaatcgtctccgtctgtcattgtcgcagtctctcacagctaaataaaagagaaaaacaaatcgcaacgcttcaatatctctctcccaatcgtttgaatcagtcttcctcatcacaatcccccaaagatccaccacacatgctactatgagcctccgccaccgcaccgtcccctctcaacccggacggcccccggcggcgggcgcaatcgacgacgagccctacaacatcatccccgtcaacaacctcctcgccgaccacccctccctccgctaccccgaggtccgcgccgccgccgccgccctcaaaaccgtcggagacctccgccgccccacctacgtccaatggcgcccccactacgacctcctcgactggctcgccctcttcttcggcttccagaaggacaacgtccgcaaccagcgcgagcacctcgtcctccacctcgccaacgcccagatgcgcctcacgccgccgccggataacatcgattccctcgatcccgccgtcgtccgccgtttccgccgcaagctcctcggtaactactcgagctggtgctcgtacctcgggaggaagtcgaacatctggatctcggatcggaaccccgattcgaggcgggagcttctctacgtcggcctctacctcctcgtgtggggggaggcggcgaatcttaggtttatgccggagtgtgtctgttacatcttccacaatatggcctcggagcttaacaagatcctcgaggattgcctcgacgagagcacggggcagccgtactctcctagaatcacgggggagaatagtttcctaaacggcgtcgttaaacctatttacgagacgatcaaagctgagattaacgagagcaagaacgggacggagccgcattgtaagtggaggaactatgatgatattaatgaatacttttggacggataggtgttttagtaaattgaaatggccgattgatttggggagcagtttcttcaagagtagtagagggagaggcgttgggaagacaggttttgtggagaggaggacgttcttttacctctacaggagctttgataggctttgggtgatgcttgctttgtttcttcaagctgctattatagtcgcttgggaggagaagccgggtggagggtcggtgaggagtcagctctggaatgcgttgaagtcgagggatgttcgggtgaggcttttgactgtgttcttgacgtggagtgggatgagattactgcaggctgtgctggacgctgcctcgcaacggccgcttatttctagagagaccaagcggctgtttttcagaatgttgatgaaggttgtagctgctacggtttggataattgcttttattgttctctacacgaacatctggaagcagaggaagcaagacaggcagtggtccagagccgcgaatgacaagatctatcagttcctttacgctgtggtggctttcttggtccctgagatcctggctttggctctgtttatagtcccgtggataaggaactttctggaagagaccaattggaagatattctttgctttgacttggtggttccagggtaaaagctttgtgggtcgaggtttgagagaggggttggtggacaacatcaagtactcgactttctggatctttgtcctagcaacgaagttcacgttcagctacttccttcaggttaagccaatgattaaaccctcgaagctgctatggaatttgaaggaggtggattatgagtggcatcagttctttggcaagagcaataggttttctgtcttgttattgtggctgccagtggtgttgatatacctgatggatatccaaatttggtacgcgatctattcttcgattgttggtgctgttgttgggctgtttgatcatctgggggagatcagggacatgggacagcttaggctgaggtttcagttctttgctagcgctattcagttcaacctaatgcctgaggaacaactcctgaatgctagaggatttggtaacaagcttaaggacgccattcataggtaagtctattgaagcatgttactgatattcctttataatttactgtacagagtttgtctttacggtacaagttatggaattttagttttgttaaagcagattctcttaactcggcttttcaacattgtaataaccttgagtttgtgtactttatgcagattgaagctgaggtatggacttgggcggccatttaagaaactcgagtctaatcaggttgaggctaacaagtttgcgctgatctggaatgagataatcttagctttcagagaagaggatatagtctctgatcgagaagtagagctactggagctgccaaaaaattcctggaatgtgacagttatccgctggccgtgtttcctgttgtgcaacgagcttttgcttgcactgagccaggcgaaagagctggttgacgcacctgataaatggctgtggcacaagatatgcaagaacgagtacaggcggtgtgctgtggttgaggcatatgaaagcatcaaacatctgttgctctcaatcatcaaaattgacaccgaagaacataaaattattacaattttctttcagatgattgaggtgtctattcagggtgagcagttcaccaagaccttcaaagtggacctattgccaaagatttatgagacgctacagaagttggttgggctgttgaatgatgagaaagtggatgttgggcgagtggtgaatggtctgcagtctatttatgagattgcaacacgacagttcttcatagaaaagaagacgactgaacagctatctaccgaggggttaactcctcatgatccagcctcaaagttactgtttcagaatgctgttaggcttcccgatgcaagcaatgaagacttcttccggcaggttaggcggttacacacaattctcacttctagggactctatgcacagcgtccctgtgaatctagaggcgagacggcggattgccttcttcagcaattcgctcttcatgaacttgcctcatgcacctcaggtggagaaaatgttggcgttcagtgttatgactccatactacagcgaggaagttgtatacagtaaagaacagctccgaaatgagactgaggatgggatttcaaccttgtattacctgcagacgatttatgccgacgaatggaaaaattttaaggaacggatgcgtagggaaggtataaagacagatgttgagttgtggacaaccaagctgagagagctcaggctttgggcttcctacagaggtcagactttggcacgtacagttcgaggaatgatgtactattacagggctcttaagatgcttgcttttctcgactctgcgtctgaaatggacattcgggaggatgctcaggagcttggttcaatgaggagttcgcagggaaatcgattggatggtgttgacgatgtaaatgaccgatcttctctaagcagagcaactagctctgtgagcatgctgtataaaggccatgagcatgggactgcattgatgaaattcacatatgtcgtggcgtgccaaatctatgggtctcaaaaagcgaagaaagagcctcaggcagaggaaattctgtatcttatgaagcaaaacgaagcccttcgtattgcatatgtggatgaggtacatgcgggcaggggagagactgagtattactccgttctggtgaaatacgatcacacgttggagagggaagtggagatattccgtgtgaagctacctggtccggtgaagctgggtgagggaaagccagagaaccagaatcatgcaatgatctttacccgtggtgatgctgttcagaccatagatatgaaccaggataattattttgaggaggctctcaagatgagaaatttgctccaggagtttaggcattatcatgggatcagaaaaccaactattcttggtgtcagagagcacatcttcacgggttctgtctcgtctctggcttggttcatgtctgctcaggagacaagtttcgtcactcttggtcagcgtgttctagccaacccgctgaaggtcagaatgcattatggtcaccctgatgtatttgacagattctggttcttgagtcgaggtggcatcagcaaagcttctagagttataaatatcagtgaggacatcttcgccgggtttaattgcacattgcggggcggtaacgtcacccaccacgagtatattcaggtagggaaatgttcatcgtttggatattctaactaatttatacatcgacaacaatactataattccacttttttgttataacctttttgtgtgtgcatatgtattcaggttgggaagggtcgagatgttggattgaatcaaatatcaatgtttgaggctaaggtagccagtgggaatggagagcaggttcttagccgagatgtgtacaggttgggtcataggctcgatttcttcagaatgttatcatttttctacacaacggtggggtttttcttcaacacgatgatggtcattctcactgtctacgctttcctctggggccgggtttatcttgctctgagcggtgttgagaagtccgctctagcagacagcacagacaccaacgcagcgcttgctgtgatattgaaccagcagttcatcattcagcttggtctcttcacagctctgccaatgattgtggaatggtctctcgaggagggtttccttctcgcgatatggaacttcattcggatgcagattcagctttcttctgtcttctacacattctcaatggggaccagagctcactattttggccgaaccattctccacggtggagcaaagtacagagccactggacgtggatttgttgtcgagcacaagagtttcactgaaaactaccgtctatacgcgcgcagtcactttgtgaaggccatcgagcttgggctgatcctcatagtctacgctacgcacagtcccatcgccaaagactcattgatctatatagccatgactctcaccagctggttcctcgtgatttcatggatactagccccttttgtgttcaacccgtcaggtttcgactggcttaagacggtctacgacttcgaaggcttcatgaactggatctggtatcaaggcagaatctcaacgaagtccgaacagagctgggagatatggtggtatgaggaacaggaccacctgagaaccaccggtctaccaggaagaatcatggagataatcttggaccttcggtttttcttcttccagtacgggattgtataccagctcaaaatcgcaaacggatcaaccagcattctcgtctacttactctcatggatatacatcttcgcagtgtttgtgttcttcctggtaatccaatacgcccgtgacaagtactcagcgagaaaccacatacggtacaggctcgttcaattcctcctgatcgtgtttggtacactggtgattgttgctctcctggagttcacgcatttcagcttcgtggatatcttcacgagtcttcttgcgttcgtcccaaccggctggggaatcttgctgatcgcacaggctttgaggcctgcgctgcagaagatcgggcttatctggaacgcggttatctcccttgctcggttatatgacatactgttcgggatagtcatcatggtccccgtagcgttcatgtcgtggatgcctgggtttcagtcgatgcaaacgaggatcttattcaatgaagcttttagcagagggcttcgtatcatgcagattgtcactgggaagaaatctaaaggcgatgtcgaagttgaaaaaagaaggtaaagcttttataacttgtgttctgtttatgtttgtttggaatcttaaattacaacaacaccaatgcaaagcttttataacttgtttaggtcttaaggtatatggtaacttaaaagtcgttggttttgcggcgatgtgatcagttggaggttagttttctcaagacatggaagttttataagttattgcttcttgtgtgaagaagaagtcatatacatccatgttttgttttgtcttcttcttaaagcaggtttttggagagctttggttgaggaatccggaagttagataatcttttagtgggggagacatatatatacacatttgtcagtactttgttagagtgtgtggaagtggggacgactctgattctggttccttatgtggttattgtctgaaacgttacagcattttgtgaagtaggcttttgtgcaagatttgatctctttctctcattgtagagctttagagcattttttagtatatgtttaatcttttgattttctaatgtcatttgcattcatatttcacatcttcttcgttggtcttaaaccagttttcagatgcttatccattgtccacttttctatgtatctgtttcttctgtttgttttcagtattgtcttttattctttaatcagttttatttggaccacaacacca

SEQ ID NO: 4 - DNA, 9344 bp, Brassica rapa
  source 1..9344: mol_type = genomic DNA
aaagtaataaaatggttccaaatttcaatacatcaatggttttctaatgctgacttgcttttcctcttttacttaaacgataaggaaacgagagaggttgaagaaacaaagaaacttcctaagcatttacaaatatatcttcaaaaaatctagtatttaccaagtaaaatcatacaaaataagaaaaaaaaagaagaatctatttcccaatttggttttcaccggaaaaaaaaaactgtcttaacaaaatcttaatgcaccaaatcatataagaactttatttgaccattgtcattagtttttttccttatggtaatggatcttcaaataatttaaattaaattaaaaaaaaagaaaaaagaaacaaaaacgcggaaaggagagcccaaatcgtctccgtctgtcattgtcgcagtctctaaaagagaaaaacaaatcgcaacgcttcaatatctctctctctctccctatcgtttgaatcagtctcccaagatccaccaccacacatgctactatgagcctccgccaccgcaccgtcccctcacaacccggacggcccccggcggcgggcgcaatcgacgaggagccctacaacatcatccccgtcaacaacctcctcgccgaccacccctccctccgctaccccgaggtccgcgccgccgccgccgccctcaaaaccgtcggcgacctccgccgccccacctacgtccaatggcgcccccactacgacctcctcgactggctcgccctcttcttcggcttccagaaggacaacgtccgcaaccagcgcgagcacctcgtcctccacctcgccaacgcccagatgcgcctctccccgccgccggacaacatcgattccctcgatcccgccgtcgtccgccgcttccgccgcaagctcctcggtaactactccagctggtgctcctacctcgggaggaagtccaacatctggatctcggatcggacccccgattcgcggcgggagcttctctacgtcggcctctacctcctcgtgtggggcgaggcggcgaatcttaggtttatgcctgagtgtatctgttacatcttccacaatatggcctcggagcttaacaagatcttagaggattgcctcgacgagagcacggggcagccgtattctcctaagatcacgggggagaatagtttcctaaacggcgtcgttaagtcagaacaaaaggaagattccatctaatggattataatcccacatgcccatttttaaaactagacaaaataacttataatagtcccataagagcattaagacaaactgaaccaaaaagaatcttaaatgctggtgaatctagtctcgaatgctgctgaacactcctgatattagaatttggcagaaaatattcatactaataacagtaaatacatttcaaaacaagaaaatagtagtagtaaataaaaacaattaatatgttattattgtttctcttagctgttaccattacattaacaatttacattcaatccttttaatgaaaaatattataaaatattaaaagaagttattagaaaaggagagggatatggggggggggagagcatggaggggtcccatgtgtgtttcgaaaagcagcaaacatgactttgtaaagtcttgttttagctagagttccaaaaaaaaaagagttcaaaaaaaaagaaaaaaaaaagtcttgttttagcgtccaaagcggtctcactcgccgttcagctgttaaattttcttgaaataaatcgctccacttgttttttatttttgtttctttcaacccacttgtattaataacaataatgatattgctaatcttgaacaaatcgtggattgtcttttcttccagtgatatcatccttagtcaaaacatctcaaattaaataaatatatagttgtgaattaaaaaaaaaggtatagagtttataaaattagcatttgcgaatgtttttaaaagtttggcgtttgtatagatcactgtcagtgtcatgaatcattcggtgacagcaaatttgtatatgccaaaaaagagagttgatacttgccattctcgatattttgaattttttttcgtcgacatacaaactcaacattttcgaaatcttagattaaaaacaaaatggacaaaacgatacttattaatgaattatgtactagcgtctaaacattagtaacatagatataattataagaacaagaaacatatatgcacaaagcctcaaaccaaatttaaatagtgttacgaaacaagggatgtacttgttatgaaaaattaatgtataaccattcttagcattttttacctaacccttaaactttataaagaaaaatatgtaacatttcggtgtgtggtatataaaagtattttgaaaatcgatttgattatttctattattaaaatgtacttactttttttatcacacatttatttacaatttcagagttaaacatgattaagacaaagtagtgaaaatatagatgatctaccagaaaatcatttgctttctgagtgagatcaaaataaaggcaaaaataatgtgatgattgtatattaaacaattattttgaatttgaaaagaataactcatcgaatgaagcctatgaaccgagtaagcggtgaagcggttggaccgtcacaaaaattatgaccaacaagaaaataaaattgatattagtgaatgatatattttggaaagtaataaattggttccagatttcaatacatcaatgtttttctaatgctgacttgcttttcctctttttcttacatgataaggaaacgagagaggtcgaagaaacaaagaaacttcctaagcatttagcaacgcatcttcaaaaaatctagtatttaccaagtaaaatcatacaaaataaggaaaaaaagaagaatatatttcccaatttggttttcaccggaaaaaaaaaactgtcttaacaaaatcttaatgcaccaaatcatataagaactttatttgaccattgtcattagttttttccttatggtaatggatcttcaaacaatttaaattaaattaaaaaaaaaaagaaaagagaaacaaaaacgcggaaaggagagcccaaatcgtctccgtctgtcattgtcgcagtctctaaaagagaaaaacaaatcgcaacgcttcaatatctctctctctctctctccctatcgtttgaatcagtctctcaagatccaccaccacacatgctactatgagcctccgccaccgcaccgtcccctctcaacccggacggcccccggcggcgggcgcaatcgaggacgagccctacaacatcatccccgtcaacaacctcctcgccgaccacccctccctccgctaccccgaggtccgcgccgccgccgccgccctcaaaaccgtcggcgacctccgccgccccacctacgtccaatggcgcccccactacgacctcctcgactggctcgccctcttcttcggcttccagaaggacaacgtccgcaaccagcgcgagcacctcgtcctccacctcgccaacgcccagatgcgcctctccccgccgccggacaacatcgattccctcgatcccgccgtcgtccgccgcttccgccgcaagctcctcggtaactactccagctggtgctcctacctcgggaggaagtccaacatctggatctcggatcggacccccgattcgcggcgggagcttctctacgtcggcctctacctcctcgtgtggggcgaggcggcgaatcttaggtttatgcctgagtgtatctgttacatcttccacaatatggcctcggagcttaacaagatcttagaggattgcctcgacgagagcacggggcagccgtattctcctaagatcacgggggagaatagtttcctaaacggcgtcgttaagcctatctacgacacgatcagagctgagattaatgagagcaagaacgggacggagccgcattgtaagtggaggaactacgatgatattaatgagtacttctggacggataggtgttttagtaaattgaaatggccgattgatttggggagcagtttcttcaagaacagcagaggtagcggagttgggaagacaggttttgtggagaggaggacgtttttctacctctacaggagctttgataggctttgggtgatgcttgctttgtttcttcaagctgctattatagttgcttgggaggagaagccgggtggagggtcggtgacgagtcagctctggaatgcgttgaagtcgacggatgttcaggtgaggcttttgactgttttcttgacgtggagtgggatgaggttgttgcaggctgtgctggacgctggctcgcaacggtcgcttatttctagagagaccaaacggctgtttttcagaatgttgatgaaggttgtggctgctacggtttggataatagcgtttattgttctctacacgaacatctggaagcagaggaagcaagataggcagtggtccagagccgcgaatgataagatctatcagttcctttacgctgtggtggctttcttggtccctgagatcctggctttggctctgtttatagtcccgtggataaggaactttctggaagagaccaattggaagatattctttgctttgacttggtggttccaggggaaaagctttgtgggtcgaggtttgagagaggggttggtggacaacatcaagtactcgactttctggatctttgtcctcgcaacgaagttcacgttcagctacttccttcaggttaagccaatgattaaaccctcgaagctgctatggaatttgaaggaggtggattatgagtggcatcagttctttggcgagagcaataggttttctgtcttgttattgtggctgccagtggtgttgatatacctgatggatatccaaatttggtacgcgatctattcttcgattgtcggtgctgttgttgggctgtttgatcatctgggggagatcagggacatgggacagcttaggctgaggttccagttctttgctagcgctattcagttcaacctaatgcctgaggaacaactcctgaatgctagaggatttggtaacaagcttaaggacgccattcataggtaagtctattgaagcatgttactgatattcctcctatataatttactatacagagtttgtctttacagtacaagctataggattttagttttgttaaagcagattctccgcaatgaactagactttttcacattgtaataacattgagtttgtgtactttatgcagattgaagctgaggtatggattggggcggccgtttaagaaactcgagtccaatcaggttgaggctaacaagtttgcgctgatctggaatgagataatcttagctttcagagaggaggatatagtctctgatcgagaagtagagctgctggagctgccaaagaattcctggaatgtaacagttatccgctggccgtgtttcctgttgtgcaatgagcttttgcttgcactgagccaggcgaaagagctggttgacgcacctgataaatggctgtggcacaagatatgcaagaatgagtacaggcggtgtgctgtggttgaggcatatgaaagcatcaaacatctgttgctttcaatcatcaaaattgacactgaagaacataaaattgttacaattttctttcagatgattgaggtctctattcagggtgagcagttcaccaagaccttcaaagtggaccttttgccaaagatttatgagacactacagaagttggttggtctgttgaatgatgagaaagtggatgttgggcgagtggtgaatggtctgcaatctatttatgagattgcaacacgacagttcttcatagaaaagaagacgactgaacagctatctactgaggggttaactcctcatgatccagcctcaaagttactgtttcagaatgctgttaggcttcccgatgcaagcaatgaagacttcttccggcaggttaggcggttacacacaattctcacttctagggactctatgcacagcgtccctgtgaatctagaggcgagacggcggattgccttcttcagcaattcgctcttcatgaacttgcctcatgcacctcaggtggagaaaatgttggcgttcagtgttatgactccatactacagcgaggaagttgtatacagcaaagaacagctccgaaatgagactgaggatggaatttcaaccttgtattacctgcagacgatttatgccgacgaatggaaaaattttaaggaacggatgcgtagggaaggtataaagacagatgttgagttgtggacaaccaagctgagagagctcaggctttgggcttcctacagaggtcagactttggcacgtacagttcgaggaatgatgtactattacagggctcttaagatgcttgcttttcttgactctgcgtctgaaatggacattcgggaggatgctcaggagcttggttcaatgaggagttcgcagggaaatcgattggatggggttgacgatgtaaatgacggatcttctctaagcagagcaactagctctgtgagcatgctgtataaaggccatgagcatgggactgcattgatgaaattcacatatgtcgtggcgtgccagatctatgggtctcaaaaagcgaagaaggagcctcaggcagaggaaattctgtatcttatgaagcaaaacgaagccctccgtattgcatatgtggatgaggtacatgcgggcaggggagagactgagtattactccgttctggtgaaatacgatcatacgttggagagggaagtggagatattccgtgtgaagctacctggtccggtgaagctgggtgagggaaagccagagaaccagaatcatgcaatgatctttacccgtggtgatgctgttcagaccatagatatgaaccaggataattattttgaggaggctctcaagatgagaaatttgctccaggagtttaggcattatcatgggatcagaaaaccaactattcttggtgtccgggagcacatcttcacgggatctgtctcgtctctggcatggttcatgtctgctcaggagactagtttcgtcactcttggtcagcgtgttctcgccaatccgctgaaggtcagaatgcattatggtcatcctgatgtttttgacagattctggttcttgagtcgaggtggcatcagcaaagcttctagagttataaatatcagtgaggacatcttcgccgggtttaattgcacattgcggggcggtaacgtcacccaccacgagtatattcaggtagggaaatgttcatcatttggatattctaactaatttatacatcgacaacaatactataattccactttttttgttataaccttcgtgtgtgtgcatatgtattcaggttgggaagggtcgagatgttggattgaatcaaatatcaatgtttgaggctaaggtagccagtgggaatggagagcaggttcttagccgagatgtgtacaggctgggtcataggctcgatttcttcagaatgttatcatttttctacacaacggtggggtttttcttcaacacgatgatggtcattctcactgtctacgctttcctctggggtcgggtttatcttgctctgagcggtgttgagaagtccgctctagcagacagcacagacaccaacgcagcgcttgctgtgatattgaaccagcaattcatcattcagcttggtctcttcacagctctgccaatgattgtggaatggtctctcgaggagggtttccttctagcgatatggaatttcattcggatgcagattcagctttcatctgtcttctacacattctcaatggggaccagagctcactattttggccgaaccattctccatggtggagcaaagtacagagccactgggcgtggatttgttgtcgagcacaagagcttcactgagaattaccgtctatacgcacgcagtcactttgtgaaggccatcgagcttgggctgatcctcatagtctacgctacgcacagtcccatcgccaaagactcattgatctatatagctatgactctcaccagctggttcctcgtgatttcatggatactggccccttttgtgttcaacccgtcaggattcgactggcttaagacggtctatgacttcgaaggcttcatgaactggatctggtatcaaggcagaatctcaacgaagtccgaacagagctgggagatatggtggtatgaggaacaggaccacctgagaaccaccggtataccaggaagaatcgtggagataatcttggaccttcggtttttcttcttccagtacgggattgtataccagctcaaaatcgcaaacggatcaaccagcattctcgtctacttactctcatggatatacatcttcgcagtgtttgtgttcttcctagtaatccaatacgcccgtgacaagtactcagcgagaaaccacatacggtacaggctcgtccagttcctcctgatcgtgtttggtacactggtgattgttgctctcctcgagttcacgcatttcagcttcgtggatatcttcacgagtcttcttgcgttcgtcccaaccggctggggaatcttgctgatcgcacaggctttgaggcctgcgctgcagaagatcgggcttatctggaacgcggttgtctcccttgctcggttgtatgacatactgttcgggatagtcatcatggttcccgtagcgttcatgtcgtggatgcctgggtttcaatcgatgcaaacgaggatcttattcaatgaagcttttagcagagggcttcgtatcatgcagattgtcactgggaagaaatcaaaaggcgatgtcgaagttgaaaaaagaaggtaaagcttcttatttacccaaacatcttttatgttctgtttgtttggaatcttaaattacaacaacactaatgcaaagcttttataacttgtgtaggtcttgaggtatatggtaatttaaaagttgctggtttgcggcgatgaggttagttttgtattcttataagttatgcttcttgtctgaataagaactcagacacccgtgttttgtcttcttcttattttaaaccaggtttttggagagctttggttgaggaagctatttgattagttcatcggttagtgggagaaacatatatatacatttgtcagtactttgttagagtgtgtggaaatggggacgactctgattctgattccttatgtggttattgtctgaaacgttacagcattttgtgaagtaggcttttgtgcaagatttgatatctttctctcattgtagagctttagagcattttttagtatatgtttaatctttgatttttctaatgtcatttgcattcatatttcac

SEQ ID NO: 5 - DNA, 8624 bp, Brassica oleracea
  source 1..8624: mol_type = genomic DNA
gtttctttttttttttggtcgaaattatattattgtttctctgagctgttaccattacattaacaatttacattctatccttttgatgaaaaatattataaaatgttaaaagaagttattagaaaaggagaggaaaacagggggagagcatggaggggtcccatgtgtgtttcggaaagcagcaaacatgactttgtaaagtcttgttttagcgtccaaagcggtctcactcgccgttcagcgagctttagctgttaaattttcttgaaataaatcgctccacttgttaaaaaaaaaaatgataactgtgtccatttcgactaatatctattttgtgtcatttttgcatataatattttttaatatttttgaaaataaatttaataaatagttttataaacaaaaaaaattgaaaaatagtaactattgataaaatacctatatgaacttagtagtattttttttcagttctaaaaatgtttaaatcataattttcagattatgttataaataaagcagaaatgacattctacgaagtagaaaacgaaatccattttttttcattgaatctacaatgtttagaatacgaaatcttcgtaaataagagtattctaaaaatatttagaatacacattccgcgcttaacctaccgatctaaaatctttagaaatcaaaatctacagattaatataaatctaaaacacgttgaaaccgacttctacagatttactgtaaatctaaaacatgtagaaatcaaaattctacacattactataattctaaaaacctatagaaaccgatttctacatattatctgtattctacggataagaaatcagttcttaaaaatatggaaacaaaatatttgagaatattcactttcatattttgaaaaaaatcgatttaaaaaaaacaaaaaaaacaaaaaaccttcttttcacgccgtcttctatattccattgattgttcacaaaattttcacattatttccaccatatcagtgtgaaattgagtgagttagggtttatgaacttgagactttgattttctccctttttgttcgtgaaggagatgagaaattatgagtttcatctcaaagaaatcgagtttaaagagttgaaatcgtgtttggtcggttcaaatcaagtgggaattaaaccggatataaccgaagaaaaccaggaaatcgatttggaatacttacctaggcacaagatcgatcggtctataattagagatcggttgatctgtatgaaatcgacttttgccgatcgagtgaaatggaccaggtcgatcagccgcgtttttttgttctgcataatgatcagaatcggtcaatccgtccgaccttgtaatttcggatcgatcgatcccgcttgatcgaacctattatttattatatttaagaattacaattttaaggattttattgccattttgaaaataattagtctaattgaacataaaatatataagattagtctaaaatgacatagttatcattttttgttctaaaaaaacaatttcccttttttatttttgtttctttcaacacacttgtattaataacaataatgatattgctaatcttgaacaaatcgtggattgtcttttcttccggtgatatcatccttagtcaaaacatctcaaattaaataagtatagagttgtgaatgtttttaaaagtttggcgtttgtatagatcactgtcagtgtcatgaatcattaggtgactgcaaatttttatatgacaaaaaagagagttgatacttgccattctcgagattttgaagtttttttcgtcgtcaacatacaaactcaacattttcgaaatcttggattaaaaataaaaatatggacaaaacgatacttattaattaattatgtactagcgtcttaacattattaatatagatataattataagaacaagaaacataaacatatatgcacaaagcctcaaagcaaatttaaataatgttacaaaacaagggatgtacttgttatgaaaaattaatatataaccattcttaacatatttttacgtaaaccttaaactttataaagaaaaatatgtaacattccggtgcgtggtatatagaaatatttttaaaaattgatttgactatttccattagcaaaatgcatttattttttcgatcatacatttatttaaaatttcagggttaaacgtgattaaaatggagtaataagaatatggatgatctaccagaaaatcatttacgttttaagtgagaccaaaatacggataaaaatcatgtgatgattgtatggttattaaacaattattttgaatttgaaaataattaactcatcaaattaaacctaaggaccgagtaagcggacgcagatagccgtaagcggttgggccgtcacaaaatttatgaccaacaagaaaatgaaactgattttagtggatgatatattttagaaagtaataaaatggttccagatttcaatacttcaatggtttttttatgctgactttcttttcctcttttacttacatgataaggaaacgagagaggttgaagaaacaaagaaaattcctaaggatttagaaatgtatcttcaaaaaatctaagtatttaccaagtaaaatcatacaaaataaggaaaaaaaaagaatttatttcccaatttggtttttaccggaaaaaaaattgttttaataaaatcttatgcaccaaatcatataagaacgaaccataacaaactttatttgaccattgtcattagtttattttatttatggtaatggatcttcaaataatttgaattaaattaaaaaaaagaataaaaaaagagaaacaaaaacgcggaaaggagagcccaaatcgtctccgtctgtcattgtcgcagtctctcacagctaaataaaagagaaaaacaaatcgcaacgcttcaatatctctctcccaatcgtttgaattagtcttcctcatcacaatacccctaagatccacctcacatgctactatgagcctccgccaccgcaccaccgtcccctctcaacccggacggcccccggcggcgggcgcaatcgacgaggagccctacaacatcatccccgtcaacaacctcctcgccgaccacccctccctccgctaccccgaggtccgcgccgccgccgccgccctcaaaaccgtcggagacctccgccgccccacctacgtccaatggcgcccccactacgacctcctcgactggctcgccctcttcttcggcttccagaaggacaacgtccgcaaccagcgcgagcacctcgtcctacacctcgccaacgcccagatgcgcctcacgccgccgccggataacatcgattccctcgatcccgccgtcgtccgccgtttccgccgcaagctcctcggtaactactcgagctggtgctcgtacctcgggaggaagtcgaacatctggatctcggatcggaaccccgattcgaggcgggagcttctctacgtcggcctctacctcctcgtgtggggggaggcggcgaatcttaggtttatgccggagtgtatctgttacatcttccacaatatggcctcggagcttaacaagatcctcgaggattgcctcgacgagagcacggggcagccgtattctcctagaatcacgggggagaatagtttcctaaacggcgtcgttaaacctatttacgagacgatcaaagctgagattaacgagagcaagaacgggacggagccgcattgtaagtggaggaactatgatgatattaacgagtacttttggacggataggtgtttcagtaaattgaaatggccgattgatttggggagcagtttcttcaagagtagtagagggagcggcgttgggaagacaggttttgtggagcggaggacgtttttttacctctacaggagctttgataggctttgggtgatgcttgctttgtttcttcaagctgctattatagttgcttgggaggagaagccgggtggagggtcggtgaggagtcagctctggaatgcgttgaagtcgacggatgttcaggtgaggcttttgactgtgtttttgacgtggagtgggatgagattactgcaggctgtgctggacgctggctcgcaacggccgcttatttctagagagaccaaacggctgtttttcagaatgctgatgaaggttgtagctgctacggtttggatcattgcttttattgttctctacacgaacatctggaagcagaggaagcaagacaggcagtggtccagagccgcgaatgacaagatctaccagttcctttacgctgtggtggctttcttggtccctgagatcctggctttggctctgtttatagtcccgtggataaggaactttctggaagagaccaattggaagatattctttgctttgacttggtggttccagggtaaaagctttgtgggtcgaggtttgagagaggggttggtggacaacatcaagtactcgactttctggatctttgtcctagcaacgaagttcacgttcagctacttcctgcaggttaagccaatgattaaaccctcgaagctgctatggaatttgaaggaggtggattatgagtggcatcagttctttggcaagagcaataggttttctgtcttgttattgtggctgccagtggtgttgatatacctgatggatatccaaatttggtacgcgatctattcttcgattgttggtgctgttgttgggctgtttgatcatctgggggagatcagggacatgggacagcttaggctgaggtttcagttctttgctagcgctattcagttcaacctaatgcctgaggaacaactcctgaatgctagaggatttggtaacaagcttaaggacgccattcataggtaagtctattgaagcatgttactgatatttctatataatttattatacagagtttgtctttacagtacaagctataggattttagttttgttaaagaagatttctccgcaatgaactcgacttttcacattgtaataacattgagtttgtgtactttatgcagattgaagctgaggtatggattggggcggccgtttaagaaactcgagtccaatcaggttgaggctaacaagtttgcgctgatctggaatgagataatcttagctttcagagaggaggatatagtctctgatcgagaagtagagctgctggagctgccaaagaattcctggaatgtgacagttatccgctggccgtgtttcctgttgtgcaacgagcttttgcttgcactgagccaggcgaaagagctggttgacgcacctgataaatggctgtggcacaagatatgcaagaatgagtacaggcggtgtgctgtggttgaggcatatgaaagcatcaaacatctgttgctctcaatcatcaaaattgacaccgaagaacataaaattgttacaattttctttcagatgattgaggtgtctattcagggtgagcagttcaccaagaccttcaaagtggacctattgccaaagatttatgagacgctacagaagttggttgggctgttgaatgatgagaaagtggatgttgggcgagtggtgaatggtctgcagtctatttatgagattgcaacacgacagttcttcatagaaaagaagacgactgaacagctatctaccgaggggttaactcctcatgatccagcctcaaagttactgtttcagaatgctgttaggcttcccgatgcaagcaatgaagacttcttccggcaggttaggcggttacacacaattctcacttctagggactctatgcacagcgtccctgtgaatctagaggcgagacggcggattgccttcttcagcaattcgctcttcatgaacttgcctcatgcacctcaggtggagaaaatgttggcgttcagtgttatgactccatactacagcgaggaagttgtatacagtaaagaacagctccgaaatgagactgaggatgggatttcaaccttgtattacctgcagacgatttatgccgacgaatggaaaaattttaaggaacggatgcgtagggaaggtataaagacagatgttgagttgtggacaaccaagctgagagagctcaggctttgggcttcctacagaggtcagactttggcacgtacagttcgaggaatgatgtactattacagggctcttaagatgcttgcttttctcgactctgcgtctgaaatggacattcgggaggatgctcaggagcttggttcaatgaggagttcgcagggaaatcgattggatggtgttgacgatgtaaatgaccgatcttctctaagcagagcaactagctctgtgagcatgctgtataaaggccatgagtatgggactgcattgatgaaattcacatatgtcgtggcgtgccaaatctatgggtctcaaaaagcgaagaaagagcctcaggcagaggaaattctgtatcttatgaagcaaaacgaagcccttcgtattgcatatgtggatgaggtacatgcgggcaggggagagactgagtattactccgttctggtgaaatacgatcacacgttggagagggaagtggagatattccgtgtgaagctacctggtccggtgaagctgggtgagggaaagccagagaaccagaatcatgcaatgatctttacccgtggtgatgctgttcagaccatagatatgaaccaggataattattttgaggaggctctcaagatgagaaatttgctccaggagtttaggcattatcatgggatcagaaaaccaactattcttggtgtcagagagcacatcttcacgggttctgtctcgtctctggcttggttcatgtctgctcaggagacaagtttcgtcactcttggtcagcgtgttctagccaacccgctgaaggtcagaatgcattatggtcaccctgatgtatttgacagattctggttcttgagtcgaggtggcatcagcaaagcttctagagttataaatatcagtgaggacatcttcgccgggtttaattgcacattgcggggcggtaacgtcacccaccacgagtatattcaggtagggaaatgttcatcatttggatattctaactaatttatacatcgacaacaatactataattccacttttttgttataacctttttgtgtgtgcatatgtattcaggttgggaagggtcgagatgttggattgaatcaaatatcaatgtttgaggctaaggtagccagtgggaatggagagcaggttcttagccgagatgtgtacaggttgggtcataggctcgatttcttcagaatgttatcatttttctacacaacggtggggtttttcttcaacacgatgatggtcattctcactgtctacgctttcctctggggccgggtttatcttgctctgagcggtgttgagaagtccgctctagcagacagcacagacaccaacgcagcgcttgctgtgatattgaaccagcagttcatcattcagcttggtctcttcacagctctgccaatgattgtggaatggtctctcgaggagggtttccttctcgcgatatggaacttcattcggatgcagattcagctttcttctgtcttctacacattctcaatggggaccagagctcactattttggccgaaccattctccacggtggagcaaagtacagagccactggacgtggatttgttgtcgagcacaagagtttcactgaaaactaccgtctatacgcgcgcagtcactttgtgaaggccatcgagcttgggctgatcctcatagtctacgctacgcacagtcccatcgccaaagactcattgatctatatagccatgactctcaccagctggttcctcgtgatttcatggatactagccccttttgtgttcaacccgtcaggtttcgactggcttaagacggtctacgacttcgaaggcttcatgaactggatctggtatcaaggcagaatctcaacgaagtccgaacagagctgggagatatggtggtatgaggaacaggaccacctgagaaccaccggtctaccaggaagaatcatggagataatcttggaccttcggtttttcttcttccagtacgggattgtataccagctcaaaatcgcaaacggatcaaccagcgttctcgtctacttactctcatggatatacatcttcgcagtgtttgtgttcttcctggtaatccaatacgcccgtgacaagtactcagcgagaaaccacatacggtacaggctcgttcagttcctcctgatcgtgtttggtacactggtgattgttgctctcctggagttcacgcatttcagcttcgtggatatcttcacgagtcttcttgcgttcgtcccaaccggctggggaatcttgctgatcgcacaggctttgaggcctgcgctgcagaagatcgggcttatctggaacgcggttatctcccttgctcggttatatgacatactgttcgggatagtcatcatggtccccgtagcgttcatgtcgtggatgcctgggtttcagtcgatgcaaacgaggatcttattcaatgaagcttttagcagagggcttcgtatcatgcagattgtcactgggaagaaatcaaaaggcgatgtcgaagttgaaaaaagaaggtaa

SEQ ID NO: 6 - DNA, 9270 bp, Brassica juncea
  source 1..9270: mol_type = genomic DNA
ctgtcagatttagtttcacaaaatctttgtaacttcgcggtttttgatatggaagttgctacaaaaactgacttgtgatattataatctttcggcaagaaaaatatataatactaatttcagaaagtattctgttgaattgttgatcaccaaatacacataaaccggcagatgttgacatgtttacgtgggatcactgggatagaagttttgcttctgtattagtatcgcgaaccggtttgtagtagcgaaaataacattattattttcagatttcatcatcgtcaaattcaacgttacatttgaacaggcttgtaaaaagtggacatgtggcatgagtaaaagataaagaaaattgtgagcctttttgttaatacgaaaaagccgaaaacgttattatttctatcatcggcaaatttaatgttaaatttgctccacatcattcagagggtatatgtatatttttctaatgcttcggtgaagcacatgcgaccatttgttgcgtcagaagtcagaacaaaaggaagattccatctaatggattataatcccacatgcccatttttaaaactagacaaaataacttataatagtcccataagagcattaagacaaactgaaccaaaaagaatcttaaatgctggtgaatctagtctcgaatgctgctgaacactcctgatattagaatttggcagaaaatattcatactaataacagtaaatacatttcaaaacaagaaaatagtagtagtaaataaaaacaattaatatgttattattgtttctcttagctgttaccattacattaacaatttacattcaatccttttaatgaaaaatattataaaatattaaaagaagttattagaaaaggagagggatatgggggggggagagcatggaggggtcccatgtgtgtttcgaaaagcagcaaacatgactttgtaaagtcttgttttagctagagttccaaaaaaaaaagagttcaaaaaaaaagaaaaaaaaaagtcttgttttagcgtccaaagcggtctcactcgccgttcagctgttaaattttcttgaaataaatcgctccacttgttttttatttttgtttctttcaacccacttgtattaataacaataatgatattgctaatcttgaacaaatcgtggattgtcttttcttccagtgatatcatccttagtcaaaacatctcaaattaaataaatatatagttgtgaattaaaaaaaaggtatagagtttataaaattagcatttgcgaatgtttttaaaagtttggcgtttgtatagatcactgtcagtgtcatgaatcattcggtgacagcaaatttgtatatgccaaaaaagagagttgatacttgccattctcgatattttgaattttttttcgtcgacatacaaactcaacattttcgaaatcttagattaaaaacaaaatggacaaaacgatacttattaatgaattatgtactagcgtctgaacattagtaacatagatataattataagaacaagaaacatatatgcacaaagcctcaaaccaaatttaaatagtgttacgaaacaagggatgtacttgttatgaaaaattaatgtataaccattcttagcattttttacctaacccttaaactttataaagaaaaatatgtaacatttcggtgtgtggtatataaaagtattttgaaaatcgatttgattatttctattattaaaatgtacttactttttttatcacacatttatttacaatttcagagttaaacatgattaagacaaagtagtgaaaatatagatgatctaccagaaaatcatttgctttctgagtgagatcaaaataaaggcaaaaataatgtgatgattgtatattaaacaattattttgaatttgaaaagaataactcatcgaatgaagcctatgaaccgagtaagcggtgaagcggttggaccgtcacaaaaattatgaccaacaagaaaataaaattgatattagtgaagggcaattgtcaataatagcaccttttgaagtttatgtctcaaaaatagcactagaaggagaaagtcacaaaaatgacattcattaaagggtaaaatatctataatacccttggtttaaaattaaataaacaaactaaaataaataaaaataaaaaaataaaaaaaataaaaataaaaaaaataaatttttttttatagtttcagattatatgttttcagattcgaaatttttataatttttttttaaaaaaaaattttaaatcttttttttatttttttttcagattttatttttataatttaaaaatactttttgaaactgtttttaaaatttttattttttattttattatttattatttataaaattttaaatcctaattccaaaaccccaccccttaactctaaaccctaatgtttggattaattaaccctaggggtataagtgtacattacctctttaatgaaacctatttttgtgactttgaaccttgagtgctactttgggaacaaaaacttggtttggtgctattctagtatttttctcattagtgaatgatatattttagaaagtaataaattggttccagatttcaatacatcaatgtttttctaatgctgacttgcttttcctctttttcttacatgataaggaaacgagagaggtcgaagaaacaaagaaacttcctaagcatttagcaacgcatcttcaaaaaatctagtatttaccaagtaaaatcatacaaaataaggaaaaaaagaagaatatatttcccaatttggttttcaccggaaaaaaaaaactgtcttaacaaaatcttaatgcaccaaatcatataagaactttatttgaccattgtcattagttttttccttatggtaatggatcttcaaataatttaaattaaattaaaaaaaaaaagaaaagagaaacaaaaacgcggaaaggagagcccaaatcgtctccgtctgtcattgtcgcagtctctaaaagagaaaaacaaatcgcaacgcttctctctctctccctatcgtttgaatcagtctctcaagatccaccaccaccacacatgctactatgagcctccgccaccgcaccgtcccctctcaacccggacggcccccggcggcgggcgcaatcgaggacgagccctacaacatcatccccgtcaacaacctcctcgccgaccacccctccctccgctaccccgaggtccgcgccgccgccgccgccctcaaaaccgtcggcgacctccgccgccccacctacgtccaatggcgcccccactacgacctcctcgactggctcgccctcttcttcggcttccagaaggacaacgtccgcaaccagcgcgagcacctcgtcctccacctcgccaacgcccagatgcgcctcaccccgccgccggacaacatcgattccctcgatcccgccgtcgtccgccgcttccgccgcaagctcctcggtaactactccagctggtgctcctacctcgggaggaagtccaacatctggatctcggatcggacccccgattcgcggcgggagcttctctacgtcggcctctacctcctcgtgtggggcgaggcggcgaatcttaggtttatgcctgagtgtatctgttacatcttccacaatatggcctcggagcttaacaagatcctcgaggattgcctcgacgagagcacggggcagccgtattctcctaagatcacgggggagaatagtttcctaaacggcgtcgttaagcctatctacgagactattaaagctgagattaacgagagcaagaacgggacggagccgcattgtaagtggaggaactatgatgatattaatgagtacttttggacggataggtgttttagtaaattgaaatggccgattgatttggggagcagtttcttcaagaacagcagaggtagcggcgttgggaagactggttttgtggagaggaggacgtttttttacctctacaggagctttgataggctttgggtgatgcttgctttgtttcttcaagctgctattatagttgcttgggaggagaagccgggtggagggtcggtgacgagtcagctctggaatgcgttgaagtcgacggatgttcaggtgaggcttttgactgtgttcttgacgtggagtgggatgaggttgttgcaggctgtgttggacgctggctcgcaacggtcgcttatttctagagagaccaaacggctgtttttcagaatgttgatgaaggttgtggctgctacggtttggatagtagcgtttattgttctctacacgaacatctggaagcagaggaagcaagataggcagtggtccagagccgcgaatgataagatttatcagttcctttacgctgtggtggctttcttggtccctgagatcctggctttggctctgtttatagtcccgtggataaggaactttctggaagagacgaattggaagatattctttgctttgacttggtggttccaggggaaaagctttgtgggtcgaggtttgagagaggggttggtggacaacatcaagtactcgactttctggatctttgtccttgcaacgaagtttacgttcagctacttcctgcaggttaagccaatgattaaaccctcgaagctgctatggaatttgaaggaggtggattatgagtggcatcagttctttggcgagagcaataggttttctgtcttgttattgtggctgccaatggtgttgatatacctgatggatatccaaatttggtacgcgatctattcttcgattgttggtgctgttgttgggctgtttgatcatctgggggagatcagggacatgggacagcttaggctgaggtttcagttctttgctagcgctattcagttcaacctaatgcctgaggaacaactcctgaatgctagaggatttggtaacaagcttaaggacgccattcataggtaagtctattgaagcatgttactgatatttctatataatttactatatagtttgtctttacagtacaagctatgggattttagttttgttaaagcagatttctccgcaatgaactcgacttttcacattgtaataacattgagtttgtgtactttatgcagattgaagctgaggtatggattggggcggccgtttaagaaactcgagtccaatcaggttgaggctaacaagtttgcgctgatctggaatgagataatcctagctttcagagaggaggatatagtctctgatcgagaagtagagctgctggagctgccaaaaaattcctggaatgtgacagttatccgctggccgtgttttctgttgtgcaacgagcttttgcttgcactgagccaggcgaaagagctggttgacgcacctgataaatggctgtggcacaagatatgcaagaatgagtacaggcggtgtgctgtggttgaggcatatgaaagcatcaaacatctgttgctctcaatcatcaaaattgacaccgaagaacataaaattgttacaattttctttcagatgattgaggtgtctattcagggtgagcagttcaccaagaccttcaaagtggaccttttgccaaagatttatgagacactacagaagttggttgggctgttgaatgatgagaaagtggatgttgggcgagtggtgaatggtctgcagtctatttatgagattgcaacacgacagttcttcctagaaaagaagacgactgaacagctatctactgaggggttaactcctcatgatccagcctcaaagttactgtttcagaatgctgttaggcttcccgatgcaagcaatgaagacttcttccggcaggttaggcggttacacacaattctcacttctagggactctatgcacagcgtccctgtgaatctagaggcgagacggcggattgccttcttcagcaattcgctcttcatgaacttgcctcatgcacctcaggtggagaaaatgttggcgttcagtgttatgactccatactacagcgaggaagttgtatacagcaaagaacagctccgaaatgagactgaggatgggatttcaaccttgtattacctgcagacgatttatgccgacgaatggaaaaattttaaggaacggatgcggagggaaggtataaagacagatgttgagttgtggacaaccaagctgagagagctcaggctttgggcttcctacagaggtcagactttggcacgtacagttcgaggaatgatgtactattacagggctcttaagatgcttgcttttcttgactctgcgtctgaaatggacattcgggaggatgctcaggagcttggttcaatgaggagttcgcagggaaatcgattggatggggtggacgatgtaaatgacggatcttctctaagcagagcaactagctctgtgagcatgctgtataaaggccatgagcatgggactgcattgatgaaattcacatatgtcgtggcgtgccagatctatgggtctcaaaaagcgaagaaggagcctcaggccgaggaaattctgtatcttatgaagcaaaacgaagccctccgtattgcatatgtggatgaggtgcatgcgggcagggaagagactgagtattactccgttctggtgaaatacgatcacacgttggagaaggaagtggagatattccgtgtgaagctacctggtccggtgaagctgggtgagggaaagccagagaaccagaatcatgcaatgatctttacccgcggtgatgctgttcagaccatagatatgaaccaggataattattttgaggaggctctcaagatgagaaatttgctccaggagtttaggcattaccatgggatcagaaagccaactattcttggtgtccgggagcacatcttcacgggatctgtctcgtctctggcttggttcatgtctgctcaggagacaagtttcgtcactcttggtcagcgtgttctagccaacccgctgaaggtcagaatgcattatggtcaccctgatgtatttgacagattctggttcttgagtcgaggtggcatcagcaaagcttctagagttataaatatcagtgaggacatcttcgccgggtttaattgcacattgcggggcggtaacgtcacccaccacgagtatattcaggtagggaaatgttcatcatttggatattctaactaatttatacatcgacaacaatactataattccactttttttttgttataacctttttgtgtgtgcatatatgtattcaggttgggaagggtcgagatgttggattgaatcaaatatcaatgtttgaggctaaggtagccagtgggaatggagagcaggttcttagccgagatgtgtacaggctgggtcataggctcgatttcttcagaatgttatcatttttctacacaacggtggggtttttcttcaacacgatgatggtcattctcactgtctacgctttcctctggggccgggtttatcttgctctgagcggtgttgagaagtccgctctagcagacagcacagacaccaacgcagcgcttgctgtgatattgaaccagcagttcatcattcagcttggtctcttcacagctctgccaatgattgtggaatggtctctcgaggaaggtttccttctcgcgatatggaacttcattcggatgcagattcagctttcctctgtcttctacacattctcaatggggaccagagctcactattttggccgaaccattctccacggtggagcaaagtacagagccactggacgtggatttgttgtcgagcacaagagtttcactgaaaactaccgtctatacgcacgcagtcactttgtgaaggccatcgagcttgggctgatcctcatagtctacgctacgcacagtcccatcgccaaagactcattgatctacatagccatgactctcaccagctggttcctcgtgatttcatggatactggccccttttgtgttcaacccgtcaggattcgactggcttaagacggtctatgacttcgaaggcttcatgaactggatctggtatcaaggcagaatctcaacgaagtccgaacagagctgggagatatggtggtatgaggaacaggaccacctgagaaccaccggtataccaggaagaatcatggagataatcttggaccttcggtttttcttcttccagtacgggattgtataccagctcaaaatcgcaaacggatcaaccagcattctcgtctacttactctcatggatatacatctttgcagtgtttgtgttcttcctagtgatccaatacgcccgtgacaagtactctgcgagaaaccacatacggtacaggctcgtccagttcctcctgatcgtgtttggtacactggtgattgttgctctcctcgagttcacgcatttcagcttcgtggatatcttcacgagtcttcttgcgttcgtcccaaccggctgggggatcttgctgatcgcacaggctttgaggcccgcgctgcagaagatcgggcttatctggaacgcggttatctcccttgctcggttgtatgacatactgttcgggataatcatcatggttcccgtagcgttcatgtcgtggatgcctgggtttcaatcgatgcaaacgaggatcttattcaatgaagcttttagcagagggcttcgtatcatgcagattgtcactgggaagaaatcaaaaggcgatgtcgaagttgaaaaaagaaggtaaagcttcttatttacccaaacatattttatgttctgtttgtttggaatcttaaattacaacaacactaatgcaaagcttttataacttgtgtaggtcttgaggtatatggtaatttaaaagttgctggtttgcggcgatgaggttagttttgtattcttataagttatgcttcttgtctgaataagaactcagacacccgtgttttgtcttcttcttattttaaaccaggtttttggagagctttggttgaggaagctatttgattagttcatcggttagtgggagaaacatatatatatacatttgtcagtactttgttagagtgtgtggaggtggggacgactctgattctgattccttatgtggttattgtctgaaacgttacagcattttgtgaagtaggcttttgtgcaagatttgatatctttctctcattgtagagctttagagcattttttagtatatgtttaatctttgattttctaatgtcatttgcattcatattcacatcttctctgcttcttcgttgtctgaaaccagttttcagatgcttatccattgtcca

SEQ ID NO: 7 - DNA, 6389 bp, Brassica juncea
  source 1..6389: mol_type = genomic DNA
tatcattgtcgcagtctctctcacagctaaaaggaaaacagatcgcaacgcttcaatatctctctctctccattgttttgaattgaatcagtctcctcacccttctccaagatccaccacaccacatgccactatgagcctccgccaccgcaccgtcccatctcaaaccggacggccgtcggcggcgggaatcgaggaggagccctacaacatcatccccgtcaacaacctcctcgccgaccatccttctctccgttaccccgaggtccgcgccgccgccgccgctctcaaaaccgtcggcgacctccgccgtcctccctacgtccaatggcgtcctcactacgatctcctcgactggctcgccctcttcttcggcttccagaaggataacgtccgcaaccagcgcgagcacatggtgctccacctcgccaacgctcagatgcgtctcacgccgccgccggataacatcgattccctcgattcgacggttgtccgtcgctttcgccggaaactcctcggaaactactcgagctggtgctcttatttagggaagaaatcgaacatctggatctcggatcggaaccctgattcgaggcgggagcttctctacgttggcctctacctcctcgtgtggggcgaggcggcgaatcttaggtttatgcctgagtgcatctgttacatcttccataacatggcctcggagcttaataagattctggaagattgcctcgatgagaacacggggcaaccctatctgcctactctctcgggggaaaacgctttcctaaacggcgtcgttaaacctatttacgaaactatcaaagctgagattgatgagagcaagaacgggacggagccgcattgtaagtggaggaactatgatgatattaatgagtatttctggacggataggtgtttcagtaaattgaagtggccgcttgatctgggaagcagtttcttcaagagtagtagagggaaaagcgttgggaaaaccggttttgtggagcgcaggaccttcttttacctctacaggagctttgataggctttgggtgatgcttgcgttgttccttcaggccgccattataattgcttgggaggaaaagccggatagagggtcggtgacagggcagctgtggaatgccttgaagtccagagatgtccaggtgaggcttttgacagttttcttgacgtggagtgggatgagactactgcaggctgtgctggacgctggttcgcaacggtctcttatttctagagagaccaaacggctctttttcagaatgttgatgaaggttgtagctgccacagtttggataatagcttttattgtactctacacgaacatctggaagcagaggaagcaagacagacagtggtccagagccgcgaatgacaagatctaccagttcctttacgctgtggtggccttcttgatccctgagatcctggctctggccctgtttataatcccgtggattaggaactttctggaagagagcaattggaaaatattctttgctttaacttggtggttccagggtaaaagctttgtgggtcgaggtttgagagagggtttggtggacaacatcaagtactcgacgttctggatcttcgtcctagcaacaaagttcacattcagctacttcctacaggttaagccaatgattaaaccctcgaagctgctgtggaacttaaaggaggtagattatgagtggcatcagttctttggcgagagcaataggttttctgtcttattattgtggctgccagtggtgttgatatacctgatggatatccaaatttggtacgcaatctattcttcgattgttggtgctgttgttgggctgtttgatcatctgggggagatcagggacatgggacagcttaggctgaggtttcagttctttgctagcgctattcagttcaacctgatgcctgaggaacaactcctgaatgctagaggctttggtaacaagcttaaggacgccatccataggtaagtctgtggaagcatgttactgatttcctttagagtttaatgtacagagtttgtctttacggtacaagctatataattttagttttgttatagcagattctcgtaacttgactttttaacattgttataaccttgagtttgtgtactttatgcagattgaagctgaggtatggatttgggcggccgtttaagaaactcgagtctaatcaggttgaggccaacaagtttgctttgatctggaatgagataatcttagcttttagagaagaggatattgtctctgatcgagaagtagagctactggagctgccaaagaattcctggaatgtgacagttatccgctggccgtgtttcctgctgtgtaacgagcttttgcttgcactgagccaggcgaaagagctggttgatgctcctgataaatggctgtggcacaagatatgcaagaatgagtacaggcgttgtgctgtggttgaggcatacgacagcatcaaacatctgttgctctcgatcatcaaaatcgacactgaagaacataaaatcatcacggttttctttcagatgattaaggtttctattcagggagagcagttcaccaagaccttcaaagtggatcttctgccaaagatttacgagacactacagaagttggttgggctgttgaatggtgaggaaccggatatcgggagagtggtgaatgttctgcagtctatatatgagattgcaacacgacagttctttatagaaaagaagacaactgaacagctatctactgaaggattaactcctcatgatccagcctcaaagttactgtttcagaacgctgttaggcttcccgatgcaagcaatgaagacttcttccggcaggtgaggcggttacacacaattctcacttctagggactctatgcacagcgtccctgtgaatctagaggcgagacgtcggattgccttcttcagcaattcgctcttcatgaacttgcctcatgcacctcaggtggaaaaaatgttggcgttcagtgttatgactccatactacagcgaggaagttgtatacagcaaagaacagctcaaaaatgagactgaggacgggatttcaaccttgtattacctgcagacgatttatgctgacgaatggaaaaattttaaggaacggatgcgtagggaaggtataaagacagatgatgagctgtatacaaccaagctgagagagctcaggctttgggcttcctacagaggtcagactttggcacgtacagttcgagggatgatgtactattacagggctcttaagatgcttgcttttcttgactctgcgtctgaaatggacattcgggaggatgctcaggagcttggttcaatgaggagttcgcagggaaatctgggcggtcgatcgaatggggttgacgatgtaaatgaccgatcttctctaagcagagctactagctccgtgagcatgctgtataaaggccatgagcatgggactgcattgatgaaattcacatatgtcgtggcgtgccagatctatgggtctcaaaaagcgaagaaggagcctcaggcagaggaaattctctatcttatgaagcaaaatgaagccctccgtattgcgtatgttgatgaggtgcatgcaggcaggggagagaccgaatattactcagttctggtgaaatacgatcacactttggagaaggaagtggagatattccgtgtgaagctacctggtcccgtgaagctgggtgagggaaagccagagaaccagaatcatgcaatgatctttacccgtggtgatgctgttcagaccatagatatgaaccaggataattattttgaggaggctctcaagatgagaaatttgctccaggagtttagacattatcatgggatcagaaaaccaactattcttggtgtccgagagcacatcttcactggctctgtctcgtctctggcttggttcatgtccgctcaggagactagtttcgtcactcttggtcagcgtgttcttgctaacccgctgaaggtcagaatgcattatggccaccccgatgtatttgacagattctggttcttgagtcgaggtggcatcagcaaagcgtccagagtcataaatatcagtgaggacatcttcgctgggtttaactgcacattgcggggcggtaacgtcacacaccacgagtatattcaggttggtaactattcattatttggttatttctaactaatttatactgcgacagcaatcctatatttccatatttcttttgttataaccatgcaacttaacgttcattgtatctgagtgtcaatgtaaatttcataacccctgtttttgtgcatatattcaggttgggaagggtcgagatgttggattgaatcaaatatcaatgtttgaggcgaaggtagccagtgggaacggagagcaggttcttagccgagatgtgtacaggctgggtcatagactcgatttcttcagaatgttatcatttttctacacaacggtcgggtttttcttcaacacgatgatggtcattcttaccgtttacgctttcctatggggccggatttatcttgctctgagcggtgttgagaagtccgctctagcagacagtacagacaccaacgcagcgcttgcggttatattgaaccagcagttcatcattcagctcggtctcttcacagcactgccaatgattgtggaatggtctctcgaggaaggtttccttcttgcgatatggaatttcatccggatgcagattcagctttcttctgtcttctacacattctcaatggggaccagagctcactattttggccgaaccatcctccacggtggagcaaagtacagagccactggacgtggatttgttgtcgagcacaagagtttcactgagaactacagactgtatgcacgcagtcactttgtgaaggccatcgagcttgggctgatcctcatagtctacgctacgcacagtcccatcgccaaagactcattgatctatatagccatgactctcaccagctggttcctcgtgatatcatggatactggctccatttgtgttcaacccgtcaggtttcgactggcttaagacggtctatgacttcgaaggtttcatgaactggatctggtatcaaggcagaatctcaacgaaatccgaacagagctgggagatatggtggtacgaggaacaggaccacctgagaaccaccggtataccaggaagaataatggagataatcttggaccttcggtttttcttcttccagtacgggattgtataccagctcaaaatcgcaaacggatcaaccagcattctcgtctacttgttgtcgtggatatacatctttgctgtgtttgtgttcttcctggtaatccaatacgcccgtgacaagtactctgcgaaaaaccacatacggtacaggcttgtccagttcctcctgatcgtgtttggtattctggtgatcgttgctctgctggagttcacgcatttcagcttcgtggatatcttcacgagtcttcttgcgttcgtcccaaccggctggggaatcttgctgatcgcacaggctttaaggcctatgctgcagaagttcaggcttatctggaacgctgttgtctcccttgctcggttatatgacatactgttggggatactcatcatggttcccgtagcattcatgtcatggatgcctgggtttcagtcaatgcaaacgaggatcttattcaatgaagcttttagcagagggcttcgtatcatgcagattgtcactgggaagaaatcaaaaggcgatgtctaagttgaaaatgaaagaaggtaaagctctttatttactcaaacatcttttatgttctgtttgtttggaatcctaagattacaacaaaaccaatgcaaagctttataacttgcgtaggtcataataaggtacatggtaatttaaagttgttggtttgcggcgttgagatcagttggaggttagttttctcgagacatggtattctataagttattgcttcttgtctgaataagaactcaaaagaaaaaccatgtgttgttgttgttgtcttcttcttaaagcaggtgtttggagagcttttgttgaggaatctggaagttgggttagatcatgggttagcgggtgaaatatatattgtcagtactttgttagtgtgtggaaatgggactctgattcttgttgtctgaaacgatacagcattttgtgaagtagggcttttgtgaaagatttgtttctctttctctctcagtgtagagctttagagcattttttagtatatgtttaatctttttattttctaatgtcatttgcattcatatctcacgtcttcctcgttggtctgaaaccggttttgagatgcttatccattgtccacttctctatg

SEQ ID NO: 8 - DNA, 9343 bp, Raphanus sativus
  source 1..9343: mol_type = genomic DNA
caaacgatcatagaaatgtacctgtaattagtcaactgttcaaagagcttaatgcattgctccttatcaatctgcccggttttctgtcataaacatgaaatagaatacaacgtaaacacatatgtttagctatgtggtaactaacagggagcttccctgtatatatctgtaggaggtaaaaatcattttgaatggagtaacttacgtctttgtctataagaccaaaggctttctccagcattcttctcttcatttgatccattccagatacttgctttgcgagctacaaaaataacaaatatgataccacgctagtgcggtcgtttttagttttaacatacaaaagatcaagttttgcaataaacctgttctttgaaactgtcataaacaacggcaagaatcaagttcgtgacaaagtagacgccaattagtacgtagagcacgaagaacaacgaagaccagcgtgaagacctatacaaaaaaaaaaaagaacaaatcagtcgcccaaaacactggatgtaactcatttcaccatataaacttacttgtaagcacgaatccagacatcaggattgttggatgttgtgaacaaaataaacatctggtaaagagttgcaccgtaagacgtgaagaccgtgagtccttgttgtgtatcctcaaacataacaaaagcaatccaactggcaaagagaaggaacagcatccatagagcctgtggagatattcaagaagtgaaagcatagaaggacaatcaatacgagttcatcactgttctgaaagtcaaactcgattgggaagaccactgaccaagatattcaagtatgtgccaagcattccagaaagaaggacgaggctgtctcgaagttccctgtacagaaattttaagaaacacagtttttagacaagaagataatataaaacaaaagtgcactttcatcgtaaatggcattttggattgagacaagttgatagaaaagaagcataaatatgtgttgataccttatgctgaggatgaatatgataactctgacatatggggcgattctaaaaggaagaaaatcgaaagccactggagacaagtatagaatgtcaacgagcacatcaacaatcaaaactaccacgcaagcaacctggaatcaggaagaagaagacatttattgtgttaaaagttatgaaatcaatatcttaagttctgtccaacattcaacaaatagaaaagaaaaaaaaaagaaacagtgaagaagacttatgtaggggagaataccttcactaggttaaggcggcttgtccaaaagatacgggacccttcataggagattgggaaaaaagtatgtacaagtagtataaccagggtaagtacctgtttggagaaagcaaaaatataagaatttcagtacaatgagaaaggatcctggctagtccgaacgataagataacgagtatataaatattagcatctacttaaacgagatcaactagttttcacaaaagtgtgtaatgaatctcaagccttgaactttcaagtggatgcatataagaatatgtcagtacagtgagaaaggatcctggctagtctagtcccaacaatatacaccatggaagataagacgagtatgttaataaaattaacaacttcttaaacgagatcaacgagtcttgaatctttcaaaagtgggttaatatgaatctggtgtgcctaattgcactaaaatctatttgcatctacagcgaaaatgaaaggccagctacttgcctcaaagataatggattctgcattggtcaagtagggtaactccccaaggtaatagtaatctctgtctttgcaagacggcgtaggcttattttcacaccacaatggttgctgcaaaaagacaggacaagaaagactgtcacacttttttctattttcttttaaaagtattttcttttgggggggttggggggggggcaaaaggttgctgacctcaaagaagttaatcaaaatgagagcgaaatagttgagagaccagatgagatctaatcgtgtgtagataaagtagtacttggcagactctccgaagctggactggtcaagtatttgctcaggtagacctataccatcttcagcctacatacatacagcaaaataaataaataaataaataaaaaggttctcaaaatcagcaaaaaagacaaagaaaccgaacattttctattgctatattcgtaacttattatcaggttaaagaaacaaatcattttgaacaaaaaaaaaaaaaaaagaaacaaatcatattctgagcaattattcagaataaacgatttggacttgtcagactgattctattcactgataaattgagaaatctccctcaagggagttatgccatcatgtcacagtcagtcggacaaagatccatgaattttatcagaacaagaagaagacaaatatagttagggagttgagagatagaaagagaaccagatcgacgagagcagcagctttctgaaaggtggtaccatgagcgatagcttctgatctacgaacaatccgatccgtaccaccaccacgactctctctacctatcaaaggatcttccattttttttttcttcaaaaggatcgaaatggaaagcagaagaccaaggaccttatgtagtacctttctttctttctttctgtcagtttattgttggctacgaattttaatataatgatacagatcacaagaagaagatagtgatgatggcccagaagtagttgaatttttaaaataaattaaattttccaagtgatattagatcttgccgacaaacacgttggggtccctttctcaatttgtaactttgtggatcaggatctaagtagattggtcaaaactgagagagtggtgttgtggtccgaataaaactgattaaagaataagactgcagagactaacaaacagaagaaacagatacatagagaagtggacaatggagaagcatctcaaaactggtttaacaagcagagaagaagatgtgaaatatgaatgcaaaatgacattagaaaatcaaagattaaacatatactaaaaagaaagtactctaaagctctacaatgagagagaaagagaagaaatctgaacacaaaagccctacttcacacaaaatgctgtaacgtttcagacaataacaccatacaagaatcagaatcagagtcttccccccttcccccacactctaacaaagtactgacaaatgtatatatatatgtttcccccactaaccgatgctctaaccagataaccaacttccaaattcgtccacaaaagctctccaaaaacctgtttcaagcacaagacagaacaaaagatgggtttatttgagttcttcttcgcacaccaaggataacttaaaaaaaattccatctctcgagaaaactaactaacctccaaatgatcaccatcgccgcaaaccaacagctttttcaagttaccatttaccatacgacctacacaagttataaagcttcgcattggtcttgttgtaaatcttaggattccaaaacaaacaggatattaaagatgtttcagtaaataaaagagctttaccttcttctttcttcaacttagacatcgccttttgatttcttcccagtgacaatctgcatgatacgaagccctctgctaaaggcttcattgaataagatccttgtttgcattgattgaaacccaggcatccacgacatgaacgctacgggaaccatgatgagtatcccgaacagtatgtcatataccctagcaagggagacaaccgcgttccagataagcctaaacttcgtcagcgctggccttaaagcctgtgcgatcagcaagattccccatccggttgggacgaacgcaagaagactcgtgaagatatccacgaagctgaaatgcgtgaactctagcagagcaacaatcaccagaataccaaacacgatcaggaggaactgaacgagcctgtaccgtatgtggtttctcgcagagtacttgtcacgggcgtactggattatcaggaagaacacaaacactgcaaagatgtatatccatgacaacaagtagacgagaatgctggttgatccgcttgcgattttgagctggtaaacaatcccgtactggaagaagaaaaaccgaaggtccaagattatctccacaactcttcctggtataccggtggttctcaggtggtcctgttcctcataccaccatatctcccagctctgttccgatttcgttgagattctgccttgataccagatccagttcatgaagccttcgaagtcatagaccgtcttaagccagtcgaatcctgacgggttgaacacaaatggggccagtatccatgatatcacgaggaaccaactggtgagagtcatggctatgtagatcaatgagtctttggcgatgggactgtgcgtagcatagactatgaggatcagcccgagctcgatggccttcacaaagtgactgcgtgcatagagtcggtagttctcggtgaaactcttgtgctcgacaacaaatccacgtccagtggctctgtactttgctccaccatggagaatggttcgaccaaaatagtgagctctggtccccattgagaatgtgtagaagacagaggaaagctgaatctgcatccgaatgaaattccatatagctagaaggaaaccctcctcgagagaccattccacaatcattggcagggcagtgaacagaccaagctgaacgatgaactgctggttcagtatcacagcaagcgctgcgttggtgtctgtactgtctgctagagcggacttctcaacaccgctcagcgcgagataaacccggccccagaggaaagcgtaaaccgtaagaatgaccatcatcgtgttgaagaaaaacccgaccgttgtgtagaaaaatgataacattctgaagaaatcgagcctatgacccagcctgtacacatctcggctaagaacctgctctccattcccactggctaccttagcctcaaacattgatatttgattcaatccaacatcccggcccttcccaacctgaatatatgtacaaaaaacaagggttatgcaatttacattgaagaaacaaggaacgttaagttgcatggttataacaaaaaaaaaaattgaactataggatttctgtcgcagtataaattagttagaaatatcaaaataatgaacattaccaacctgaatatactcgtggtgggtgacgttaccgcctcgcaatgtgcaattaaacccggcgaagatgtcttcactgatatttatgactctggaagctttgctgataccacctcgactcaagaaccagaatctgtcaaaaacatcggggtgaccataatgcattctgaccttaagcgggttggcaagaacacgctgaccaagagtgacaaaactagtctcctgagcagacatgaaccacgccagagacgagacagagcccgtgaagatgtgctcccggacaccgagaatagttggctttctgatcccatgagattttctaaactcctggagcaaatttctcatcttgagagcctcctcaaaataattatcctggttcatatctatggtctgaacagcatcaccacgggtaaagatcattgcatgattctggttctctggctttccctcacccagcttcaacggaccaggcagcttcacacggaatatctccacttccttctccaacgtgtgatcgtatttcaccagaactgagtaatactctgtctctcccctgcccgcatgcacctcatccacatacgcaatacggagggcttcattttgcttcataagatacagaatttcctctgcctgaggctccttctttgctttttgagacccatagatctggcacgccacgacatatgtgaatttcatcaatgcagtcccatgctcatggcctttatacagcatgctcacggagctagttgctctgcttagagaagatccaccatttacatcgtcaaccccatccagtcgaccatttccctgcgaactcctcattgaaccaagctcctgagcatcctcccgaatgtccatttcagacgcagagtcaagaaaagcaagcatcttaagagccctgtaatagtacatcattcctcgaactgtacgtgccaaagtctgacctctgtaggaagcccaaagcctgagctctctcagcttggttgtccacaactcaacatctgtctttataccttccctacgcatccgttccttaaaatttttccattcgtcagcataaatcgtctgcaggtaatacaaggttgaaatcccatcctcagtctcgtttttgagctgttctttgctgtatacaacttcctcgctgtagtatggagtcagaacactgaacgccaacattttctccacctgaggtgcatgaggcaagttcatgaagagcgaattgctgaagaaggcaatccgccgtctcgcctctagattcacagggacgctgtgcatagagtccctagaagtgagaattgtgtgtaaccgcctaacctgccgaaagaagtcttcattgcttgcatcgggaagcctaacagcgttctgaaacagtaacttggaggctggatcatgaggagtcaacccctcattagatagctgttcagttgtcttcttctctatgaagaactgtcgtgttgcaatctcataaatcgactgcaaaacattcaccactcgctcaatatctggcttctcagcattcagtagcccaaccaacttctgaagtgtctcgtacatctttggcagaaggtccactttaaaggtcttggtgaactgctcaccctgaatagagacctcaatcatctggaagaagatcgtaatgattttatgttcttcggtgtcaatcttgatgattgagagcagcagatgtttgatgctgtcatatgcctcaacaacagcacaacgcctgtactcattcttgcatatcttgtgccacagccatttatcaggagcatcaaccagctctttcgcctggctcagtgcaagtaaaagctcgttgcacagcaggaaacacggccagcggataactgacacattccacgaattctttggcagctccagtagctctacttctcgatcagagactatatcctcttctctgaaagctaagattatctcattccagatcaacgcaaacttgttggcttcaacctgattagactcgagtttcttaaacggccgcccaaatccatacctcagcttcaatctgcataaagtacacaaactcaaggttattattatgttgaaaactcgaattccaagcttgtaccgtaaagacaaactctgtatagtaaactataaatataggaatatgcttgcacagacttacctatgaatggcgtccttaagcttgttaccaaagcctctagcattcaggagttgttcctcaggcattaggttgaactgaatagcgctagcaaagaactgaaacctcagcctaagctgtcccatgtccctgatctcccccagatgatcaaacaacccaacaatagcaccaacaatcgacgaatagattgcgtaccaaatttggatatccatcaggtatatcaacaccactggcagccataataacaagacagaaaacctattgctctcgccaaagaactgatgccactcataatctacctcctttaagttccatagcagcttcgagggtttaatcattggtttaacctgcaggaagtagctgaaaatgaacttcgttgctaggacaaagatccagaacgccgagtacttgatgttgtccaccaaaccctctctcaaacctcgacccacaaagcttttaccctggaaccaccaagttaaagcaaagaatattttccaattgctctcttccagaaagttccttatccacgggattataaacagagccaaagccaggatctcagggaccaagaatgccaccacggcgtaaaggaattggtagatcttgtcattcgcggctctggaccactgtctatcttgcttcctctgcttccagatgttcgtgtagagaacaataaaggctataatccaaaccgtggcagctacaaccttcatcaacattctgaaaaacagccgtttggtctctctagaaatgaggggccgttgcgaaccagcgtccagcacagcctgcagtagtctcatcccactccacgtcaagaaaactgtcaaaagcctcacctgaacatctctcgacttcaaggcattccacatctgctttgccaccgaccctctatccggcttctcctcccaagcaactataatggcggcttgaaggaacaaagcaagcatcacccaaagcctatcaaagctcctgtagagataaaaaaacgtcctccgctccacaaaaccggtcttcccaacgctcttccctctactattcttgaagaaactgctccccaaatcaagcggccatttcaacttactgaaacacctatccgtccagaaatactcattaatatcatcatagttcctccacttacaatgcggctccgtcccgttcttgctctcattaatctcagctcggatcgtctcgtaaataggtttaacgacgccgtttaggaaagcgttctccccagagagagtaggcgtataaggctgccccgtgttctcgtcgaggcaatcctccaacaccttgttaagctccgaggccatattgtggaagatgtaacagacgcactcgggcatgaacctaagattcgcggcctcgccccacacgaggaggtagagaccgacgtagagaagctcccgcctcgaatcggggctccgatccgagatccagatgttcgatttcctcccgaggtaggagcaccagctggagtagttcccgaggagcttgcggcggaagcggcggacgacggcgggatcgagggagtcgatgttgtcggggggaggggagaggcgcatctgggcgttggcgaggtggaggacgacgtgctcgcgctggttgcggacgttgtccttctggaagccgaagaagagggcgagccagtcgaggaggtcgtagtgagggcgccattggacgaagggaggacggcggaggtctccgacggttttgagagcggcggcggcggcgcggacctcggggtagcggagggaagggtggtcggcgaggaggttgttgacggggatgatgttgtagggctcctcctcggttccccccgccgacggccgtccggtttgagatgggacggtgcggtggcggaggctcatagtagcatgtggtggatcttggagatggtgtgaaggaggaggagactgattcaaaggatgtggagagagagagagagagagagagagagagagagagagagagagattgaattgaagcgttgcgatatgtttttctcttttagct

SEQ ID NO: 9 - DNA, 23 bp, Brassica sp.
  source 1..23: mol_type = genomic DNA
acacgaacatctggaagcagagg

SEQ ID NO: 10 - DNA, 22 bp, Brassica sp.
  source 1..22: mol_type = genomic DNA
gaaagccaccacagcgtaaagg

SEQ ID NO: 11 - DNA, 23 bp, Arabidopsis thaliana
  source 1..23: mol_type = genomic DNA
acgaaaccgacgaacaaccgcgg

SEQ ID NO: 12 - DNA, 23 bp, Arabidopsis thaliana
  source 1..23: mol_type = genomic DNA
tattgattctctcgattccgcgg

SEQ ID NO: 13 - DNA, 23 bp, Arabidopsis thaliana
  source 1..23: mol_type = genomic DNA
gaacgccattgaacatacggcgg

SEQ ID NO: 14 - DNA, 23 bp, Arabidopsis thaliana
  source 1..23: mol_type = genomic DNA
gattgcctcgatgagaacaccgg

SEQ ID NO: 15 - DNA, 19 bp, Synthetic construct
  source 1..19: mol_type = other DNA
cgccgtctcagagctacaa

SEQ ID NO: 16 - DNA, 20 bp, Synthetic construct
  source 1..20: mol_type = other DNA
ggcggcttgaaggaacaaag

SEQ ID NO: 17 - DNA, 48 bp, Synthetic construct
  source 1..48: mol_type = other DNA
gtcgacctgcaggcatgcctgtcttaaatggacatttgtagtaacaaa

SEQ ID NO: 18 - DNA, 40 bp, Synthetic construct
  source 1..40: mol_type = other DNA
aagttctctcctttactgacatcgcctttttgatttcttc

SEQ ID NO: 19 - DNA, 20 bp, Synthetic construct
  source 1..20: mol_type = other DNA
cctgttccaaaggtcctgca

SEQ ID NO: 20 - DNA, 20 bp, Synthetic construct
  source 1..20: mol_type = other DNA
tgtcataccacttgtccgcc

SEQ ID NO: 21 - DNA, 38 bp, Synthetic construct
  source 1..38: mol_type = other DNA
tatgaccatgattacgcccaatttgcggccaagttcca

SEQ ID NO: 22 - DNA, 45 bp, Synthetic construct
  source 1..45: mol_type = other DNA
actagtcagatctaccattcaagaccttcttttttcaacttcgac

SEQ ID NO: 23 - DNA, 41 bp, Synthetic construct
  source 1..41: mol_type = other DNA
tatgaccatgattacgccgcttgtcttgcttgtttttgctc

SEQ ID NO: 24 - DNA, 46 bp, Synthetic construct
  source 1..46: mol_type = other DNA
actagtcagatctaccatttaagaccttcttttttcaacttcgaca

SEQ ID NO: 25 - DNA, 41 bp, Synthetic construct
  source 1..41: mol_type = other DNA
tatgaccatgattacgcccgataaggaaacgagagaggttg

SEQ ID NO: 26 - DNA, 42 bp, Synthetic construct
  source 1..42: mol_type = other DNA
actagtcagatctaccatgctgtaacgtttcagacaataacc

SEQ ID NO: 27 - DNA, 41 bp, Synthetic construct
  source 1..41: mol_type = other DNA
tatgaccatgattacgccgtgtgtttcggaaagcagcaaac

SEQ ID NO: 28 - DNA, 39 bp, Synthetic construct
  source 1..39: mol_type = other DNA
actagtcagatctaccatactgaaacccaggcatccacg

SEQ ID NO: 29 - DNA, 42 bp, Synthetic construct
  source 1..42: mol_type = other DNA
tatgaccatgattacgccgttaggtgacgactatatcagcat

SEQ ID NO: 30 - DNA, 43 bp, Synthetic construct
  source 1..43: mol_type = other DNA
actagtcagatctaccatgagagaaagatatcaaatcttgcac

SEQ ID NO: 31 - DNA, 42 bp, Synthetic construct
  source 1..42: mol_type = other DNA
tatgaccatgattacgccgctaatcttgaacaagtcctggat

SEQ ID NO: 32 - DNA, 42 bp, Synthetic construct
  source 1..42: mol_type = other DNA
actagtcagatctaccatggaagacgtgagatatgaatgcaa

SEQ ID NO: 33 - DNA, 42 bp, Synthetic construct
  source 1..42: mol_type = other DNA
tatgaccatgattacgccgtacctgtaattagtcaactgttc

SEQ ID NO: 34 - DNA, 40 bp, Synthetic construct
  source 1..40: mol_type = other DNA
actagtcagatctaccatcaccatctccaagatccaccac

SEQ ID NO: 35 - DNA, 19 bp, Synthetic construct
  source 1..19: mol_type = other DNA
gacgctggctcgcaacggt

SEQ ID NO: 36 - DNA, 20 bp, Synthetic construct
  source 1..20: mol_type = other DNA
cggccgccccaatccatacc

SEQ ID NO: 37 - DNA, 18 bp, Synthetic construct
  source 1..18: mol_type = other DNA
acgctgcctcgcaacggc

SEQ ID NO: 38 - DNA, 21 bp, Synthetic construct
  source 1..21: mol_type = other DNA
tggccgcccaagtccatacct

SEQ ID NO: 39 - DNA, 19 bp, Synthetic construct
  source 1..19: mol_type = other DNA
acgcctggctcgcaacggc

SEQ ID NO: 40 - DNA, 20 bp, Synthetic construct
  source 1..20: mol_type = other DNA
cggccgccccaatccatacc

(19) United States
(12) Patent Application Publication
(10) Pub. No.: US 2025/0171799 A1
(43) Pub. Date: May 29, 2025

(54) USE OF beta-1,3-GLUCAN SYNTHASE LIKE 5 IN IMPROVING CLUBROOT DISEASE RESISTANCE AND RELATED PRODUCT DEVELOPMENT IN CRUCIFEROUS CROPS

(71) Applicant: Institute of Oil Crops Chinese Academy of Agricultural Sciences, Wuhan City (CN)

(72) Inventors: Lijiang LIU, Wuhan City (CN); Shengyi LIU, Wuhan City (CN); Yi ZHANG, Wuhan City (CN); Yupo WU, Wuhan City (CN); Chuanji ZHAO, Wuhan City (CN); Xiaohui CHENG, Wuhan City (CN); Fan LIU, Wuhan City (CN); Li REN, Wuhan City (CN); Junyan HUANG, Wuhan City (CN); Xiaoling DUN, Wuhan City (CN)

(73) Assignee: Institute of Oil Crops Chinese Academy of Agricultural Sciences, Wuhan City (CN)

(21) Appl. No.: 18/814,578

(22) Filed: Aug. 25, 2024

(30) Foreign Application Priority Data
Nov. 23, 2023 (CN) 202311580841X

(51) Int. Cl. C12N 15/82; C12N 9/10

(52) U.S. Cl. CPC C12N 15/8279; C12N 9/1051; C12Y 204/01034

(57) ABSTRACT

The present disclosure provides the use of β-1,3-glucan synthase like 5 (GSL5) for the improvement of the clubroot resistance in cruciferous crops as well as related products development, belonging to the area of molecular engineering for plant breeding. GSL5 gene is highly conserved in cruciferous plants and mutation of GSL5 can confer a broad-spectrum and high clubroot resistance to the cruciferous crops, demonstrating that molecular engineering of GSL5 can achieve the improvement of the clubroot resistance in cruciferous crops. The present disclosure provides a key gene and techniques for the improvement of the clubroot disease resistance and durable prevention and control of the clubroot disease in cruciferous crops.

### SEQUENCE LISTING INCORPORATION BY REFERENCE

The instant application contains a Sequence Listing which has been submitted electronically in XML format and is hereby incorporated by reference in its entirety. The name of the text file containing the sequence listing is LOTUT_PRIORITY_TO_202311580841X.xml, has a file size of 108,492 bytes, and was created on Aug. 25, 2024.

### TECHNICAL FIELD

The present disclosure belongs to the technical field of plant breeding, and in particular, to a use of the gene β-1,3-glucan synthase like 5 (GSL5) gene in the improvement of the clubroot disease resistance and the development of related products in the cruciferous crops.

### BACKGROUND

Cruciferous crops include Brassica napus, Brassica rapa, Brassica oleracea, Brassica juncea, Raphanus sativus, etc., which are important sources of human edible oils, vegetables, feed and industrial oils, and one of the most important economic crops in the world.

The cruciferous clubroot disease is a devastating soil-borne disease caused by the protozoan Plasmodiophora brassicae, and can jeopardize almost all the cruciferous crops, resulting in root swelling, rot and plant death. The average yield loss is about 25-50% annually, and in some severe diseased fields, the yield loss is up to 100%. The P. brassicae can survive in the soil for more than 10 years in the infested fields and thus, these fields would be unsuitable for the cultivation of any susceptible cruciferous crop.

Exploiting the clubroot resistance genes for clubroot resistance breeding is the most economical and effective measure to prevent and control the clubroot disease. P. brassicae specialize in the virulence and could be divided into different physiological races. The presently used clubroot-resistant loci are mainly from the European fodder turnips (B. rapa ssp. rapifera), and all of them are dominant and race-specific. Consequently, the clubroot resistance of the developed resistant varieties are usually lost after 3-year plantation in the disease fields. Meantime, the breeding of clubroot-resistant varieties always takes a long time but are always in great demand. However, the resistance of broad-spectrum clubroot-resistant gene cannot be easily overcome by the diverse physiological races and thereby, is an ideal gene for clubroot resistance breeding to durably control this disease. Presently, the broad-spectrum clubroot resistance gene has not yet been discovered.

### SUMMARY

In the present disclosure, we provide a use of the cruciferous crop GSL5 gene in improving clubroot disease resistance and products development in the cruciferous crops. By molecular engineering of GSL5, we can achieve the improvement of broad-spectrum clubroot resistance in the cruciferous crops, thereby providing a key gene and associated techniques to durably and efficiently control the cruciferous clubroot disease.

To achieve the above-mentioned objective of the present disclosure, the present disclosure provides the following technical solutions:

Decrease or termination in GSL5 transcription or expression level or mutation or modification of the protein sequence to terminate or change GSL5 function by molecular engineering of GSL5 promoter regions, coding regions, and non-coding regions in the cruciferous crops.

Preferably, the molecular engineering includes the gene editing, EMS mutagenesis, radiation mutagenesis, homologous recombination, and T-DNA insertion.

Preferably, the cruciferous crops include but not are limited to Arabidopsis thaliana, Brassica napus, Brassica rapa, Brassica oleracea, Brassica juncea, and Raphanus sativus.

Preferably, the nucleotide sequence of the GSL5 genes include SEQ ID NO.1, SEQ ID NO.2, SEQ ID NO.3, SEQ ID NO.4, SEQ ID NO.5, SEQ ID NO.6, SEQ ID NO.7 and SEQ ID NO.8.

The present disclosure further provides a nucleotide sequence for gene editing of GSL5 which is shown in SEQ ID NO.9 or SEQ ID NO.10 and the cruciferous crops include Brassica napus, Brassica rapa and Brassica oleracea.

The present disclosure further provides a recombinant vector and strain for gene editing of cruciferous crop GSL5 genes.

Compared to the current techniques, the present disclosure has the following advantages:
(1) GSL5 is highly conserved in the cruciferous crops and knocking out of it can achieve the improvement of broad-spectrum clubroot resistance in the cruciferous crops, providing the first gene in improving the broad-spectrum clubroot resistance in cruciferous crops. Therefore, the gene GSL5 is expected to have huge economic and social benefits worldwide.
(2) Gene editing of GSL5 can significantly shorten the period of clubroot resistance breeding cycle, providing a new way to achieve the improvement of clubroot resistance breeding in cruciferous crops.
(3) The new germplasm of Arabidopsis thaliana, Brassica napus, Brassica rapa, Brassica oleracea with a broad-spectrum clubroot resistance have been generated, providing key germplasm resources for efficient and durable prevention and control of the clubroot disease in cruciferous crops.
(4) GSL5 has been identified as a key susceptible gene for P. brassicae infection and pathogenesis and knocking out of GSL5 is expected to block a key step during P. brassicae infection process, thereby conferring a broad-spectrum clubroot resistance. The present disclosure provides a new theoretic and technical support to achieve the improvement of the clubroot resistance in cruciferous crops.
(5) GSL5 is widely spread and highly conserved in cruciferous crops. Molecular engineering of only one GSL5 is expected to achieve the improvement of broad-spectrum clubroot resistance in cruciferous crops, thereby being of great significance to cruciferous crop breeding.

### BRIEF DESCRIPTION OF FIGURES

FIG. 1 shows that loss-of-function mutation of GSL5 confers a broad-spectrum resistance to the clubroot resistance in A. thaliana. A. the gene structure of GSL5 genetic structure. The mutation gsl5-1, also known as pmr4-1 results in the presence of a stop condon at the position 2220 while the mutation gsl5-2 results in a deletion of sequence at position 484-613; B and C. the mutants gls5-1 and gsl5-2 are highly resistant to the clubroot disease and have no significant impact on plant growth and development; D. GSL5 expression is significantly up-regulated during the secondary infection of P. brassicae in the root cortex; E. GSL5 mutation confers a broad-spectrum clubroot resistance.

FIG. 2 shows the highly conserved characteristic of GSL5 in cruciferous plants; A. the highly conserved protein sequence of GSL5 in the representative cruciferous plants. B and C. the GSL5 from five representatives of cruciferous crops can functionally complement the Arabidopsis gsl5 mutant to restore its susceptibility.

FIG. 3 shows that gene editing of GSL5 in susceptible B. napus, B. rapa and B. oleracea can significantly improve the clubroot resistance; A and B. clubroot resistance test of the homozygous gsl5 mutant plants of the B. napus, B. rapa and B. oleracea; C. the edited target sequence of GSL5 the B. napus, B. rapa and B. oleracea.

FIG. 4 shows that B. napus mutant gsl5 has a broad-spectrum clubroot resistance without growth and development penalties. A: B. napus gsl5 homozygous mutant plants have a broad-spectrum high resistance to different P. brassicae isolates. B: the mutation of gsl5 has no obvious effect on plant growth and development of B. napus.

### DETAILED DESCRIPTION OF EMBODIMENTS

The present disclosure provides a use of the GSL5 (Glucan Synthase-Like 5 or Callose synthase 12, CalS12) in improving clubroot disease resistance of the cruciferous crop. Decrease or termination in GSL5 transcription or expression level or mutation or modification of the protein sequence to terminate or change GSL5 function by molecular engineering of GSL5 promoter regions, coding regions, and non-coding regions in the cruciferous crops could confer a broad-spectrum clubroot resistance in cruciferous crops.

The preferred molecular engineering includes but is not limited to the gene editing, EMS mutagenesis, radiation mutagenesis, homologous recombination, and T-DNA insertion.

Preferably, the cruciferous crops include but not are limited to Arabidopsis thaliana, Brassica napus, Brassica rapa, Brassica oleracea, Brassica juncea, and Raphanus sativus. Preferably, the nucleotide sequence of the GSL5 genes include SEQ ID NO.1, SEQ ID NO.2, SEQ ID NO.3, SEQ ID NO.4, SEQ ID NO.5, SEQ ID NO.6, SEQ ID NO.7 and SEQ ID NO.8. The detailed descriptions are in the following: SEQ ID NO.1 is the genomic sequence of A. thaliana GSL5; SEQ ID NO.2 is the genomic sequence of the subgenome A GSL5 of the B. napus; SEQ ID NO.3 is the genomic sequence of the subgenome C GSL5 of the B. napus; SEQ ID NO.4 is the genomic sequence of B. rapa GSL5; SEQ ID NO.5 is the genomic sequence of B. oleracea GSL5; SEQ ID NO.6 is the genomic sequence of the subgenome A GSL5 of the B. juncea; SEQ ID NO.7 is the genomic sequence of the subgenome BGSL5 of the B. juncea; and SEQ ID NO.8 is the genomic sequence of R. sativus GSL5. In view of the highly conserved characteristics of GSL5 in the cruciferous plants, the GSL5 genes of other cruciferous plants that were not mentioned should also be listed into the protective scope of the present disclosure.

The present disclosure further provides a nucleotide sequence for gene editing of the cruciferous crop GSL5 which is shown in SEQ ID NO.9 or SEQ ID NO.10. The cruciferous crop includes B. napus, B. rapa and B. oleracea crops. The GSL5 target sequences of B. napus, B. rapa and B. oleracea are completely identical and contain a length of 20 bases with the 3′ end containing an NGG motif, where N represents any one of the bases A, G, C, and T. In view of the highly conserved characteristics of GSL5 in the cruciferous plants, the consensus sequences of the GSL5 genes of all cruciferous crops containing above characteristics should be listed into the protective scope of the present disclosure.

The present disclosure further provides a recombinant vector containing the above nucleotide sequence. This recombinant vector can be transfected into the cruciferous crop to obtain clubroot resistant materials. The gene editing of GSL5 can result in insertion, deletion or mutation in the exon regions.

The present disclosure further provides a recombinant strain containing the recombinant vector.

The technical solutions provided by the present disclosure will be described in detail in several examples below that, however, should not be viewed to limit the protective scope of the present disclosure.

### Example 1: Mutation of A. thaliana GSL5 and Test of the Broad-Spectrum Clubroot Resistance

The A. thaliana mutant gsl5-1, also known as pmr4-1 (Stock No. CS3858), was ordered from the Arabidopsis thaliana Biological Resource Center, containing a mutation from G to A at the position 2220 (gene No. AT4G03550), resulting in the presence of stop codon and loss-of-function mutation of GSL5 protein. The A. thaliana mutant gsl5-2 was obtained by gene editing, and this mutant had a deletion in the first exon at the position 484-613, resulting in a frameshift mutation of the coding region and loss-of-function of GSL5.

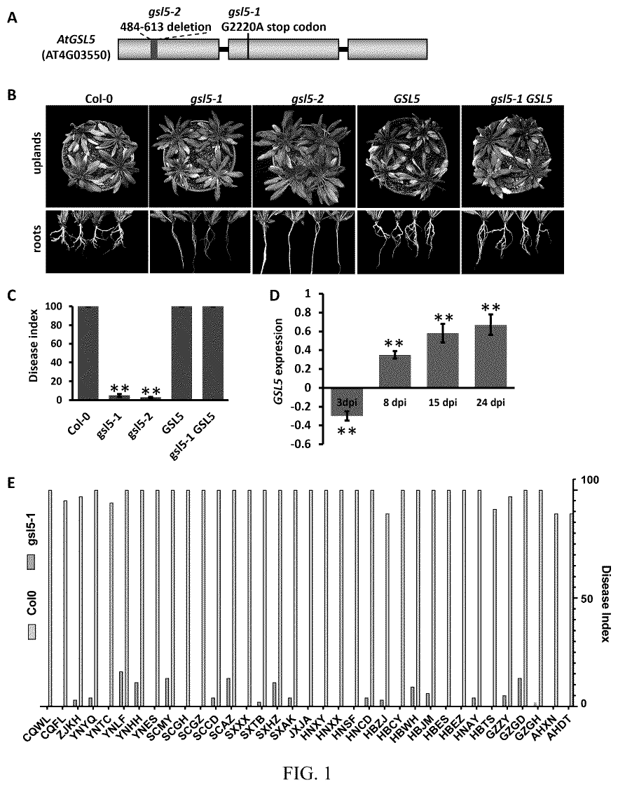

FIG. 1 shows that loss-of-function mutation of GSL5 confers a broad-spectrum resistance to the clubroot resistance in A. thaliana. Four target sites for gene editing were designed in the first exon of GSL5 with the target site sequences as follows:
sgRNA1: 5′-ACGAAACCGACGAACAACCGCGG-3′, shown in SEQ ID NO.11;
sgRNA2: 5′-TATTGATTCTCTCGATTCCGCGG-3′, shown in SEQ ID NO.12;
sgRNA3: 5′-GAACGCCATTGAACATACGGCGG-3′, shown in SEQ ID NO.13;
sgRNA4: 5′-GATTGCCTCGATGAGAACACCGG-3′, shown in SEQ ID NO.14;

Primers used for PCR identification of gene-edited progeny plants were:
GSL5-F1: 5′-CGCCGTCTCAGAGCTACAA-3′, shown in SEQ ID NO.15;
GSL5-R1: 5′-GGCGGCTTGAAGGAACAAAG-3′, shown in SEQ ID NO.16.

The GSL5 gene used to complement gsl5-1 was the full length of the genomic region with its native promoter (2500 bp upstream of the start codon). The genomic region of GSL5 was cloned from A. thaliana ecotype Col-0, the primers and sequencing were completed by Tsingke Biotechnology Co., Ltd. (Beijing). The primers used to clone the A. thaliana GSL5 promoter and gene fragment were:

AtGSL5F:
GTCGACCTGCAGGCATGCCTGTCTTAAATGGACATTTGTAGTAACAAA;
AtGSL5R:
AAGTTCTCTCCTTTACTGACATCGCCTTTTTGATTTCTTC;

As shown in SEQ ID NO.17 and SEQ ID NO.18, the targeted sequence is incorporated into the expression vector PBI121 by double enzyme digestion (enzyme cutting sites were HindIII and Kpn I) and homologous recombination. After verification by sequencing, the recombinant vector was transformed into the agrobacterium strain GV3101 subjected to transformation following the agrobacterium-mediated flower dipping method. The progeny plants were screened by using kanamycin resistance and positive plants were further identified by PCR to amplify a fragment of the kanamycin gene. The primers were in the follows:

KanR-jcF:
CCTGTTCCAAAGGTCCTGCA;
kanR-jcR:
TGTCATACCACTTGTCCGCC;

As shown in SEQ ID NO.19 and SEQ ID NO.20, the obtaining of the 500 bp PCR product demonstrated the transgenic plants overexpressing GSL5. By crossing the gsl5-1 plants with GSL5 overexpression plants, the progeny plants were identified with PCR to amplify the kanamycin gene (the primers were the same as the aforementioned) to screen the complemented gsl5-1 GSL5 plants. Clubroot resistance test of Col-0, gsl5-1, GSL5 and gsl5-1 GSL5 plants was carried out in a plant greenhouse. P. brassicae strains were collected from the diseased plants across China. Two-week-old seedlings were used for inoculation of different P. brassicae isolates with the resting spore suspension (108/plant) and the disease severity was investigated 30 days after inoculation according to the disease scale specified by the national industry standard of China [symbol] Technical Code of Practice for Evaluation of Clubroot Disease Resistance in Oilseed Rape (NY/T 3621-2020) [symbol] . The material with a disease index≤10 is considered to be highly resistant.

### Example 2: The Highly Conserved Characteristics of GSL5 in Cruciferous Plants

The homologs of Arabidopsis GSL5 were identified with the online collinearity analysis tool of the BRAD database (http://www.brassicadb.cn/#/syntenic-gene/) in the genomes of B. napus, B. rapa, B. oleracea, B. juncea, R. sativus. The genome of the allotetraploid crops B. napus and B. juncea contained two GSL5 homologs while the diploid crops B. rapa, B. oleracea and R. sativus contain only one GSL5 homolog. Multiple sequence alignment of the GSL5 from different cruciferous plants was carried out with an online tool COBALT (https://www.ncbi.nlm.nih.gov/tools/cobal-t/re_cobalt.cgi) and was virualized with the software Jalview. The genomic region of GSL5 from different cruciferous crops including its native promoter (2500 bp upstream of the start codon) was respectively cloned and connected to PBI121 expression vector. The agrobacterium containing the constructed vector was transfected into Arabidopsis gsl5-1 mutant and the progeny plants were identified using PCR. The resultant complemented plants were used for clubroot resistance test as the methods mentioned in Example 1. The primer sequences used to clone the genomic region of GSL5 from different cruciferous crops were shown in Table 1.

TABLE 1
The primer sequences used to amplify the genomic region of GSL5
from different cruciferous crops.

| | | | | Serial |
|---|---|---|---|---|
| | Amplified | | | number |
| Species | gene | Primer | Primer sequence | SEQ ID |
| Brassica napus | BnaA09.GSL5 | Forward | TATGACCATGATTACGCCCAATTTGCGGCCAAGTTC | NO. 21 |
| | | | CA | |
| | | Reverse | ACTAGTCAGATCTACCATTCAAGACCTTCTTTTTTC | NO. 22 |
| | | | AACTTCGAC | |
| | BnaC09.GSL5 | Forward | TATGACCATGATTACGCCGCTTGTCTTGCTTGTTTT | NO. 23 |
| | | | TGCTC | |
| | | Reverse | ACTAGTCAGATCTACCATTTAAGACCTTCTTTTTTC | NO. 24 |
| | | | AACTTCGACA | |
| Brassica rapa | BraGSL5 | Forward | TATGACCATGATTACGCCCGATAAGGAAACGAGA | NO. 25 |
| | | | GAGGTTG | |
| | | Reverse | ACTAGTCAGATCTACCATGCTGTAACGTTTCAGAC | NO. 26 |
| | | | AATAACC | |
| Brassica | BolGSL5 | Forward | TATGACCATGATTACGCCGTGTGTTTCGGAAAGCA | NO. 27 |
| oleracea | | | GCAAAC | |
| | | Reverse | ACTAGTCAGATCTACCATACTGAAACCCAGGCATC | NO. 28 |
| | | | CACG | |
| Brassica juncea | BjuA09.GSL5 | Forward | TATGACCATGATTACGCCGTTAGGTGACGACTATA | NO. 29 |
| | | | TCAGCAT | |
| | | Reverse | ACTAGTCAGATCTACCATGAGAGAAAGATATCAAAT | NO. 30 |
| | | | CTTGCAC | |
| | BjuB08.GSL5 | Forward | TATGACCATGATTACGCCGCTAATCTTGAACAAGT | NO. 31 |
| | | | CCTGGAT | |
| | | Reverse | ACTAGTCAGATCTACCATGGAAGACGTGAGATAT | NO. 32 |
| | | | GAATGCAA | |
| Raphanus | RsGSL5 | Forward | TATGACCATGATTACGCCGTACCTGTAATTAGTCA | NO. 33 |
| sativus | | | ACTGTTC | |
| | | Reverse | ACTAGTCAGATCTACCATCACCATCTCCAAGATCC | NO. 34 |
| | | | ACCAC | |

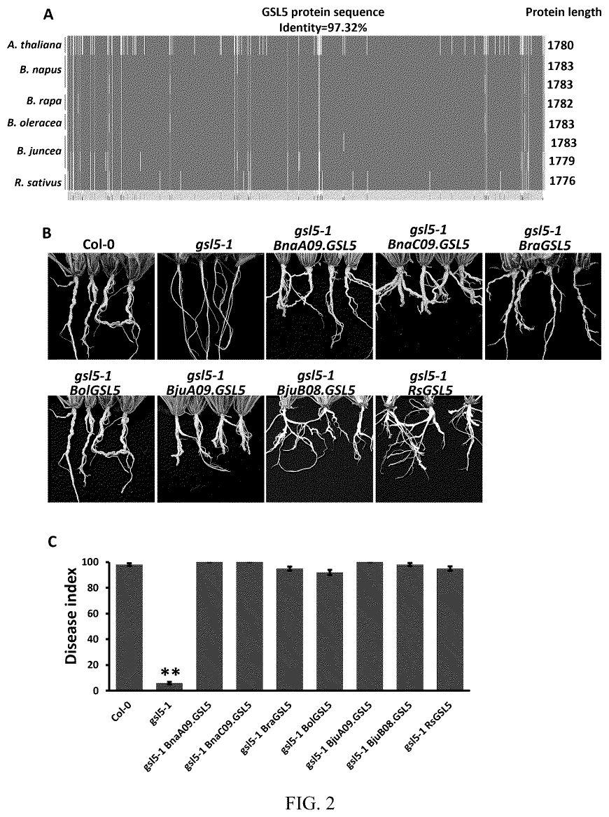

The results were shown in FIG. 2. A. the highly conserved protein sequence of GSL5 in the representative cruciferous plants. B and C. the GSL5 from five representatives of cruciferous crops can functionally complement the Arabidopsis gsl5 mutant to restore its susceptibility. B. napus (BnaA09.GSL5, BnaC09.GSL5), B. rapa (BraGSL5), B. oleracea (BolGSL5), B. juncea (BjuA09.GSL5, BjuB08.GSL5) and R. sativus (RsGSL5).

### Example 3: Knocking Out of GSL5 Genes of B. napus, B. Rapa and B. oleracea Achieved the Improvement of the Broad-Spectrum Clubroot Resistance Breeding

The cruciferous crops for gene editing of GSL5 include the B. napus spring variety Westar and hemi-winter variety Zhongshuang 11, the B. rapa ssp. chinensis variety Chinese cabbage F554, and the B. oleracea ssp. capitata variety HXF. The gene editing vector pYLCRISPR/Cas9 was used to knock out the GSL5 gene from above crops. Two target sites for gene editing were designed in the first exon of GSL5, and the target sequences were in the following:
sgRNA1: 5′-ACACGAACATCTGGAAGCAGAGG, shown in SEQ ID NO.9;
sgRNA2: 5′-GAAAGCCACCACAGCGTAAAGG, shown in SEQ ID NO.10;
The agrobacterium containing the constructed vectors were used to transfect the hypocotyl of above crops. The T1-generation transgenic plants were self-pollinated to generate the T2 population for screening the homozygous mutant gsl5. The primer sequences used to amplify the targeted editing sites of GSL5 from above crops were listed in the following:
   B. napus A Subgenome GSL5:
BnGSL5A-F: GACGCTGGCTCGCAACGGT, shown in SEQ ID NO.35;
BnGSL5A-R: CGGCCGCCCCAATCCATACC, shown in SEQ ID NO.36;
   B. napus C Subgenome GSL5:
BnGSL5C-F: ACGCTGCCTCGCAACGGC, shown in SEQ ID NO.37;
BnGSL5C-R: TGGCCGCCCAAGTCCATACCT, shown in SEQ ID NO.38;
The primers used to amplify the target site of GSL5 from B. rapa were the same as that used for amplification of the targeted sites from B. napus A subgenome.
   B. oleracea GSL5:
BoGLS5-F: ACGCCTGGCTCGCAACGGC, shown in SEQ ID NO.39;
BoGSL5-R: CGGCCGCCCCAATCCATACC, shown in SEQ ID NO.40;
Clubroot resistance test of the gsl5 mutant of above crops was performed as the methods mentioned in Example 1.

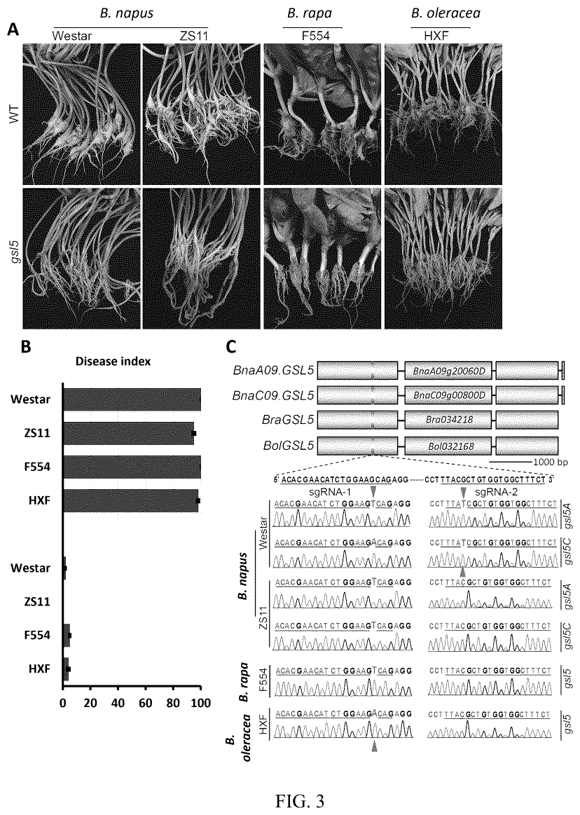

FIG. 3 shows that gene editing of GSL5 in susceptible B. napus, B. rapa and B. oleracea can significantly improve the clubroot resistance; A and B. clubroot resistance test of the homozygous gsl5 mutant plants of the B. napus, B. rapa and B. oleracea; C. the edited target sequence of GSL5 the B. napus, B. rapa and B. oleracea.

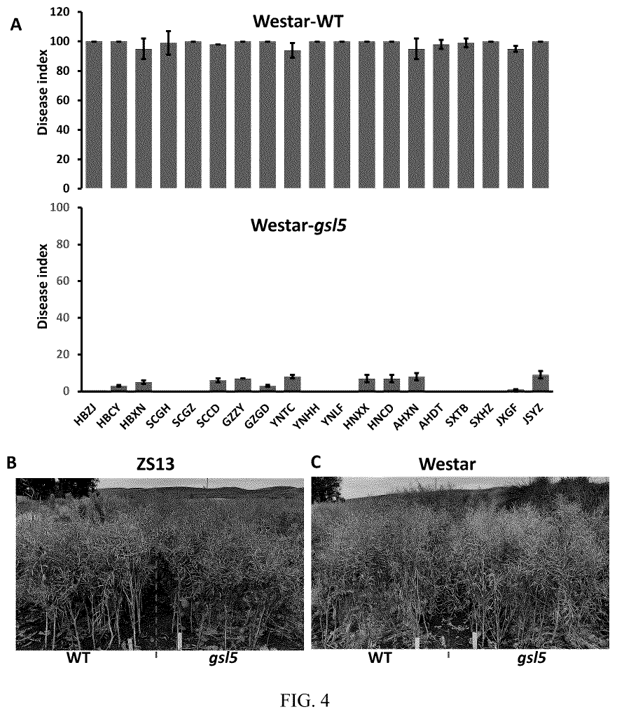

The gsl5 mutant of B. napus variety Westar were inoculated with different isolates of P. brassicae for clubroot resistance test. The results show that the gsl5 mutant plants have a broad-spectrum and high resistance to different isolates of P. brassicae (FIG. 4A). The gsl5 mutation had no obvious effect on the growth and development of the B. napus plants (FIGS. 4B and C).

The above clauses are only the preferable embodiments of the present disclosure. It should be noted that for those who possess the basic skills of the molecular engineering, further improvements and modifications of GSL5 in order to improve the clubroot resistance in cruciferous crops should be regarded as the protective scope of the present disclosure.

SEQ ID NO. 1
CTGTCTTAAATGGACATTTGTAGTAACAAAACCGACCATCAGCAAATTAA
CGTTAACGTTGCTCTAATCGTGTGTATTTTTCTAATGCTTGGTGAAGCAC
ATGCGACCATTTGTTGTGTCAGAACAAAAGGAAGATTCCACATTTAGAAT
TTAGATTATAATCCCACATGCCTATTATTTAAACATATGTATACTGTAAT
AAATTAGTCCCAGAAGAGCATTTCATCATACTGTATAATTAATCAAATTT
AAAAAACATTTGTGTTGAAGAGAAGATGACTAGATTCACTTGATTTTTTT
TAATACTGATTGGTCGTTTGACCAGTCAGGATCCAGACTGAAATATTCTC
AAAAGGTATGAACTATTTTAATGAAATATTTGAGCTGAGAATTTGCTTAT
GGGACATTCTATCCTTCTAAAGAAATCATTAGAAAATGGAGAAAAAGTTA
CTAGAAAAAGAGAGGGAAATGGGGCGAGAGTATGAAGAAAGGGTCCCATG
TGAATCTAATAATTGAAAAGCAAATATGAGATTTATTTTAGGGTTCTTGT
TTTAGCGTCCAAAGCGGTCGCAGTTGCCGTTCAGCGACTTTTCTCTGTTT
ATGAAATAAAAACGCTCCACTTGTTCTTTTCTTCTTTCAACCCACTTTGT
TATAACTATTAACAAATATTGCTTGGTCCAAATTTTATTATACTGAATTA
TTTTAGTTTTTTTGTATCATCATCCTCAATGTTAGTTTTGAAATCCTAGG
TCAGTAAGTCAATAACGAAAATGACAAGTTTAATTTTGTAATTGGAACTT
ACATGTCACGGTCTTTGTTTACGATCATTGCCACGAATTGTTTAGATACG
GGCTTTTTTTTTCTTTCCGTGTTGATGATTATCGTTTCTTCTTACATATC
TTGATCAATCACTAACATTTACATAACCTACAATTTTGTAATATAATTGC
AATACTCGAGATCTTCAAATGGTGATACTGTTAATAAAATTACGAACTTT
TAATCATCATCAAAATCTATAGAAAAAGATACATAGGACCAAAACAAGCC
TAAAACAAAGAGTTATAATTTACAAATAAATGCATGTATTTTGTTACTTT
TGTTAAGGATTTTCATATCAATGCATGCATAATGCATTCTCTTTTTATTT
TATTTATAATGCATTCTTTTATAATATTCGGATCCAAAATTATGAATTAA
TTCCAGATTGCCATAAATGTCTTTCTTTGTTTATGCTGACTTTTTTCTTT
TCTTAACTTATATAGTAAGTAAACCAGAAAGGTGGAAGATACAAAGCAAT
TTCATTGCATTAATAAACTTCAAATGTATTATCCAATAATCAAAATTTAT
CTAGTAATTCACAAAAAAAAAAAAATCTGTCTATCATTCAAAATTTCACC
AATTTGGATAAATATCTAGAAAATTTTAAAATACAATTCAATAACACTAT
TAACATATCAATCAATATATAACATTGCAAAAGTTTACTTGATCATTAGT
ATATTCATTTTAAGGTTAAAAGATCTTTAAGAAAAGGAAATTCAAATTAT
ATTGTTGGAAAAAAATACAAATCAAAGACAGCAAAAAAAGTAACAAAAAA
AAAAGGTAGAAGAAACTGAAACGCGGAAAGGAGGCAAAATCTTCTCGTCG
TCGTTGTCGCCGTCTTCAGAGCTACAAACGAAAAAACTCGCTTCCGTTTC
GATTTCTCCATTGTTATTGTTTCTTCAGTGAAGCTTTTTTCTTCGAGAAA
TTTCTAAGATCTACCACATGCTACTATGAGCCTCCGCCACCGCACCGTCC
CGCCGCAAACCGGACGGCCGTTGGCGGCGGAAGCTGTCGGAATCGAAGAG
GAGCCGTACAATATCATTCCCGTTAACAATCTCCTCGCCGACCATCCTTC
ACTCCGTTTTCCCGAGGTTCGTGCCGCCGCTGCTGCTCTTAAAACCGTTG
GAGACCTTCGTCGTCCGCCGTATGTTCAATGGCGTTCTCACTACGATCTC
CTCGACTGGCTCGCCTTGTTCTTCGGTTTCCAGAAAGATAACGTTCGTAA
CCAGCGTGAGCATATGGTGCTTCATCTCGCAAATGCTCAGATGCGTCTCT
CTCCGCCGCCGGATAATATTGATTCTCTCGATTCCGCGGTTGTTCGTCGG
TTTCGTCGGAAACTTCTCGCTAACTACTCTAGCTGGTGTTCGTATTTGGG
GAAAAAATCAAATATCTGGATCTCAGATCGGAACCCTGATTCGAGACGAG
AGCTTCTCTATGTTGGACTCTATCTTCTCATTTGGGGAGAGGCTGCGAAT
CTTCGGTTCATGCCTGAATGTATCTGTTACATCTTCCATAACATGGCCTC
TGAGCTCAACAAAATCTTAGAGGATTGCCTCGATGAGAACACCGGCCAAC
CTTACTTGCCTTCTCTCTCAGGCGAAAACGCTTTCTTAACCGGCGTCGTT
AAACCTATTTACGATACTATCCAAGCTGAGATTGATGAGAGCAAGAACGG
TACAGTTGCGCATTGTAAGTGGAGGAACTACGACGATATCAATGAGTACT
TCTGGACTGATCGGTGTTTCAGCAAATTGAAATGGCCGCTTGATTTGGGA
AGCAATTTCTTTAAGAGTAGAGGCAAAAGTGTAGGGAAAACTGGTTTCGT
GGAGCGCAGGACGTTCTTCTACCTTTACAGGAGTTTTGATCGACTTTGGG
TGATGCTAGCTTTGTTCCTTCAAGCCGCCATTATAGTAGCTTGGGAGGAA
AAGCCAGATACCTCGTCGGTAACAAGGCAGCTGTGGAATGCTCTGAAGGC
AAGAGATGTTCAGGTGAGACTATTGACCGTGTTCTTGACATGGAGTGGTA
TGCGACTCTTGCAGGCTGTGCTGGACGCGGCTTCACAATATCCCCTCGTT
TCCAGAGAGACCAAAAGGCATTTTTTCAGAATGCTGATGAAGGTTATAGC
TGCCGCAGTTTGGATTGTAGCTTTCACTGTCCTCTACACTAACATCTGGA
AGCAGAAGAGGCAAGACAGGCAGTGGTCCAATGCCGCGACGACTAAGATA
TACCAATTCCTTTACGCTGTGGGGGCCTTCTTGGTGCCCGAAATCCTGGC
TTTGGCTTTGTTTATTATCCCATGGATGAGAAACTTCCTGGAAGAGACCA
ATTGGAAAATATTCTTTGCTCTAACTTGGTGGTTTCAAGGCAAAAGCTTT
GTGGGTCGAGGTTTGAGAGAGGGTTTAGTGGACAACATCAAGTACTCGAC
TTTCTGGATCTTTGTCCTAGCTACAAAGTTTACATTTAGTTACTTCCTGC
AGGTTAAGCCAATGATTAAACCCTCAAAGCTGCTATGGAACTTAAAGGAT
GTCGATTATGAGTGGCATCAGTTTTATGGAGACAGCAATAGGTTTTCTGT
CGCATTGTTATGGTTGCCAGTTGTGTTGATATATCTGATGGATATCCAAA
TTTGGTACGCAATCTATTCTTCGATTGTTGGTGCTGTTGTTGGGCTGTTT
GATCATCTGGGGGAGATCAGGGACATGGGACAGCTGAGGCTAAGGTTTCA
ATTCTTTGCTAGTGCTATTCAATTCAACCTAATGCCTGAGGAACAACTCC
TGAATGCTAGAGGCTTTGGTAACAAGTTCAAGGACGGCATTCATAGGTAG
TCCGTGGAAGCATGCTACTAATATTCCTAAATAATTTTCTGTACAACGCT
TGACTTGACTGTACAAGCTGTGAAATTTTACTTTTGTTAACGCAGATGCT
GCATATAATTAAATTTTTCAATATTGTAATAACTTGAGGTTGTGTACTGT
ATGCAGATTGAAGCTAAGGTATGGATTTGGGAGGCCGTTTAAGAAACTTG
AGTCGAATCAGGTCGAGGCCAACAAGTTTGCGTTGATCTGGAACGAAATC
ATCTTAGCTTTCAGAGAAGAGGATATAGTTTCTGATCGTGAAGTAGAGCT
ACTGGAGCTGCCAAAGAATTCCTGGGATGTGACGGTTATTCGCTGGCCGT
GTTTCTTGTTGTGCAATGAGCTTTTGCTTGCACTGAGCCAGGCCAGAGAG
CTGATAGACGCACCTGATAAATGGCTGTGGCACAAAATATGCAAGAATGA
ATACAGGCGTTGTGCTGTAGTTGAGGCATATGACAGCATCAAACATCTAT
TGCTCTCAATCATCAAAGTTGACACTGAAGAACATTCGATAATTACGGTC
TTCTTTCAGATAATTAATCAGTCCATTCAGTCAGAGCAGTTCACCAAGAC
CTTTAGAGTGGACCTGCTGCCAAAAATTTATGAAACACTGCAGAAATTGG
TTGGGCTGGTAAATGATGAGGAAACAGATAGTGGGCGGGTGGTGAATGTT
CTGCAGTCTCTTTATGAGATTGCAACTCGACAGTTCTTTATAGAGAAGAA
GACAACTGAACAGCTATCTAATGAAGGTTTAACTCCTCGAGACCCAGCCT
CAAAGTTGCTGTTTCAAAATGCTATTAGGCTTCCTGATGCAAGCAATGAA
GACTTCTACCGGCAGGTTAGGCGTTTACACACGATTCTCACCTCTAGGGA
CTCTATGCACAGCGTCCCTGTGAATCTAGAGGCGAGACGGCGGATTGCTT
TCTTCAGTAATTCGCTTTTCATGAACATGCCTCATGCCCCTCAGGTTGAG
AAAATGATGGCGTTCAGTGTTCTGACTCCATATTACAGTGAGGAAGTTGT
ATACAGCAAAGAACAGCTCCGAAATGAGACTGAGGATGGGATTTCCACCC
TATACTACCTGCAGACAATTTATGCTGATGAATGGAAAAATTTCAAGGAA
CGGATGCATAGGGAAGGAATCAAGACAGATAGTGAGTTGTGGACAACCAA
GCTGAGAGACCTCAGGCTTTGGGCTTCCTACAGAGGTCAGACATTGGCAC
GTACAGTTCGTGGGATGATGTACTACTACCGGGCTCTTAAGATGCTCGCT
TTTCTTGACTCTGCGTCTGAAATGGACATTCGGGAGGGTGCTCAGGAGCT
TGGTTCAGTGAGGAATTTGCAGGGAGAACTGGGTGGTCAATCTGATGGGT
TTGTCTCTGAAAACGACCGATCTTCCTTAAGCAGAGCAAGTAGTTCCGTG
AGTACGCTGTATAAAGGCCATGAGTATGGGACTGCATTGATGAAATTCAC
ATATGTTGTGGCGTGTCAGATCTACGGGTCTCAAAAAGCAAAGAAAGAGC
CTCAGGCAGAGGAAATTCTGTATCTGATGAAGCAGAACGAAGCTCTCCGT
ATTGCATATGTGGATGAGGTGCCTGCGGGAAGAGGAGAGACTGATTATTA
CTCCGTTCTGGTGAAATACGATCACCAGTTGGAGAAGGAAGTGGAAATAT
TCCGTGTGAAGCTACCTGGTCCAGTGAAGCTGGGCGAGGGAAAGCCAGAG
AACCAGAATCATGCAATGATCTTTACCCGTGGTGATGCTGTTCAGACCAT
TGATATGAACCAAGACAGTTATTTTGAGGAAGCTCTCAAGATGAGAAATT
TGCTCCAGGAGTACAACCATTATCATGGTATCAGAAAACCAACTATTCTT
GGTGTCAGGGAGCATATCTTCACGGGATCAGTCTCGTCACTGGCGTGGTT
CATGTCTGCTCAGGAGACAAGTTTTGTCACTCTTGGTCAGCGTGTTCTTG
CAAACCCACTGAAGGTCAGAATGCATTATGGCCACCCTGATGTATTTGAC
AGATTCTGGTTCTTGAGTCGAGGCGGCATCAGTAAGGCTTCCAGAGTTAT
AAATATCAGTGAGGACATCTTTGCCGGGTTTAACTGCACGTTAAGGGGGG
GAAACGTCACCCACCACGAGTACATTCAGGTTGGGAAGGGTCCACAATTT
GGATTATTTCTAACTAACTATACTGCTACAACGTTTTTTTAACGTTTTTA
ACGTTTATTAATTATGCAATCTACTTTTGTTATAATTATGTAATTTAACG
TTTTTTAATCTTCTAAATTCAAAAAATTTGAGTAACCTTTGTCTTTATGC
ATTTTTCAGGTCGGGAAGGGACGGGATGTTGGATTGAATCAGATATCAAT
GTTTGAGGCTAAGGTAGCCAGTGGGAACGGAGAGCAGGTTCTCAGCCGAG
ATGTGTACCGGCTCGGGCACAGGCTTGATTTCTTCAGAATGTTATCATTT
TTCTACACAACTGTAGGGTTTTTCTTCAACACAATGATGGTCATTCTTAC
TGTTTACGCTTTCCTCTGGGGACGGGTTTATCTGGCTCTCAGCGGGGTTG
AGAAGTCCGCTCTAGCAGACAGTACGGACACCAACGCCGCGCTTGGGGTG
ATCCTGAACCAGCAGTTCATCATTCAGCTCGGTCTGTTCACTGCCCTGCC
AATGATTGTTGAATGGTCTCTCGAGGAGGGTTTCCTTCTAGCGATATGGA
ATTTCATTCGAATGCAGATTCAGCTTTCAGCTGTCTTCTACACATTCTCA
ATGGGGACCAGAGCTCACTATTTCGGTCGAACTATTCTCCATGGTGGGGC
CAAGTATAGAGCCACTGGACGTGGATTTGTTGTCGAGCACAAGGGATTCA
CTGAGAACTACCGACTGTATGCACGCAGTCACTTTGTGAAGGCCATCGAG
CTTGGGCTGATCCTCATAGTCTACGCTTCGCACAGTCCGATTGCCAAAGA
CTCGTTGATTTACATAGCCATGACTATCACCAGCTGGTTTCTTGTGATTT
CATGGATAATGGCCCCATTTGTGTTTAACCCATCAGGATTCGACTGGCTT
AAGACAGTCTATGACTTTGAAGACTTCATGAACTGGATCTGGTACCAAGG
CAGAATCTCAACGAAATCTGAACAAAGCTGGGAAAAATGGTGGTACGAGG
AACAGGACCACCTGAGAAACACCGGGAAGGCAGGATTATTTGTGGAGATC
ATCTTGGTCCTCCGGTTTTTCTTCTTCCAGTATGGGATTGTATACCAGCT
TAAAATTGCAAACGGATCCACCAGCCTTTTTGTCTACTTGTTCTCATGGA
TATACATCTTTGCTATATTTGTGCTCTTCCTAGTCATCCAATACGCCCGT
GACAAGTACTCGGCAAAAGCTCACATACGGTACAGGCTTGTCCAATTCCT
CCTGATCGTGCTTGCTATACTGGTGATTGTTGCTTTGCTCGAGTTCACGC
ATTTCAGCTTCATCGATATCTTCACAAGCCTTCTTGCATTCATCCCAACT
GGCTGGGGAATTCTGCTGATCGCACAGACTCAAAGGAAGTGGCTGAAGAA
TTACACTATTTTCTGGAATGCTGTTGTCTCTGTTGCTCGCATGTATGACA
TATTGTTTGGGATACTCATAATGGTTCCAGTAGCGTTCTTGTCATGGATG
CCTGGATTCCAGTCAATGCAAACGAGGATATTATTCAATGAAGCTTTTAG
CAGAGGACTTCGCATCATGCAGATTGTCACTGGGAAGAAATCAAAAGGCG
ATGTCTAAGTTTAAAAAACGGTAAAGCTCCTTGTTCTCAACACCTTATGT
TATGATCGTTTAAATCCTGGATTTCACACCAATGCGGGCTTTAAATTTGT
GTAGGTCTTAAGAAGTAAATGGTAGTTCAAATCCTATTGGTATGTGGCGA
AGGAATCAGTTGGAGGTTAGTTTTCCCGAAACAACCGAATTCGAAGTTTT
GTTTCGTCTAAAGAAAAACTCAGATGCTGATGATTTATCTTTGTATTTTA
AACAGGTTTTTGGAGAGTTTGGTTGGATGAGGAATCGGGAAGTTGGTTTG
ATTCGGTTAGATGGGTTTAGGGAGATATTTGATTGTCAGTGTGTGTGGAG
GGAACTCTGATTCTTGTATGGTTTTTGTTCTAAAGGTACAGCAATTTGTG
TAGTGAGGCTTTGTGTATTTGTTCTCCTTCTCTCATTATAGAGCTTTAGA
GCATTTTTAGTTTATATTCAGATTGTTATCTAATGTCATCTCGCAGAGCT
TTTGTTCACATTTCACATCTTTTCTTCTCCTTCTTAGTAGAGATCAGTTT
CAGATTAGATACTTGTCCATATTCCACTACTCTCGTCTATTATCGGTTTC
TGCTTGTCAATTTCTGGGTCCAAAATTGAAATA
SEQ ID NO. 2
CTTCCTTCTTTGCCGTCTCTCTTGAAGCTAAGTACACCGATATGGCCGCC
TTTAAGTATGTTAAATGCAAACACAAATCGATTATGTTCATCTCATAAGT
TATGTGAATATTTTTAGCTAGTAATTGGGTATAACATGTTTTGTAGGTAT
TTTGTGATCGCAAACGCTGTCGTGAGTGTTTACAGCTTTCTAGTTCTGTT
TCTTCCTAAGGAGAGTTTACTGTGGAAGTTCGTCGTCGTCTTGGATTTGG
TAATAATTGTTATTTGGTTTAGTACTTCTCTTCGTTCATTAATTCATTTG
CATTCTATCTCTAAGCATAGTTTTTTTCTAAAGGTAATATTATTGTTGAT
ATCTAGTAGTTTGATGTGTTCAGAGGAATAAAAGCATTTTTATTGGTGAA
ATACTGAAAAATAATAAAATGTTGATTATTATAGTTCAAATAAACAATTG
TTTTCTAGAAGAACATAAAGTAATCACAATATTATATTTTGCATGAGAGT
TTTTTCACGAAATTCTCATATTTCCTCTTATTATCATTTATCGTTGAGAT
ACTTTCTAAGAACTCTTTAATGGAGATGTTCTAGTAAATTAATCTTTGAA
CGAAAAGTTAAAACATACACTTTCAAAAAAAAAAAGTTAAAACAAATAAA
CTTGAATGTAATGTGTCTTAGTCACACGCTTATTATAGGTTGTTAGGTGA
CGACTATATCAGCATACATACGAATTGTCATTAACTTTTGACATTTTTGG
TGAATGTATTGGAGGTGATGACAATGCTACTAACGTCAAGCATATCAGCG
GCGTTAGCGGTGGCGCAAGTGGGAAAGAAAGGAAACGCAAACGCAGGTTG
GCTTCCAATTTGCGGCCAAGTTCCAAAGTTTTGCGATCAGGTCACCGGAG
CTCTCATTGCCGGCTTCGTCGCACTCGTCCTCTACGTCTTGTTACTCTTA
TACTCTCTTCACTCCGTCGTCGATCCTTTTCTTCTCCAGAAATCTTGAAT
CTAACTTCTTTTTTCACTTTCAGTATCGGTTAATATACGTTTTAATATCT
ATACTATAACGTCGTTTGTGTATTGTATTTATATGTTTTCTGTTTTAGGT
GTCGAGATTGTCATAGTATTTCATTTTTATGGACTAATTAACATTATAGA
CTGAACCACCTCTCATAAACTCTTACTCATAATTAAAAAAAAAAATCAAT
GCTTCATATATACATCTCTGTCAATCATTGGATTCGTATTTATTATAGTA
TCAACAAATTTGTGATACAAACCTAAACATGTGCAATATCTTTAGCTAAC
GAAATGGATATAGATACATCTTCCCTAGCTATGGAAATGAGTATTTGATC
AAAACGTAATTGCACTTGTTAGTTAGTCATTTCTCTCAGTGTAACAAAAC
ACCATTTATGAAAAAACACTGAGCATGGGCAAGTTTCGTCTTCACCTCCG
TTAATCTTTTGCATCGTAAAAACAATCGATGTTACAAAACATGTTTTGTT
TTAACGTTGTAAAAATATCAGGCTTGATTTGAATGTCATGTTATGCCGAC
TTTACTAGTTTGTGTAACCAACTAAGCTATTAAAATATGCGTAATTACAA
AACTGTCAGATTTAGTTTCACAAAATCTTTGTAACTTCGCGGTTTTTGAT
ATGGAAGTTGCTACAAAAACTGACTTGTGATATTATAATCTTTCGGCAAG
AAAAATATATAATACTAATTTCAGAAAGTATTCTGTTGAATTGTTGATCA
CCAAATACACATAAACCGGCAGATGTTGACATGTTTACGTGGGATCACTG
GGATAGAAGTTTTGCTTCTGTATTAGTATCGCGAACCGGTTTGTAGTAGC
GAAAATAACATTATTATTTTCAGATTTCATCATCGTCAAATTCAACGTTA
CATTTGAACAGGCTTGTAAAAAGTGGACATGTGGCATGAGTAAAAGATAA
AGAAAATTGTGAGCCTTTTTGTTAATACGAAAAAGCCGAAAACGTTATTA
TTTCTATCATCGGCAAATTTAATGTTAAATTTGCTCCACATCATTCAGAG
GGTATATGTATATTTTTCTAATGCTTCGGTGAAGCACATGCGACCATTTG
TTGCGTCAGAAGTCAGAACAAAAGGAAGATTCCATCTAATGGATTATAAT
CCCACATGCCCATTTTTAAAACTAGACAAAATAACTTATAATAGTCCCAT
AAGAGCATTAAGACAAACTGAACCAAAAAGAATCTTAAATGCTGGTGAAT
CTAGTCTCGAATGCTGCTGAACACTCCTGATATTAGAATTTGGCAGAAAA
TATTCATACTAATAACAGTAAATACATTTCAAAACAAGAAAATAGTAGTA
GTAAATAAAAACAATTAATATGTTATTATTGTTTCTCTTAGCTGTTACCA
TTACATTAACAATTTACATTCAATCCTTTTAATGAAAAATATTATAAAAT
ATTAAAAGAAGTTATTAGAAAAGGAGAGGGATATGGGGGGGGGGGGGGGA
GAGCATGGAGGGGTCCCATGTGTGTTTCGAAAAGCAGCAAACATGACTTT
GTAAAGTCTTGTTTTAGCTAGAGTTCCAAAAAAAAAAGAGTTCAAAAAAA
AAGAAAAAAAAAAGTCTTGTTTTAGCGTCCAAAGCGGTCTCACTCGCCGT
TCAGCTGTTAAATTTTCTTGAAATAAATCGCTCCACTTGTTTTTTATTTT
TGTTTCTTTCAACCCACTTGTATTAATAACAATAATGATATTGCTAATCT
TGAACAAATCGTGGATTGTCTTTTCTTCCAGTGATATCATCCTTAGTCAA
AACATCTCAAATTAAATAAATATATAGTTGTGAATTAAAAAAAAAGGTAT
AGAGTTTATAAAATTAGCATTTGCGAATGTTTTTAAAAGTTTGGCGTTTG
TATAGATCACTGTCAGTGTCATGAATCATTCGGTGACAGCAAATTTGTAT
ATGCCAAAAAAGAGAGTTGATACTTGCCATTCTCGATATTTTGAATTTTT
TTTCGTCGACATACAAACTCAACATTTTCGAAATCTTAGATTAAAAACAA
AATGGACAAAACGATACTTATTAATGAATTATGTACTAGCGTCTAAACAT
TAGTAACATAGATATAATTATAAGAACAAGAAACATATATGCACAAAGCC
TCAAACCAAATTTAAATAGTGTTACGAAACAAGGGATGTACTTGTTATGA
AAAATTAATGTATAACCATTCTTAGCATTTTTTACCTAACCCTTAAACTT
TATAAAGAAAAATATGTAACATTTCGGTGTGTGGTATATAAAAGTATTTT
GAAAATCGATTTGATTATTTCTATTATTAAAATGTACTTACTTTTTTTAT
CACACATTTATTTACAATTTCAGAGTTAAACATGATTAAGACAAAGTAGT
GAAAATATAGATGATCTACCAGAAAATCATTTGCTTTCTGAGTGAGATCA
AAATAAAGGCAAAAATAATGTGATGATTGTATATTAAACAATTATTTTGA
ATTTGAAAAGAATAACTCATCGAATGAAGCCTATGAACCGAGTAAGCGGT
GAAGCGGTTGGACCGTCACAAAAATTATGACCAACAAGAAAATAAAATTG
ATATTAGTGAATGATATATTTTGGAAAGTAATAAATTGGTTCCAGATTTC
AATACATCAATGTTTTTCTAATGCTGACTTGCTTTTCCTCTTTTTCTTAC
ATGATAAGGAAACGAGAGAGGTCGAAGAAACAAAGAAACTTCCTAAGCAT
TTAGCAACGCATCTTCAAAAAATCTAGTATTTACCAAGTAAAATCATACA
AAATAAGGAAAAAAAGAAGAATATATTTCCCAATTTGGTTTTCACCCGAA
AAAAAAACTGTCTTAACAAAATCTTAATGCACCAAATCATATAAGAACTT
TATTTGACCATTGTCATTAGTTTTTTCCTTATGGTAATGGATCTTCAAAT
AATTTAAATTAAATTAAAAAAAAAAGAAAAGAGAAACAAAAACGCGGAAA
GGAGAGCCCAAATCGTCTCCGTCTGTCATTGTCGCAGTCTCTAAAAGAGA
AAAACAAATCGCAACGCTTCTCTCTCTCTCCCTATCGTTTGAATCAGTCT
CTCAAGATCCACCACCACCACACATGCTACTATGAGCCTCCGCCACCGCA
CCGTCCCCTCTCAACCCGGACGGCCCCCGGCGGCGGGCGCAATCGAGGAC
GAGCCCTACAACATCATCCCCGTCAACAACCTCCTCGCCGACCACCCCTC
CCTCCGCTACCCCGAGGTCCGCGCCGCCGCCGCCGCCCTCAAAACCGTCG
GCGACCTCCGCCGCCCCACCTACGTCCAATGGCGCCCCCACTACGACCTC
CTCGACTGGCTCGCCCTCTTCTTCGGCTTCCAGAAGGACAACGTCCGCAA
CCAGCGCGAGCACCTCGTCCTCCACCTCGCCAACGCCCAGATGCGCCTCA
CCCCGCCGCCGGACAACATCGATTCCCTCGATCCCGCCGTCGTCCGCCGC
TTCCGCCGCAAGCTCCTCGGTAACTACTCCAGCTGGTGCTCCTACCTCGG
GAGGAAGTCCAACATCTGGATCTCGGATCGGAGCCCCGATTCGCGGCGGG
AGCTTCTCTACGTCGGCCTCTACCTCCTCGTCTGGGGCGAGGCGGCCAAT
CTTAGGTTTATGCCTGAGTGTATCTGTTACATCTTCCACAATATGGCCTC
GGAGCTTAACAAGATCTTAGAGGATTGCCTCGACGAGAGCACGGGGCAGC
CGTATTCTCCTAAGATCACGGGGGAGAATAGTTTCCTAAACGGCGTCGTT
AAGCCTATCTACGACACGATCAGAGCTGAGATTAATGAGAGCAAGAACGG
GACGGAGCCGCATTGTAAGTGGAGGAACTACGATGATATTAATGAGTACT
TCTGGACGGATAGGTGTTTTAGTAAATTGAAATGGCCGATTGATTTGGGG
AGCAGTTTCTTCAAGAACAGCAGAGGTAGCGGAGTTGGGAAGACAGGTTT
TGTGGAGAGGAGGACGTTTTTCTACCTCTACAGGAGCTTTGATAGGCTTT
GGGTGATGCTTGCTTTGTTTCTTCAAGCTGCTATTATAGTTGCTTGGGAG
GAGAAGCCGGGTGGAGGGTCGGTGACGAGTCAGCTCTGGAATGCGTTGAA
GTCGACGGATGTTCAGGTGAGGCTTTTGACTGTGTTCTTGACGTGGAGTG
GGATGAGGTTGTTGCAGGCTGTGTTGGACGCTGGCTCGCAACGGTCTCTT
ATTTCTAGAGAGACCAAACGGCTGTTTTTCAGAATGTTGATGAAGGTTGT
GGCTGCTACGGTTTGGATAGTAGCGTTTATTGTTCTCTACACGAACATCT
GGAAGCAGAGGAAGCAAGATAGGCAGTGGTCCAGAGCCGCGAATGATAAG
ATCTATCAGTTCCTTTACGCTGTGGTGGCTTTCTTGGTTCCTGAGATCCT
GGCTTTGGCTCTGTTTATAGTCCCGTGGATAAGGAACTTTCTGGAAGAGA
CGAATTGGAAGATATTCTTTGCTTTGACTTGGTGGTTCCAGGGGAAAAGC
TTTGTGGGTCGAGGTTTGAGAGAGGGGTTGGTGGACAACATCAAGTACTC
GACTTTCTGGATCTTTGTCCTTGCAACGAAGTTCACGTTCAGCTACTTCC
TGCAGGTTAAGCCAATGATTAAACCCTCGAAGCTGCTATGGAATTTGAAG
GAGGTGGATTATGAGTGGCATCAGTTCTTTGGCGAGAGCAATAGGTTTTC
TGTCTTGTTATTGTGGCTGCCAGTGGTGTTGATATACCTGATGGATATCC
AAATTTGGTACGCGATCTATTCTTCGATTGTTGGTGCTGTTGTTGGGCTG
TTTGATCATCTGGGGGAGATCAGGGACATGGGACAGCTTAGGCTGAGGTT
TCAGTTCTTTGCTAGCGCTATTCAGTTCAACCTAATGCCTGAGGAACAAC
TCCTGAATGCTAGAGGATTTGGTAACAAGCTTAAGGACGCCATTCATAGG
TAAGTCTATTGAAGCATGTTACTGATATTTCTATATAATTTACTATATAG
AGTTTGTCTTTAAAGTACAAGCTATAGTATTTTAGTTTTGTTAAAGCAGA
TTCTCCGCAATGAACTCGACTTTTTCACATTGTAATAACATTGAGTTTGT
GTACTTTATGCAGATTGAAGCTGAGGTATGGATTGGGGCGGCCGTTTAAG
AAACTCGAGTCCAATCAGGTTGAGGCTAACAAGTTTGCGCTGATCTGGAA
TGAGATAATCTTAGCTTTCAGAGAGGAGGATATAGTCTCTGATCGAGAAG
TAGAGCTGCTGGAGCTGCCAAAGAATTCCTGGAATGTGACAGTTATCCGC
TGGCCGTGTTTCCTGTTGTGCAACGAACTTTTGCTTGCACTGAGCCAGGC
GAAAGAGCTGGTTGACGCACCTGATAAATGGCTGTGGCACAAGATATGCA
AGAATGAGTACAGGCGGTGTGCTGTGGTTGAGGCATATGAAAGCATCAAA
CATCTGTTGCTCTCAATCATCAAAATTGACACTGAAGAACATAAAATTGT
TACAATTTTCTTTCAGATGATTGAGGTGTCTATTCAGGGTGAGCAGTTCA
CCAAGACCTTCAAAGTGGACCTTTTGCCAAAGATTTATGAGACACTGCAG
AAGTTGGTTGGGCTGTTGAATGATGAGAAAGTGGATGTTGGGCGAGTGGT
GAATGGTCTGCAGTCTATTTATGAGATTGCAACACGACAGTTCTTCCTAG
AAAAGAAGACGACTGAACAGCTATCTACTGAGGGGTTAACTCCTCATGAT
CCAGCCTCAAAGTTACTGTTTCAGAATGCTGTTAGGCTTCCCGATGCAAG
CAATGAAGACTTCTTCCGGCAGGTTAGGCGGTTACACACAATTCTCACTT
CTAGGGACTCTATGCACAGCGTCCCTGTGAATCTAGAGGCGAGACGGCGG
ATTGCCTTCTTCAGCAATTCGCTCTTCATGAACTTGCCTCATGCACCTCA
GGTGGAGAAAATGTTGGCGTTCAGTGTTATGACTCCATACTACAGCGAGG
AAGTTGTATACAGCAAAGAACAGCTCCGAAATGAGACTGAGGATGGGATT
TCAACCTTGTATTACCTGCAGACGATTTATGCCGACGAATGGAAAAATTT
TAAGGAACGGATGCGTAGGGAAGGTATAAAGACAGATGTTGAGTTGTGGA
CAACCAAGCTGAGAGAGCTCAGGCTTTGGGCTTCCTACAGAGGTCAGACT
TTGGCACGTACAATTCGAGGAATGATGTACTATTACAGGGCTCTTAAGAT
GCTTGCTTTTCTTGACTCTGCGTCTGAAATGGACATTCGGGAGGATGCTC
AGGAGCTTGGTTCAATGAGGAGTTCGCAGGGAAATCGATTGGATGGGGTG
GACGATGTAAATGACGGATCTTCTCTAAGCAGAGCAACTAGCTCTGTGAG
CATGCTGTATAAAGGCCATGAGCATGGGACTGCATTGATGAAATTCACAT
ATGTCGTGGCGTGCCAGATCTATGGGTCTCAAAAAGCGAAGAAGGAGCCT
CAGGCAGAGGAAATTCTGTATCTTATGAAGCAAAACGAAGCCCTCCGTAT
TGCATATGTGGATGAGGTGCATGCGGGCAGGGAAGAGACTGAGTATTACT
CCGTTCTGGTGAAATACGATCACACGTTGGAGAAGGAAGTGGAGATATTC
CGTGTGAAGCTACCTGGTCCGGTGAAGCTGGGTGAGGGAAAGCCAGAGAA
CCAGAATCATGCAATGATCTTTACCCGCGGTGATGCTGTTCAGACCATAG
ATATGAACCAGGATAATTATTTTGAGGAGGCTCTCAAAATGAGAAATTTA
CTCCAGGAGTTTAGGCATTATCATGGGATCAGAAAACCAACTATTCTTGG
TGTCAGAGAGCACATCTTCACGGGTTCTGTCTCGTCTCTGGCTTGGTTCA
TGTCTGCTCAGGAAACAAGTTTCGTCACTCTGGGTCAGCGTGTTCTAGCC
AACCCGCTGAAGGTCAGAATGCATTATGGTCACCCTGATGTATTTGACAG
ATTCTGGTTCTTGAGTCGAGGTGGCATCAGCAAAGCTTCTAGAGTTATAA
ATATCAGTGAGGACATCTTCGCCGGGTTTAATTGCACATTGCGGGGCGGT
AACGTCACCCACCACGAGTATATTCAGGTAGGGAAATGTTCATCATTTGG
ATATTCTAACTAATTTTATACATCGACAACAATACTATAATTCCACTTTT
TTTGTTATAACCTTTTTGTGTGTGCATATGTATTCAGGTTGGGAAGGGTC
GAGATGTTGGATTGAATCAAATATCAATGTTTGAGGCTAAGGTAGCCAGT
GGGAATGGAGAGCAGGTTCTTAGCCGAGATGTGTACAGGTTGGGTCATAG
GCTCGATTTCTTCAGAATGTTATCATTTTTCTACACAACGGTGGGGTTTT
TCTTCAACACGATGATGGTCATTCTCACTGTCTACGCTTTCCTCTGGGGC
CGGGTTTATCTTGCTCTGAGCGGTGTTGAGAAGTCCGCTCTAGCAGACAG
CACAGACACCAACGCAGCGCTTGCTGTGATATTGAACCAGCAATTCATCA
TTCAGCTTGGTCTCTTCACAGCTCTGCCAATGATTGTGGAATGGTCTCTC
GAGGAGGGTTTCCTTCTCGCGATATGGAACTTCATTCGGATGCAGATTCA
GCTTTCCTCTGTCTTCTACACATTCTCAATGGGGACCAGAGCTCACTATT
TTGGCCGAACCATTCTCCATGGTGGAGCAAAGTACAGAGCCACTGGGCGT
GGATTTGTTGTCGAGCACAAGAGCTTCACTGAGAATTACCGTCTATACGC
ACGCAGTCACTTTGTGAAGGCCATCGAGCTTGGGCTGATCCTCATAGTCT
ACGCTACGCACAGTCCCATCGCCAAAGACTCATTGATCTATATAGCTATG
ACTCTCACCAGCTGGTTCCTCGTGATATCATGGATACTGGCCCCTTTTGT
GTTCAACCCGTCAGGATTCGACTGGCTTAAGACGGTCTACGACTTCGAAG
GCTTCATGAACTGGATCTGGTATCAAGGCAGGATCTCAACGAAGTCCGAA
CAGAGCTGGGAGATATGGTGGTATGAGGAACAGGACCACCTGAGAACCAC
CGGTATACCAGGAAGAATCGTGGAGATAATCTTGGACCTTCGGTTTTTCT
TCTTCCAGTACGGGATTGTATACCAGCTCAAAATCGCAAACGGATCAACC
AGCATTCTCGTCTACTTACTCTCATGGATATACATCTTCGCAGTGTTTGT
GTTCTTCCTAGTAATCCAATACGCCCGTGACAAGTACTCTGCGAGAAACC
ACATACGGTACAGGCTCGTTCAGTTCCTCCTGATCGTGTTTGGTACACTG
GTGATTGTTGCTCTCCTCGAGTTCACGCATTTCAGCTTCGTGGATATCTT
CACGAGTCTTCTTGCGTTCGTCCCAACCGGCTGGGGGATCTTGCTGATCG
CACAGGCGTTGAGGCCTGCGCTGCAGAAGATCGGGCTTATCTGGAACGCG
GTTATCTCCCTTGCTCGGTTATATGACATACTGTTCGGGATAGTCATCAT
GGTTCCCGTAGCGTTCATGTCGTGGATGCCTGGGTTTCAGTCGATGCAAA
CGAGGATCTTATTCAATGAAGCTTTTAGCAGAGGGCTTCGTATCATGCAG
ATTGTCACTGGGAAGAAATCAAAAGGCGATGTCGAAGTTGAAAAAAGAAG
GTAAAGCTTCTTATTTACCCAAACATCTTTTTATGTTCTGTTTGTTTGGA
ATCTTAAATTACAACAACACTAATGCAAAGCTTTTACAACTTGTGTAGGT
CTTGAGGTATATGGTAATTTAAAAGTTGCTGGTTTGCGGCGATGTGACCT
GTTGGAGGTTAGTTTTGTATTCTTACAAGTTATGCTTCTTGTCTGAATAG
GAACTCAGACACCCGTGTTTTGTCTTCTTCTTATTAAACCAGGTTTTTGG
AGAGCTTTGGTTGAGGAAGCTATTCGATTAGATAAATCTTTTAGTGGGGG
AGACATATATATATACATTTGTCAGTACTTTGTTAGTGTGTGGAAGTGGG
GACGACTCTGATTCTGATTCCTTATGTGGTTATTGTCTGAAACGTTACAG
CATTTTGTGAAGTAGGCTTTTGTGCAAGATTTGATCTCTTTCTCTCATTG
TAGAGCTTTAGAGCATTTTTTAGTATATGTTTAATCTTTGATTTTCTAAT
GTCATTTGCATTCATATTCACATCTTCTTCGTTGGTCTTAAACCAGTTTT
CAGATGCTTATCCATTGTCCACTTCTCTATGTATCTGTTTCTTCTGTTTG
TTTTCAGTATTGTCTTTTATTCTTTAATCAGTTTTATTTGGACC
SEQ ID NO. 3
AGTGAGTTTGTTCTCCAGCGAGCCGGAGAAGGCATCAACGCAAACCTGGA
GGAACAGATTGACCGGTATGTTCATTAACGCTGCAAGCCAGCTTTGATGT
TAGCTGCGTAAAGATCCCTGCGTTTACATCAAAACATTACATTGTTTGCT
GTGACTAAGGTAACAGAGTAAACGTGTAAAATACTCTGGTGTTTTGGAGA
AAACAAACTTACATGGAGCCCTTAATTGCATCTATGGCAGCTTGTGCTGA
GAGGAAACCTTCAACGTCCACTTTTCCTTGTAGGTTACTAATATATGCAT
ATTTTATTTAAATCAATCCAAATATATGGGTAAAAAACTTTATTTGAGGA
TGTACTATATATATGAATGCCTAAGAGTATAATGTACAAAGGTGAAACTT
GCCTTGACATTGAAGGAGTAGAGGACTGTTTGCTCCATGGTAGTCATGTA
AGTGTGAAGAATAGAGAGATGAAGATCCTCCAAAAAAGCAATAGTCTTAA
TAAGTTGTCCTGGTCTTCTTCTTGAAAGTTTCTTGATCATGGCATCAAAC
TCTAGCAGCTTCACCTCCACATCAGCCAAGCACGACTCGTTCTCAGCTGT
TTCTTCCCGGAGCCCTCCTCCTCCTCCTATACGACTCATGTGACTCTAGG
CATTGTAGAAGTTGCTCAAGCTCTCTTACAAACTCTATTCCACCTATGAT
CGACGCTTGATCTCTCTGTTATATACATGCATATTAAAAAATATTTTAAA
ATAATGAATACAATCACGAAATGAAGATGTATATGTGTTATCATGATACC
CATTGAACGTAGGATCCAGACAAAAGAGACTTGAGGTTACGAAGATGCTC
GTTCATTTGCTTCCTATTCCTAGCGACCGCAATATGTCTCATTCGTTAGT
TTTCTACTTCTTCGCTTGTCTTGCTTGTTTTTGCTCTTCACCTCCTTTGT
CATATCATTCTTGTTCTCACTATCTCCTTCTTCTCCTCCAGTAAACCGAC
TTGTTATTGTCGTTAACGTCTAAGGATTCACCTAAAAAATTTGGTGCCTG
AAAGAAAATAAAGGTGAAGAAATAATTATTAAGATAATATAGGTCAATTG
ACTTGGAATCAAAATTTTCACCCTGCAAGTTGACATGGTTTCATACTAAT
GTGATACATTAAGACATAGTTTATGGTTTTAATGATTGTTACCTGATCCA
TCAGGCATTATACCAAAAGGACAGGCACGGAGGAACTATTCAGTTAGCCC
ATGCTCCTGCACAAATTTATCAATCAACTCATATCTAGAAGGATATATGA
GAAAAGAAGTTTCTGGTGTTATTAGGAGTTGTGAGGATGATGTTTATTAA
GAAATAAACTATATAAGCCTTTACATATAAATTACAAAATACCTTGATTC
TCATCCAAGATTCCGGTGTGGATCGATTTATCAATGACATATTTGCAAAA
AAAAATAATAGGGTTACCTGGAAGATGGGTGTTGGTGTTTGCTAGACCAA
GCAACTCATCTTTGCAGAACCTGAAACGTTCTTGAGGAATTGGTGAACCT
GGGTCAGTTAAGTCTTTCAAACCCGTCTCAATATTGAACCGGATCGACAC
AGGAGAGTCGGACAGCTTGAAGTCCGGACAGCTCAAAGCCGCTAACAGAG
TACACTTAACCAACAGTGAAAAGATGTCTGAATGTGTTTAAGCGGTAGAC
GTTCACGGTGGCTTAGGTGAGAGTGGCCTGCAAAGCATGAAAAATTGGAA
ACATAAACAAATGAAATAAATATTGCAATCATTAAAAATAAACTAAAGCG
CTTGTTAACTTTTTCATAATCAAAAGACATAAGCATATGGATGTCTCTAC
ACGGACTATTTGAATTTTAGGTATGAATAAAATATACGAAAAAAGTAAAA
AAGAATCAACAATCACAACAATGATCATATCCCAAATATTTGAAGATAAC
AAAAACTTATGGAGAATAACGAAGACATGAATGTATTGATCTCCGGTGAA
GAACTATTAGGTTTGAAAGTTTCATAACCAAAACACAACAACGCGAGAAA
GGATTAAAAAACAAAACAAAACTACATAGTCAAGCCGGATTTACCTTGGC
GTCCATGAAGAGAATGTTGACCCTCTCTGTCGCGCTTAACATTCCTATCT
TCCCAGAACCTAAGCAAACGGACCTGGGCGGTGGAGGTGCAGCGGCTAGC
TTTCAAATCGGCGAGAAAGACGGAGGAATTAGCCATTATCGACTTTGATT
TCTTAGAAAACAGTTTATGGGAATGTAGTAGGATTGTGAGAATGATGTTT
CTGGGAAGAGCACATACCTTTTTGTAGGAGGAGCTTCACCGCTTGTAAGA
GAGATGAGAGTATTGAGTGAGAAATCGTTGAGCTTCATCGTGGAGGATGG
ATTTAAAAAGGGCTTTAAGAGTAAGAATCTGTAACAGGTAGATTGCTGGA
TCCGTAGGAAGAAGATGAAGCATCTCGATCAAAACCCTAGGGTATGATAT
TGCAACGGCGGAGAAGAGAATGAGATGAAACCATAGAATTAAAAACTCTG
CGTTTCAGGCATACGTCTTACTCTGCTTTTTAAAGATTGGGCTTTATAAA
CCAAAACAATAACAAAGAATGTAGCCCAAACGGAAACAGAATAAATGAAA
CGCGGTAAAGTGAACAGATGTCACATAATGATTGGCTGATTTAATTGTCC
TACGTGGACAGCTTCTCTGATGCTCATATAACCCTTTTAGTATAGGTTAG
ATGATATCATCCTTAGTCAAAAGTCAAAACATCTCAAATTAAATAAGTAT
AGAGTTGTGAATTTTTAAAAAAGTATAGAGTATATAAAATTAGCATTTGC
GAATGTTTTTAAAAGTTTGGCGTTTGTATGGATCACTGTCAGTGTCATGA
ATCATTCGGTGACTGCAAAATTTTATATGACAAAAAAAAAAGAGAGTTGA
TACTTACCATTCTCGATATTTTGAAATTTTTTTCGTCGTTAATACAAACT
CAACATTTTCGAAATCTTGGATTAAAAAAAAAAAATATGGACAAAACGGT
ACTTATTAATTAATTATGTACTAGCGTCTTGACATTATTAATATAGATAT
AATTATAAGAACAAGAAACGTATATGCACAAACCCTCAAACCAAATTTAA
ATAGTGTTAAAAAACAAGGGATGTACTTGTTATGAAAAATTAATATATAA
CCATTCTTAACATTTTTACCTAACCCTTAAACTTTATAAATAAAAATACG
TAATATCCCGATAGAAATATTTTAAAATTTTGATTTGATTATTTATGTTA
TCAAAGTGCATTTATTTTTTTTTGTCACGTATATATTTAAAATTTCAACA
TTAAAAGTGTGTAAGATGGAGTAGTGAAAATATAGACGACTTACCATAAA
ATTATTTGTGATATCATTTGAATAAGACTAAAACACGGACAAAAATAATA
TAGTGATTGCATTGTTATTAAACAATTATTTGGATTTGAAAAGAAATAAC
TCATTGAATGAAGCCTATGAGCCGAGTAAACGGACGTGGATAGCTTAAGC
GGTTGGACCGTACAAAATTTATGACCAACAAGAAAATGAAATTGATTTTT
TTTTTGACGAAAAAATGAAATTGATTTTAGAAAGTAACAAAATGGTTCCA
GATTTCAATACTTCAACGTTTTTTTATGCCGACTTTCTTTTCCTCTTTTA
CTTACATGATAAGGAAACGAGAGAGGTTGAAGAAACAAAGAAACTTCCTA
AGCATTTAGAAATGTATCTTCAAAAAATCTAGTATTTACCAAGTAAAATC
ATTCAAAATAAGGAAAAAAAGAAGAATCTATTTCCCAATTTGGTTTTCAC
CGAAAAAAAAATTGTCTTAATAAAATCTTAATGCACCAAATCATATAAGA
ACTTTATATGACCATTGTCATTAGTTTTTTCTTTATGGTAATGGATCTTC
AAATAATTTAAATTAAATTAAAAAAAAAAAAGAGAAACAAAAACGCGGAA
AGGAGAGCCCAAATCGTCTCCGTCTGTCATTGTCGCAGTCTCTCACAGCT
AAATAAAAGAGAAAAACAAATCGCAACGCTTCAATATCTCTCTCCCAATC
GTTTGAATCAGTCTTCCTCATCACAATCCCCCAAAGATCCACCACACATG
CTACTATGAGCCTCCGCCACCGCACCGTCCCCTCTCAACCCGGACGGCCC
CCGGCGGCGGGCGCAATCGACGACGAGCCCTACAACATCATCCCCGTCAA
CAACCTCCTCGCCGACCACCCCTCCCTCCGCTACCCCGAGGTCCGCGCCG
CCGCCGCCGCCCTCAAAACCGTCGGAGACCTCCGCCGCCCCACCTACGTC
CAATGGCGCCCCCACTACGACCTCCTCGACTGGCTCGCCCTCTTCTTCGG
CTTCCAGAAGGACAACGTCCGCAACCAGCGCGAGCACCTCGTCCTCCACC
TCGCCAACGCCCAGATGCGCCTCACGCCGCCGCCGGATAACATCGATTCC
CTCGATCCCGCCGTCGTCCGCCGTTTCCGCCGCAAGCTCCTCGGTAACTA
CTCGAGCTGGTGCTCGTACCTCGGGAGGAAGTCGAACATCTGGATCTCGG
ATCGGAACCCCGATTCGAGGCGGGAGCTTCTCTACGTCGGCCTCTACCTC
CTCGTGTGGGGGGAGGCGGCGAATCTTAGGTTTATGCCGGAGTGTGTCTG
TTACATCTTCCACAATATGGCCTCGGAGCTTAACAAGATCCTCGAGGATT
GCCTCGACGAGAGCACGGGGCAGCCGTACTCTCCTAGAATCACGGGGGAG
AATAGTTTCCTAAACGGCGTCGTTAAACCTATTTACGAGACGATCAAAGC
TGAGATTAACGAGAGCAAGAACGGGACGGAGCCGCATTGTAAGTGGAGGA
ACTATGATGATATTAATGAATACTTTTGGACGGATAGGTGTTTTAGTAAA
TTGAAATGGCCGATTGATTTGGGGAGCAGTTTCTTCAAGAGTAGTAGAGG
GAGAGGCGTTGGGAAGACAGGTTTTGTGGAGAGGAGGACGTTCTTTTACC
TCTACAGGAGCTTTGATAGGCTTTGGGTGATGCTTGCTTTGTTTCTTCAA
GCTGCTATTATAGTCGCTTGGGAGGAGAAGCCGGGTGGAGGGTCGGTGAG
GAGTCAGCTCTGGAATGCGTTGAAGTCGAGGGATGTTCGGGTGAGGCTTT
TGACTGTGTTCTTGACGTGGAGTGGGATGAGATTACTGCAGGCTGTGCTG
GACGCTGCCTCGCAACGGCCGCTTATTTCTAGAGAGACCAAGCGGCTGTT
TTTCAGAATGTTGATGAAGGTTGTAGCTGCTACGGTTTGGATAATTGCTT
TTATTGTTCTCTACACGAACATCTGGAAGCAGAGGAAGCAAGACAGGCAG
TGGTCCAGAGCCGCGAATGACAAGATCTATCAGTTCCTTTACGCTGTGGT
GGCTTTCTTGGTCCCTGAGATCCTGGCTTTGGCTCTGTTTATAGTCCCGT
GGATAAGGAACTTTCTGGAAGAGACCAATTGGAAGATATTCTTTGCTTTG
ACTTGGTGGTTCCAGGGTAAAAGCTTTGTGGGTCGAGGTTTGAGAGAGGG
GTTGGTGGACAACATCAAGTACTCGACTTTCTGGATCTTTGTCCTAGCAA
CGAAGTTCACGTTCAGCTACTTCCTTCAGGTTAAGCCAATGATTAAACCC
TCGAAGCTGCTATGGAATTTGAAGGAGGTGGATTATGAGTGGCATCAGTT
CTTTGGCAAGAGCAATAGGTTTTCTGTCTTGTTATTGTGGCTGCCAGTGG
TGTTGATATACCTGATGGATATCCAAATTTGGTACGCGATCTATTCTTCG
ATTGTTGGTGCTGTTGTTGGGCTGTTTGATCATCTGGGGGAGATCAGGGA
CATGGGACAGCTTAGGCTGAGGTTTCAGTTCTTTGCTAGCGCTATTCAGT
TCAACCTAATGCCTGAGGAACAACTCCTGAATGCTAGAGGATTTGGTAAC
AAGCTTAAGGACGCCATTCATAGGTAAGTCTATTGAAGCATGTTACTGAT
ATTCCTTTATAATTTACTGTACAGAGTTTGTCTTTACGGTACAAGTTATG
GAATTTTAGTTTTGTTAAAGCAGATTCTCTTAACTCGGCTTTTCAACATT
GTAATAACCTTGAGTTTGTGTACTTTATGCAGATTGAAGCTGAGGTATGG
ACTTGGGCGGCCATTTAAGAAACTCGAGTCTAATCAGGTTGAGGCTAACA
AGTTTGCGCTGATCTGGAATGAGATAATCTTAGCTTTCAGAGAAGAGGAT
ATAGTCTCTGATCGAGAAGTAGAGCTACTGGAGCTGCCAAAAAATTCCTG
GAATGTGACAGTTATCCGCTGGCCGTGTTTCCTGTTGTGCAACGAGCTTT
TGCTTGCACTGAGCCAGGCGAAAGAGCTGGTTGACGCACCTGATAAATGG
CTGTGGCACAAGATATGCAAGAACGAGTACAGGCGGTGTGCTGTGGTTGA
GGCATATGAAAGCATCAAACATCTGTTGCTCTCAATCATCAAAATTGACA
CCGAAGAACATAAAATTATTACAATTTTCTTTCAGATGATTGAGGTGTCT
ATTCAGGGTGAGCAGTTCACCAAGACCTTCAAAGTGGACCTATTGCCAAA
GATTTATGAGACGCTACAGAAGTTGGTTGGGCTGTTGAATGATGAGAAAG
TGGATGTTGGGCGAGTGGTGAATGGTCTGCAGTCTATTTATGAGATTGCA
ACACGACAGTTCTTCATAGAAAAGAAGACGACTGAACAGCTATCTACCGA
GGGGTTAACTCCTCATGATCCAGCCTCAAAGTTACTGTTTCAGAATGCTG
TTAGGCTTCCCGATGCAAGCAATGAAGACTTCTTCCGGCAGGTTAGGCGG
TTACACACAATTCTCACTTCTAGGGACTCTATGCACAGCGTCCCTGTGAA
TCTAGAGGCGAGACGGCGGATTGCCTTCTTCAGCAATTCGCTCTTCATGA
ACTTGCCTCATGCACCTCAGGTGGAGAAAATGTTGGCGTTCAGTGTTATG
ACTCCATACTACAGCGAGGAAGTTGTATACAGTAAAGAACAGCTCCGAAA
TGAGACTGAGGATGGGATTTCAACCTTGTATTACCTGCAGACGATTTATG
CCGACGAATGGAAAAATTTTAAGGAACGGATGCGTAGGGAAGGTATAAAG
ACAGATGTTGAGTTGTGGACAACCAAGCTGAGAGAGCTCAGGCTTTGGGC
TTCCTACAGAGGTCAGACTTTGGCACGTACAGTTCGAGGAATGATGTACT
ATTACAGGGCTCTTAAGATGCTTGCTTTTCTCGACTCTGCGTCTGAAATG
GACATTCGGGAGGATGCTCAGGAGCTTGGTTCAATGAGGAGTTCGCAGGG
AAATCGATTGGATGGTGTTGACGATGTAAATGACCGATCTTCTCTAAGCA
GAGCAACTAGCTCTGTGAGCATGCTGTATAAAGGCCATGAGCATGGGACT
GCATTGATGAAATTCACATATGTCGTGGCGTGCCAAATCTATGGGTCTCA
AAAAGCGAAGAAAGAGCCTCAGGCAGAGGAAATTCTGTATCTTATGAAGC
AAAACGAAGCCCTTCGTATTGCATATGTGGATGAGGTACATGCGGGCAGG
GGAGAGACTGAGTATTACTCCGTTCTGGTGAAATACGATCACACGTTGGA
GAGGGAAGTGGAGATATTCCGTGTGAAGCTACCTGGTCCGGTGAAGCTGG
GTGAGGGAAAGCCAGAGAACCAGAATCATGCAATGATCTTTACCCGTGGT
GATGCTGTTCAGACCATAGATATGAACCAGGATAATTATTTTGAGGAGGC
TCTCAAGATGAGAAATTTGCTCCAGGAGTTTAGGCATTATCATGGGATCA
GAAAACCAACTATTCTTGGTGTCAGAGAGCACATCTTCACGGGTTCTGTC
TCGTCTCTGGCTTGGTTCATGTCTGCTCAGGAGACAAGTTTCGTCACTCT
TGGTCAGCGTGTTCTAGCCAACCCGCTGAAGGTCAGAATGCATTATGGTC
ACCCTGATGTATTTGACAGATTCTGGTTCTTGAGTCGAGGTGGCATCAGC
AAAGCTTCTAGAGTTATAAATATCAGTGAGGACATCTTCGCCGGGTTTAA
TTGCACATTGCGGGGCGGTAACGTCACCCACCACGAGTATATTCAGGTAG
GGAAATGTTCATCGTTTGGATATTCTAACTAATTTATACATCGACAACAA
TACTATAATTCCACTTTTTTGTTATAACCTTTTTGTGTGTGCATATGTAT
TCAGGTTGGGAAGGGTCGAGATGTTGGATTGAATCAAATATCAATGTTTG
AGGCTAAGGTAGCCAGTGGGAATGGAGAGCAGGTTCTTAGCCGAGATGTG
TACAGGTTGGGTCATAGGCTCGATTTCTTCAGAATGTTATCATTTTTCTA
CACAACGGTGGGGTTTTTCTTCAACACGATGATGGTCATTCTCACTGTCT
ACGCTTTCCTCTGGGGCCGGGTTTATCTTGCTCTGAGCGGTGTTGAGAAG
TCCGCTCTAGCAGACAGCACAGACACCAACGCAGCGCTTGCTGTGATATT
GAACCAGCAGTTCATCATTCAGCTTGGTCTCTTCACAGCTCTGCCAATGA
TTGTGGAATGGTCTCTCGAGGAGGGTTTCCTTCTCGCGATATGGAACTTC
ATTCGGATGCAGATTCAGCTTTCTTCTGTCTTCTACACATTCTCAATGGG
GACCAGAGCTCACTATTTTGGCCGAACCATTCTCCACGGTGGAGCAAAGT
ACAGAGCCACTGGACGTGGATTTGTTGTCGAGCACAAGAGTTTCACTGAA
AACTACCGTCTATACGCGCGCAGTCACTTTGTGAAGGCCATCGAGCTTGG
GCTGATCCTCATAGTCTACGCTACGCACAGTCCCATCGCCAAAGACTCAT
TGATCTATATAGCCATGACTCTCACCAGCTGGTTCCTCGTGATTTCATGG
ATACTAGCCCCTTTTGTGTTCAACCCGTCAGGTTTCGACTGGCTTAAGAC
GGTCTACGACTTCGAAGGCTTCATGAACTGGATCTGGTATCAAGGCAGAA
TCTCAACGAAGTCCGAACAGAGCTGGGAGATATGGTGGTATGAGGAACAG
GACCACCTGAGAACCACCGGTCTACCAGGAAGAATCATGGAGATAATCTT
GGACCTTCGGTTTTTCTTCTTCCAGTACGGGATTGTATACCAGCTCAAAA
TCGCAAACGGATCAACCAGCATTCTCGTCTACTTACTCTCATGGATATAC
ATCTTCGCAGTGTTTGTGTTCTTCCTGGTAATCCAATACGCCCGTGACAA
GTACTCAGCGAGAAACCACATACGGTACAGGCTCGTTCAATTCCTCCTGA
TCGTGTTTGGTACACTGGTGATTGTTGCTCTCCTGGAGTTCACGCATTTC
AGCTTCGTGGATATCTTCACGAGTCTTCTTGCGTTCGTCCCAACCGGCTG
GGGAATCTTGCTGATCGCACAGGCTTTGAGGCCTGCGCTGCAGAAGATCG
GGCTTATCTGGAACGCGGTTATCTCCCTTGCTCGGTTATATGACATACTG
TTCGGGATAGTCATCATGGTCCCCGTAGCGTTCATGTCGTGGATGCCTGG
GTTTCAGTCGATGCAAACGAGGATCTTATTCAATGAAGCTTTTAGCAGAG
GGCTTCGTATCATGCAGATTGTCACTGGGAAGAAATCTAAAGGCGATGTC
GAAGTTGAAAAAAGAAGGTAAAGCTTTTATAACTTGTGTTCTGTTTATGT
TTGTTTGGAATCTTAAATTACAACAACACCAATGCAAAGCTTTTATAACT
TGTTTAGGTCTTAAGGTATATGGTAACTTAAAAGTCGTTGGTTTTGCGGC
GATGTGATCAGTTGGAGGTTAGTTTTCTCAAGACATGGAAGTTTTATAAG
TTATTGCTTCTTGTGTGAAGAAGAAGTCATATACATCCATGTTTTGTTTT
GTCTTCTTCTTAAAGCAGGTTTTTGGAGAGCTTTGGTTGAGGAATCCGGA
AGTTAGATAATCTTTTAGTGGGGGAGACATATATATACACATTTGTCAGT
ACTTTGTTAGAGTGTGTGGAAGTGGGGACGACTCTGATTCTGGTTCCTTA
TGTGGTTATTGTCTGAAACGTTACAGCATTTTGTGAAGTAGGCTTTTGTG
CAAGATTTGATCTCTTTCTCTCATTGTAGAGCTTTAGAGCATTTTTTAGT
ATATGTTTAATCTTTTGATTTTCTAATGTCATTTGCATTCATATTTCACA
TCTTCTTCGTTGGTCTTAAACCAGTTTTCAGATGCTTATCCATTGTCCAC
TTTTCTATGTATCTGTTTCTTCTGTTTGTTTTCAGTATTGTCTTTTATTC
TTTAATCAGTTTTATTTGGACCACAACACCA
SEQ ID NO. 4
AAAGTAATAAAATGGTTCCAAATTTCAATACATCAATGGTTTTCTAATGC
TGACTTGCTTTTCCTCTTTTACTTAAACGATAAGGAAACGAGAGAGGTTG
AAGAAACAAAGAAACTTCCTAAGCATTTACAAATATATCTTCAAAAAATC
TAGTATTTACCAAGTAAAATCATACAAAATAAGAAAAAAAAAGAAGAATC
TATTTCCCAATTTGGTTTTCACCGGAAAAAAAAAACTGTCTTAACAAAAT
CTTAATGCACCAAATCATATAAGAACTTTATTTGACCATTGTCATTAGTT
TTTTTCCTTATGGTAATGGATCTTCAAATAATTTAAATTAAATTAAAAAA
AAAGAAAAAAGAAACAAAAACGCGGAAAGGAGAGCCCAAATCGTCTCCGT
CTGTCATTGTCGCAGTCTCTAAAAGAGAAAAACAAATCGCAACGCTTCAA
TATCTCTCTCTCTCTCCCTATCGTTTGAATCAGTCTCCCAAGATCCACCA
CCACACATGCTACTATGAGCCTCCGCCACCGCACCGTCCCCTCACAACCC
GGACGGCCCCCGGCGGCGGGCGCAATCGACGAGGAGCCCTACAACATCAT
CCCCGTCAACAACCTCCTCGCCGACCACCCCTCCCTCCGCTACCCCGAGG
TCCGCGCCGCCGCCGCCGCCCTCAAAACCGTCGGCGACCTCCGCCGCCCC
ACCTACGTCCAATGGCGCCCCCACTACGACCTCCTCGACTGGCTCGCCCT
CTTCTTCGGCTTCCAGAAGGACAACGTCCGCAACCAGCGCGAGCACCTCG
TCCTCCACCTCGCCAACGCCCAGATGCGCCTCTCCCCGCCGCCGGACAAC
ATCGATTCCCTCGATCCCGCCGTCGTCCGCCGCTTCCGCCGCAAGCTCCT
CGGTAACTACTCCAGCTGGTGCTCCTACCTCGGGAGGAAGTCCAACATCT
GGATCTCGGATCGGACCCCCGATTCGCGGCGGGAGCTTCTCTACGTCGGC
CTCTACCTCCTCGTGTGGGGCGAGGCGGCGAATCTTAGGTTTATGCCTGA
GTGTATCTGTTACATCTTCCACAATATGGCCTCGGAGCTTAACAAGATCT
TAGAGGATTGCCTCGACGAGAGCACGGGGCAGCCGTATTCTCCTAAGATC
ACGGGGGAGAATAGTTTCCTAAACGGCGTCGTTAAGTCAGAACAAAAGGA
AGATTCCATCTAATGGATTATAATCCCACATGCCCATTTTTAAAACTAGA
CAAAATAACTTATAATAGTCCCATAAGAGCATTAAGACAAACTGAACCAA
AAAGAATCTTAAATGCTGGTGAATCTAGTCTCGAATGCTGCTGAACACTC
CTGATATTAGAATTTGGCAGAAAATATTCATACTAATAACAGTAAATACA
TTTCAAAACAAGAAAATAGTAGTAGTAAATAAAAACAATTAATATGTTAT
TATTGTTTCTCTTAGCTGTTACCATTACATTAACAATTTACATTCAATCC
TTTTAATGAAAAATATTATAAAATATTAAAAGAAGTTATTAGAAAAGGAG
AGGGATATGGGGGGGGGGAGAGCATGGAGGGGTCCCATGTGTGTTTCGAA
AAGCAGCAAACATGACTTTGTAAAGTCTTGTTTTAGCTAGAGTTCCAAAA
AAAAAAGAGTTCAAAAAAAAAGAAAAAAAAAAGTCTTGTTTTAGCGTCCA
AAGCGGTCTCACTCGCCGTTCAGCTGTTAAATTTTCTTGAAATAAATCGC
TCCACTTGTTTTTTATTTTTGTTTCTTTCAACCCACTTGTATTAATAACA
ATAATGATATTGCTAATCTTGAACAAATCGTGGATTGTCTTTTCTTCCAG
TGATATCATCCTTAGTCAAAACATCTCAAATTAAATAAATATATAGTTGT
GAATTAAAAAAAAAGGTATAGAGTTTATAAAATTAGCATTTGCGAATGTT
TTTAAAAGTTTGGCGTTTGTATAGATCACTGTCAGTGTCATGAATCATTC
GGTGACAGCAAATTTGTATATGCCAAAAAAGAGAGTTGATACTTGCCATT
CTCGATATTTTGAATTTTTTTTCGTCGACATACAAACTCAACATTTTCGA
AATCTTAGATTAAAAACAAAATGGACAAAACGATACTTATTAATGAATTA
TGTACTAGCGTCTAAACATTAGTAACATAGATATAATTATAAGAACAAGA
AACATATATGCACAAAGCCTCAAACCAAATTTAAATAGTGTTACGAAACA
AGGGATGTACTTGTTATGAAAAATTAATGTATAACCATTCTTAGCATTTT
TTACCTAACCCTTAAACTTTATAAAGAAAAATATGTAACATTTCGGTGTG
TGGTATATAAAAGTATTTTGAAAATCGATTTGATTATTTCTATTATTAAA
ATGTACTTACTTTTTTTATCACACATTTATTTACAATTTCAGAGTTAAAC
ATGATTAAGACAAAGTAGTGAAAATATAGATGATCTACCAGAAAATCATT
TGCTTTCTGAGTGAGATCAAAATAAAGGCAAAAATAATGTGATGATTGTA
TATTAAACAATTATTTTGAATTTGAAAAGAATAACTCATCGAATGAAGCC
TATGAACCGAGTAAGCGGTGAAGCGGTTGGACCGTCACAAAAATTATGAC
CAACAAGAAAATAAAATTGATATTAGTGAATGATATATTTTGGAAAGTAA
TAAATTGGTTCCAGATTTCAATACATCAATGTTTTTCTAATGCTGACTTG
CTTTTCCTCTTTTTCTTACATGATAAGGAAACGAGAGAGGTCGAAGAAAC
AAAGAAACTTCCTAAGCATTTAGCAACGCATCTTCAAAAAATCTAGTATT
TACCAAGTAAAATCATACAAAATAAGGAAAAAAAGAAGAATATATTTCCC
AATTTGGTTTTCACCGGAAAAAAAAAACTGTCTTAACAAAATCTTAATGC
ACCAAATCATATAAGAACTTTATTTGACCATTGTCATTAGTTTTTTCCTT
ATGGTAATGGATCTTCAAACAATTTAAATTAAATTAAAAAAAAAAAGAAA
AGAGAAACAAAAACGCGGAAAGGAGAGCCCAAATCGTCTCCGTCTGTCAT
TGTCGCAGTCTCTAAAAGAGAAAAACAAATCGCAACGCTTCAATATCTCT
CTCTCTCTCTCTCCCTATCGTTTGAATCAGTCTCTCAAGATCCACCACCA
CACATGCTACTATGAGCCTCCGCCACCGCACCGTCCCCTCTCAACCCGGA
CGGCCCCCGGCGGCGGGCGCAATCGAGGACGAGCCCTACAACATCATCCC
CGTCAACAACCTCCTCGCCGACCACCCCTCCCTCCGCTACCCCGAGGTCC
GCGCCGCCGCCGCCGCCCTCAAAACCGTCGGCGACCTCCGCCGCCCCACC
TACGTCCAATGGCGCCCCCACTACGACCTCCTCGACTGGCTCGCCCTCTT
CTTCGGCTTCCAGAAGGACAACGTCCGCAACCAGCGCGAGCACCTCGTCC
TCCACCTCGCCAACGCCCAGATGCGCCTCTCCCCGCCGCCGGACAACATC
GATTCCCTCGATCCCGCCGTCGTCCGCCGCTTCCGCCGCAAGCTCCTCGG
TAACTACTCCAGCTGGTGCTCCTACCTCGGGAGGAAGTCCAACATCTGGA
TCTCGGATCGGACCCCCGATTCGCGGCGGGAGCTTCTCTACGTCGGCCTC
TACCTCCTCGTGTGGGGCGAGGCGGCGAATCTTAGGTTTATGCCTGAGTG
TATCTGTTACATCTTCCACAATATGGCCTCGGAGCTTAACAAGATCTTAG
AGGATTGCCTCGACGAGAGCACGGGGCAGCCGTATTCTCCTAAGATCACG
GGGGAGAATAGTTTCCTAAACGGCGTCGTTAAGCCTATCTACGACACGAT
CAGAGCTGAGATTAATGAGAGCAAGAACGGGACGGAGCCGCATTGTAAGT
GGAGGAACTACGATGATATTAATGAGTACTTCTGGACGGATAGGTGTTTT
AGTAAATTGAAATGGCCGATTGATTTGGGGAGCAGTTTCTTCAAGAACAG
CAGAGGTAGCGGAGTTGGGAAGACAGGTTTTGTGGAGAGGAGGACGTTTT
TCTACCTCTACAGGAGCTTTGATAGGCTTTGGGTGATGCTTGCTTTGTTT
CTTCAAGCTGCTATTATAGTTGCTTGGGAGGAGAAGCCGGGTGGAGGGTC
GGTGACGAGTCAGCTCTGGAATGCGTTGAAGTCGACGGATGTTCAGGTGA
GGCTTTTGACTGTTTTCTTGACGTGGAGTGGGATGAGGTTGTTGCAGGCT
GTGCTGGACGCTGGCTCGCAACGGTCGCTTATTTCTAGAGAGACCAAACG
GCTGTTTTTCAGAATGTTGATGAAGGTTGTGGCTGCTACGGTTTGGATAA
TAGCGTTTATTGTTCTCTACACGAACATCTGGAAGCAGAGGAAGCAAGAT
AGGCAGTGGTCCAGAGCCGCGAATGATAAGATCTATCAGTTCCTTTACGC
TGTGGTGGCTTTCTTGGTCCCTGAGATCCTGGCTTTGGCTCTGTTTATAG
TCCCGTGGATAAGGAACTTTCTGGAAGAGACCAATTGGAAGATATTCTTT
GCTTTGACTTGGTGGTTCCAGGGGAAAAGCTTTGTGGGTCGAGGTTTGAG
AGAGGGGTTGGTGGACAACATCAAGTACTCGACTTTCTGGATCTTTGTCC
TCGCAACGAAGTTCACGTTCAGCTACTTCCTTCAGGTTAAGCCAATGATT
AAACCCTCGAAGCTGCTATGGAATTTGAAGGAGGTGGATTATGAGTGGCA
TCAGTTCTTTGGCGAGAGCAATAGGTTTTCTGTCTTGTTATTGTGGCTGC
CAGTGGTGTTGATATACCTGATGGATATCCAAATTTGGTACGCGATCTAT
TCTTCGATTGTCGGTGCTGTTGTTGGGCTGTTTGATCATCTGGGGGAGAT
CAGGGACATGGGACAGCTTAGGCTGAGGTTCCAGTTCTTTGCTAGCGCTA
TTCAGTTCAACCTAATGCCTGAGGAACAACTCCTGAATGCTAGAGGATTT
GGTAACAAGCTTAAGGACGCCATTCATAGGTAAGTCTATTGAAGCATGTT
ACTGATATTCCTCCTATATAATTTACTATACAGAGTTTGTCTTTACAGTA
CAAGCTATAGGATTTTAGTTTTGTTAAAGCAGATTCTCCGCAATGAACTA
GACTTTTTCACATTGTAATAACATTGAGTTTGTGTACTTTATGCAGATTG
AAGCTGAGGTATGGATTGGGGCGGCCGTTTAAGAAACTCGAGTCCAATCA
GGTTGAGGCTAACAAGTTTGCGCTGATCTGGAATGAGATAATCTTAGCTT
TCAGAGAGGAGGATATAGTCTCTGATCGAGAAGTAGAGCTGCTGGAGCTG
CCAAAGAATTCCTGGAATGTAACAGTTATCCGCTGGCCGTGTTTCCTGTT
GTGCAATGAGCTTTTGCTTGCACTGAGCCAGGCGAAAGAGCTGGTTGACG
CACCTGATAAATGGCTGTGGCACAAGATATGCAAGAATGAGTACAGGCGG
TGTGCTGTGGTTGAGGCATATGAAAGCATCAAACATCTGTTGCTTTCAAT
CATCAAAATTGACACTGAAGAACATAAAATTGTTACAATTTTCTTTCAGA
TGATTGAGGTCTCTATTCAGGGTGAGCAGTTCACCAAGACCTTCAAAGTG
GACCTTTTGCCAAAGATTTATGAGACACTACAGAAGTTGGTTGGTCTGTT
GAATGATGAGAAAGTGGATGTTGGGCGAGTGGTGAATGGTCTGCAATCTA
TTTATGAGATTGCAACACGACAGTTCTTCATAGAAAAGAAGACGACTGAA
CAGCTATCTACTGAGGGGTTAACTCCTCATGATCCAGCCTCAAAGTTACT
GTTTCAGAATGCTGTTAGGCTTCCCGATGCAAGCAATGAAGACTTCTTCC
GGCAGGTTAGGCGGTTACACACAATTCTCACTTCTAGGGACTCTATGCAC
AGCGTCCCTGTGAATCTAGAGGCGAGACGGCGGATTGCCTTCTTCAGCAA
TTCGCTCTTCATGAACTTGCCTCATGCACCTCAGGTGGAGAAAATGTTGG
CGTTCAGTGTTATGACTCCATACTACAGCGAGGAAGTTGTATACAGCAAA
GAACAGCTCCGAAATGAGACTGAGGATGGAATTTCAACCTTGTATTACCT
GCAGACGATTTATGCCGACGAATGGAAAAATTTTAAGGAACGGATGCGTA
GGGAAGGTATAAAGACAGATGTTGAGTTGTGGACAACCAAGCTGAGAGAG
CTCAGGCTTTGGGCTTCCTACAGAGGTCAGACTTTGGCACGTACAGTTCG
AGGAATGATGTACTATTACAGGGCTCTTAAGATGCTTGCTTTTCTTGACT
CTGCGTCTGAAATGGACATTCGGGAGGATGCTCAGGAGCTTGGTTCAATG
AGGAGTTCGCAGGGAAATCGATTGGATGGGGTTGACGATGTAAATGACGG
ATCTTCTCTAAGCAGAGCAACTAGCTCTGTGAGCATGCTGTATAAAGGCC
ATGAGCATGGGACTGCATTGATGAAATTCACATATGTCGTGGCGTGCCAG
ATCTATGGGTCTCAAAAAGCGAAGAAGGAGCCTCAGGCAGAGGAAATTCT
GTATCTTATGAAGCAAAACGAAGCCCTCCGTATTGCATATGTGGATGAGG
TACATGCGGGCAGGGGAGAGACTGAGTATTACTCCGTTCTGGTGAAATAC
GATCATACGTTGGAGAGGGAAGTGGAGATATTCCGTGTGAAGCTACCTGG
TCCGGTGAAGCTGGGTGAGGGAAAGCCAGAGAACCAGAATCATGCAATGA
TCTTTACCCGTGGTGATGCTGTTCAGACCATAGATATGAACCAGGATAAT
TATTTTGAGGAGGCTCTCAAGATGAGAAATTTGCTCCAGGAGTTTAGGCA
TTATCATGGGATCAGAAAACCAACTATTCTTGGTGTCCGGGAGCACATCT
TCACGGGATCTGTCTCGTCTCTGGCATGGTTCATGTCTGCTCAGGAGACT
AGTTTCGTCACTCTTGGTCAGCGTGTTCTCGCCAATCCGCTGAAGGTCAG
AATGCATTATGGTCATCCTGATGTTTTTGACAGATTCTGGTTCTTGAGTC
GAGGTGGCATCAGCAAAGCTTCTAGAGTTATAAATATCAGTGAGGACATC
TTCGCCGGGTTTAATTGCACATTGCGGGGCGGTAACGTCACCCACCACGA
GTATATTCAGGTAGGGAAATGTTCATCATTTGGATATTCTAACTAATTTA
TACATCGACAACAATACTATAATTCCACTTTTTTTGTTATAACCTTCGTG
TGTGTGCATATGTATTCAGGTTGGGAAGGGTCGAGATGTTGGATTGAATC
AAATATCAATGTTTGAGGCTAAGGTAGCCAGTGGGAATGGAGAGCAGGTT
CTTAGCCGAGATGTGTACAGGCTGGGTCATAGGCTCGATTTCTTCAGAAT
GTTATCATTTTTCTACACAACGGTGGGGTTTTTCTTCAACACGATGATGG
TCATTCTCACTGTCTACGCTTTCCTCTGGGGTCGGGTTTATCTTGCTCTG
AGCGGTGTTGAGAAGTCCGCTCTAGCAGACAGCACAGACACCAACGCAGC
GCTTGCTGTGATATTGAACCAGCAATTCATCATTCAGCTTGGTCTCTTCA
CAGCTCTGCCAATGATTGTGGAATGGTCTCTCGAGGAGGGTTTCCTTCTA
GCGATATGGAATTTCATTCGGATGCAGATTCAGCTTTCATCTGTCTTCTA
CACATTCTCAATGGGGACCAGAGCTCACTATTTTGGCCGAACCATTCTCC
ATGGTGGAGCAAAGTACAGAGCCACTGGGCGTGGATTTGTTGTCGAGCAC
AAGAGCTTCACTGAGAATTACCGTCTATACGCACGCAGTCACTTTGTGAA
GGCCATCGAGCTTGGGCTGATCCTCATAGTCTACGCTACGCACAGTCCCA
TCGCCAAAGACTCATTGATCTATATAGCTATGACTCTCACCAGCTGGTTC
CTCGTGATTTCATGGATACTGGCCCCTTTTGTGTTCAACCCGTCAGGATT
CGACTGGCTTAAGACGGTCTATGACTTCGAAGGCTTCATGAACTGGATCT
GGTATCAAGGCAGAATCTCAACGAAGTCCGAACAGAGCTGGGAGATATGG
TGGTATGAGGAACAGGACCACCTGAGAACCACCGGTATACCAGGAAGAAT
CGTGGAGATAATCTTGGACCTTCGGTTTTTCTTCTTCCAGTACGGGATTG
TATACCAGCTCAAAATCGCAAACGGATCAACCAGCATTCTCGTCTACTTA
CTCTCATGGATATACATCTTCGCAGTGTTTGTGTTCTTCCTAGTAATCCA
ATACGCCCGTGACAAGTACTCAGCGAGAAACCACATACGGTACAGGCTCG
TCCAGTTCCTCCTGATCGTGTTTGGTACACTGGTGATTGTTGCTCTCCTC
GAGTTCACGCATTTCAGCTTCGTGGATATCTTCACGAGTCTTCTTGCGTT
CGTCCCAACCGGCTGGGGAATCTTGCTGATCGCACAGGCTTTGAGGCCTG
CGCTGCAGAAGATCGGGCTTATCTGGAACGCGGTTGTCTCCCTTGCTCGG
TTGTATGACATACTGTTCGGGATAGTCATCATGGTTCCCGTAGCGTTCAT
GTCGTGGATGCCTGGGTTTCAATCGATGCAAACGAGGATCTTATTCAATG
AAGCTTTTAGCAGAGGGCTTCGTATCATGCAGATTGTCACTGGGAAGAAA
TCAAAAGGCGATGTCGAAGTTGAAAAAAGAAGGTAAAGCTTCTTATTTAC
CCAAACATCTTTTATGTTCTGTTTGTTTGGAATCTTAAATTACAACAACA
CTAATGCAAAGCTTTTATAACTTGTGTAGGTCTTGAGGTATATGGTAATT
TAAAAGTTGCTGGTTTGCGGCGATGAGGTTAGTTTTGTATTCTTATAAGT
TATGCTTCTTGTCTGAATAAGAACTCAGACACCCGTGTTTTGTCTTCTTC
TTATTTTAAACCAGGTTTTTGGAGAGCTTTGGTTGAGGAAGCTATTTGAT
TAGTTCATCGGTTAGTGGGAGAAACATATATATACATTTGTCAGTACTTT
GTTAGAGTGTGTGGAAATGGGGACGACTCTGATTCTGATTCCTTATGTGG
TTATTGTCTGAAACGTTACAGCATTTTGTGAAGTAGGCTTTTGTGCAAGA
TTTGATATCTTTCTCTCATTGTAGAGCTTTAGAGCATTTTTTAGTATATG
TTTAATCTTTGATTTTTCTAATGTCATTTGCATTCATATTTCAC
SEQ ID NO. 5
GTTTCTTTTTTTTTTTGGTCGAAATTATATTATTGTTTCTCTGAGCTGTT
ACCATTACATTAACAATTTACATTCTATCCTTTTGATGAAAAATATTATA
AAATGTTAAAAGAAGTTATTAGAAAAGGAGAGGAAAACAGGGGGAGAGCA
TGGAGGGGTCCCATGTGTGTTTCGGAAAGCAGCAAACATGACTTTGTAAA
GTCTTGTTTTAGCGTCCAAAGCGGTCTCACTCGCCGTTCAGCGAGCTTTA
GCTGTTAAATTTTCTTGAAATAAATCGCTCCACTTGTTAAAAAAAAAAAT
GATAACTGTGTCCATTTCGACTAATATCTATTTTGTGTCATTTTTGCATA
TAATATTTTTTAATATTTTTGAAAATAAATTTAATAAATAGTTTTATAAA
CAAAAAAAATTGAAAAATAGTAACTATTGATAAAATACCTATATGAACTT
AGTAGTATTTTTTTTCAGTTCTAAAAATGTTTAAATCATAATTTTCAGAT
TATGTTATAAATAAAGCAGAAATGACATTCTACGAAGTAGAAAACGAAAT
CCATTTTTTTTCATTGAATCTACAATGTTTAGAATACGAAATCTTCGTAA
ATAAGAGTATTCTAAAAATATTTAGAATACACATTCCGCGCTTAACCTAC
CGATCTAAAATCTTTAGAAATCAAAATCTACAGATTAATATAAATCTAAA
ACACGTTGAAACCGACTTCTACAGATTTACTGTAAATCTAAAACATGTAG
AAATCAAAATTCTACACATTACTATAATTCTAAAAACCTATAGAAACCGA
TTTCTACATATTATCTGTATTCTACGGATAAGAAATCAGTTCTTAAAAAT
ATGGAAACAAAATATTTGAGAATATTCACTTTCATATTTTGAAAAAAATC
GATTTAAAAAAAACAAAAAAAACAAAAAACCTTCTTTTCACGCCGTCTTC
TATATTCCATTGATTGTTCACAAAATTTTCACATTATTTCCACCATATCA
GTGTGAAATTGAGTGAGTTAGGGTTTATGAACTTGAGACTTTGATTTTCT
CCCTTTTTGTTCGTGAAGGAGATGAGAAATTATGAGTTTCATCTCAAAGA
AATCGAGTTTAAAGAGTTGAAATCGTGTTTGGTCGGTTCAAATCAAGTGG
GAATTAAACCGGATATAACCGAAGAAAACCAGGAAATCGATTTGGAATAC
TTACCTAGGCACAAGATCGATCGGTCTATAATTAGAGATCGGTTGATCTG
TATGAAATCGACTTTTGCCGATCGAGTGAAATGGACCAGGTCGATCAGCC
GCGTTTTTTTGTTCTGCATAATGATCAGAATCGGTCAATCCGTCCGACCT
TGTAATTTCGGATCGATCGATCCCGCTTGATCGAACCTATTATTTATTAT
ATTTAAGAATTACAATTTTAAGGATTTTATTGCCATTTTGAAAATAATTA
GTCTAATTGAACATAAAATATATAAGATTAGTCTAAAATGACATAGTTAT
CATTTTTTGTTCTAAAAAAACAATTTCCCTTTTTTATTTTTGTTTCTTTC
AACACACTTGTATTAATAACAATAATGATATTGCTAATCTTGAACAAATC
GTGGATTGTCTTTTCTTCCGGTGATATCATCCTTAGTCAAAACATCTCAA
ATTAAATAAGTATAGAGTTGTGAATGTTTTTAAAAGTTTGGCGTTTGTAT
AGATCACTGTCAGTGTCATGAATCATTAGGTGACTGCAAATTTTTATATG
ACAAAAAAGAGAGTTGATACTTGCCATTCTCGAGATTTTGAAGTTTTTTT
CGTCGTCAACATACAAACTCAACATTTTCGAAATCTTGGATTAAAAATAA
AAATATGGACAAAACGATACTTATTAATTAATTATGTACTAGCGTCTTAA
CATTATTAATATAGATATAATTATAAGAACAAGAAACATAAACATATATG
CACAAAGCCTCAAAGCAAATTTAAATAATGTTACAAAACAAGGGATGTAC
TTGTTATGAAAAATTAATATATAACCATTCTTAACATATTTTTACGTAAA
CCTTAAACTTTATAAAGAAAAATATGTAACATTCCGGTGCGTGGTATATA
GAAATATTTTTAAAAATTGATTTGACTATTTCCATTAGCAAAATGCATTT
ATTTTTTCGATCATACATTTATTTAAAATTTCAGGGTTAAACGTGATTAA
AATGGAGTAATAAGAATATGGATGATCTACCAGAAAATCATTTACGTTTT
AAGTGAGACCAAAATACGGATAAAAATCATGTGATGATTGTATGGTTATT
AAACAATTATTTTGAATTTGAAAATAATTAACTCATCAAATTAAACCTAA
GGACCGAGTAAGCGGACGCAGATAGCCGTAAGCGGTTGGGCCGTCACAAA
ATTTATGACCAACAAGAAAATGAAACTGATTTTAGTGGATGATATATTTT
AGAAAGTAATAAAATGGTTCCAGATTTCAATACTTCAATGGTTTTTTTAT
GCTGACTTTCTTTTCCTCTTTTACTTACATGATAAGGAAACGAGAGAGGT
TGAAGAAACAAAGAAAATTCCTAAGGATTTAGAAATGTATCTTCAAAAAA
TCTAAGTATTTACCAAGTAAAATCATACAAAATAAGGAAAAAAAAAGAAT
TTATTTCCCAATTTGGTTTTTACCGGAAAAAAAATTGTTTTAATAAAATC
TTATGCACCAAATCATATAAGAACGAACCATAACAAACTTTATTTGACCA
TTGTCATTAGTTTATTTTATTTATGGTAATGGATCTTCAAATAATTTGAA
TTAAATTAAAAAAAAGAATAAAAAAAGAGAAACAAAAACGCGGAAAGGAG
AGCCCAAATCGTCTCCGTCTGTCATTGTCGCAGTCTCTCACAGCTAAATA
AAAGAGAAAAACAAATCGCAACGCTTCAATATCTCTCTCCCAATCGTTTG
AATTAGTCTTCCTCATCACAATACCCCTAAGATCCACCTCACATGCTACT
ATGAGCCTCCGCCACCGCACCACCGTCCCCTCTCAACCCGGACGGCCCCC
GGCGGCGGGCGCAATCGACGAGGAGCCCTACAACATCATCCCCGTCAACA
ACCTCCTCGCCGACCACCCCTCCCTCCGCTACCCCGAGGTCCGCGCCGCC
GCCGCCGCCCTCAAAACCGTCGGAGACCTCCGCCGCCCCACCTACGTCCA
ATGGCGCCCCCACTACGACCTCCTCGACTGGCTCGCCCTCTTCTTCGGCT
TCCAGAAGGACAACGTCCGCAACCAGCGCGAGCACCTCGTCCTACACCTC
GCCAACGCCCAGATGCGCCTCACGCCGCCGCCGGATAACATCGATTCCCT
CGATCCCGCCGTCGTCCGCCGTTTCCGCCGCAAGCTCCTCGGTAACTACT
CGAGCTGGTGCTCGTACCTCGGGAGGAAGTCGAACATCTGGATCTCGGAT
CGGAACCCCGATTCGAGGCGGGAGCTTCTCTACGTCGGCCTCTACCTCCT
CGTGTGGGGGGAGGCGGCGAATCTTAGGTTTATGCCGGAGTGTATCTGTT
ACATCTTCCACAATATGGCCTCGGAGCTTAACAAGATCCTCGAGGATTGC
CTCGACGAGAGCACGGGGCAGCCGTATTCTCCTAGAATCACGGGGGAGAA
TAGTTTCCTAAACGGCGTCGTTAAACCTATTTACGAGACGATCAAAGCTG
AGATTAACGAGAGCAAGAACGGGACGGAGCCGCATTGTAAGTGGAGGAAC
TATGATGATATTAACGAGTACTTTTGGACGGATAGGTGTTTCAGTAAATT
GAAATGGCCGATTGATTTGGGGAGCAGTTTCTTCAAGAGTAGTAGAGGGA
GCGGCGTTGGGAAGACAGGTTTTGTGGAGCGGAGGACGTTTTTTTACCTC
TACAGGAGCTTTGATAGGCTTTGGGTGATGCTTGCTTTGTTTCTTCAAGC
TGCTATTATAGTTGCTTGGGAGGAGAAGCCGGGTGGAGGGTCGGTGAGGA
GTCAGCTCTGGAATGCGTTGAAGTCGACGGATGTTCAGGTGAGGCTTTTG
ACTGTGTTTTTGACGTGGAGTGGGATGAGATTACTGCAGGCTGTGCTGGA
CGCTGGCTCGCAACGGCCGCTTATTTCTAGAGAGACCAAACGGCTGTTTT
TCAGAATGCTGATGAAGGTTGTAGCTGCTACGGTTTGGATCATTGCTTTT
ATTGTTCTCTACACGAACATCTGGAAGCAGAGGAAGCAAGACAGGCAGTG
GTCCAGAGCCGCGAATGACAAGATCTACCAGTTCCTTTACGCTGTGGTGG
CTTTCTTGGTCCCTGAGATCCTGGCTTTGGCTCTGTTTATAGTCCCGTGG
ATAAGGAACTTTCTGGAAGAGACCAATTGGAAGATATTCTTTGCTTTGAC
TTGGTGGTTCCAGGGTAAAAGCTTTGTGGGTCGAGGTTTGAGAGAGGGGT
TGGTGGACAACATCAAGTACTCGACTTTCTGGATCTTTGTCCTAGCAACG
AAGTTCACGTTCAGCTACTTCCTGCAGGTTAAGCCAATGATTAAACCCTC
GAAGCTGCTATGGAATTTGAAGGAGGTGGATTATGAGTGGCATCAGTTCT
TTGGCAAGAGCAATAGGTTTTCTGTCTTGTTATTGTGGCTGCCAGTGGTG
TTGATATACCTGATGGATATCCAAATTTGGTACGCGATCTATTCTTCGAT
TGTTGGTGCTGTTGTTGGGCTGTTTGATCATCTGGGGGAGATCAGGGACA
TGGGACAGCTTAGGCTGAGGTTTCAGTTCTTTGCTAGCGCTATTCAGTTC
AACCTAATGCCTGAGGAACAACTCCTGAATGCTAGAGGATTTGGTAACAA
GCTTAAGGACGCCATTCATAGGTAAGTCTATTGAAGCATGTTACTGATAT
TTCTATATAATTTATTATACAGAGTTTGTCTTTACAGTACAAGCTATAGG
ATTTTAGTTTTGTTAAAGAAGATTTCTCCGCAATGAACTCGACTTTTCAC
ATTGTAATAACATTGAGTTTGTGTACTTTATGCAGATTGAAGCTGAGGTA
TGGATTGGGGCGGCCGTTTAAGAAACTCGAGTCCAATCAGGTTGAGGCTA
ACAAGTTTGCGCTGATCTGGAATGAGATAATCTTAGCTTTCAGAGAGGAG
GATATAGTCTCTGATCGAGAAGTAGAGCTGCTGGAGCTGCCAAAGAATTC
CTGGAATGTGACAGTTATCCGCTGGCCGTGTTTCCTGTTGTGCAACGAGC
TTTTGCTTGCACTGAGCCAGGCGAAAGAGCTGGTTGACGCACCTGATAAA
TGGCTGTGGCACAAGATATGCAAGAATGAGTACAGGCGGTGTGCTGTGGT
TGAGGCATATGAAAGCATCAAACATCTGTTGCTCTCAATCATCAAAATTG
ACACCGAAGAACATAAAATTGTTACAATTTTCTTTCAGATGATTGAGGTG
TCTATTCAGGGTGAGCAGTTCACCAAGACCTTCAAAGTGGACCTATTGCC
AAAGATTTATGAGACGCTACAGAAGTTGGTTGGGCTGTTGAATGATGAGA
AAGTGGATGTTGGGCGAGTGGTGAATGGTCTGCAGTCTATTTATGAGATT
GCAACACGACAGTTCTTCATAGAAAAGAAGACGACTGAACAGCTATCTAC
CGAGGGGTTAACTCCTCATGATCCAGCCTCAAAGTTACTGTTTCAGAATG
CTGTTAGGCTTCCCGATGCAAGCAATGAAGACTTCTTCCGGCAGGTTAGG
CGGTTACACACAATTCTCACTTCTAGGGACTCTATGCACAGCGTCCCTGT
GAATCTAGAGGCGAGACGGCGGATTGCCTTCTTCAGCAATTCGCTCTTCA
TGAACTTGCCTCATGCACCTCAGGTGGAGAAAATGTTGGCGTTCAGTGTT
ATGACTCCATACTACAGCGAGGAAGTTGTATACAGTAAAGAACAGCTCCG
AAATGAGACTGAGGATGGGATTTCAACCTTGTATTACCTGCAGACGATTT
ATGCCGACGAATGGAAAAATTTTAAGGAACGGATGCGTAGGGAAGGTATA
AAGACAGATGTTGAGTTGTGGACAACCAAGCTGAGAGAGCTCAGGCTTTG
GGCTTCCTACAGAGGTCAGACTTTGGCACGTACAGTTCGAGGAATGATGT
ACTATTACAGGGCTCTTAAGATGCTTGCTTTTCTCGACTCTGCGTCTGAA
ATGGACATTCGGGAGGATGCTCAGGAGCTTGGTTCAATGAGGAGTTCGCA
GGGAAATCGATTGGATGGTGTTGACGATGTAAATGACCGATCTTCTCTAA
GCAGAGCAACTAGCTCTGTGAGCATGCTGTATAAAGGCCATGAGTATGGG
ACTGCATTGATGAAATTCACATATGTCGTGGCGTGCCAAATCTATGGGTC
TCAAAAAGCGAAGAAAGAGCCTCAGGCAGAGGAAATTCTGTATCTTATGA
AGCAAAACGAAGCCCTTCGTATTGCATATGTGGATGAGGTACATGCGGGC
AGGGGAGAGACTGAGTATTACTCCGTTCTGGTGAAATACGATCACACGTT
GGAGAGGGAAGTGGAGATATTCCGTGTGAAGCTACCTGGTCCGGTGAAGC
TGGGTGAGGGAAAGCCAGAGAACCAGAATCATGCAATGATCTTTACCCGT
GGTGATGCTGTTCAGACCATAGATATGAACCAGGATAATTATTTTGAGGA
GGCTCTCAAGATGAGAAATTTGCTCCAGGAGTTTAGGCATTATCATGGGA
TCAGAAAACCAACTATTCTTGGTGTCAGAGAGCACATCTTCACGGGTTCT
GTCTCGTCTCTGGCTTGGTTCATGTCTGCTCAGGAGACAAGTTTCGTCAC
TCTTGGTCAGCGTGTTCTAGCCAACCCGCTGAAGGTCAGAATGCATTATG
GTCACCCTGATGTATTTGACAGATTCTGGTTCTTGAGTCGAGGTGGCATC
AGCAAAGCTTCTAGAGTTATAAATATCAGTGAGGACATCTTCGCCGGGTT
TAATTGCACATTGCGGGGCGGTAACGTCACCCACCACGAGTATATTCAGG
TAGGGAAATGTTCATCATTTGGATATTCTAACTAATTTATACATCGACAA
CAATACTATAATTCCACTTTTTTGTTATAACCTTTTTGTGTGTGCATATG
TATTCAGGTTGGGAAGGGTCGAGATGTTGGATTGAATCAAATATCAATGT
TTGAGGCTAAGGTAGCCAGTGGGAATGGAGAGCAGGTTCTTAGCCGAGAT
GTGTACAGGTTGGGTCATAGGCTCGATTTCTTCAGAATGTTATCATTTTT
CTACACAACGGTGGGGTTTTTCTTCAACACGATGATGGTCATTCTCACTG
TCTACGCTTTCCTCTGGGGCCGGGTTTATCTTGCTCTGAGCGGTGTTGAG
AAGTCCGCTCTAGCAGACAGCACAGACACCAACGCAGCGCTTGCTGTGAT
ATTGAACCAGCAGTTCATCATTCAGCTTGGTCTCTTCACAGCTCTGCCAA
TGATTGTGGAATGGTCTCTCGAGGAGGGTTTCCTTCTCGCGATATGGAAC
TTCATTCGGATGCAGATTCAGCTTTCTTCTGTCTTCTACACATTCTCAAT
GGGGACCAGAGCTCACTATTTTGGCCGAACCATTCTCCACGGTGGAGCAA
AGTACAGAGCCACTGGACGTGGATTTGTTGTCGAGCACAAGAGTTTCACT
GAAAACTACCGTCTATACGCGCGCAGTCACTTTGTGAAGGCCATCGAGCT
TGGGCTGATCCTCATAGTCTACGCTACGCACAGTCCCATCGCCAAAGACT
CATTGATCTATATAGCCATGACTCTCACCAGCTGGTTCCTCGTGATTTCA
TGGATACTAGCCCCTTTTGTGTTCAACCCGTCAGGTTTCGACTGGCTTAA
GACGGTCTACGACTTCGAAGGCTTCATGAACTGGATCTGGTATCAAGGCA
GAATCTCAACGAAGTCCGAACAGAGCTGGGAGATATGGTGGTATGAGGAA
CAGGACCACCTGAGAACCACCGGTCTACCAGGAAGAATCATGGAGATAAT
CTTGGACCTTCGGTTTTTCTTCTTCCAGTACGGGATTGTATACCAGCTCA
AAATCGCAAACGGATCAACCAGCGTTCTCGTCTACTTACTCTCATGGATA
TACATCTTCGCAGTGTTTGTGTTCTTCCTGGTAATCCAATACGCCCGTGA
CAAGTACTCAGCGAGAAACCACATACGGTACAGGCTCGTTCAGTTCCTCC
TGATCGTGTTTGGTACACTGGTGATTGTTGCTCTCCTGGAGTTCACGCAT
TTCAGCTTCGTGGATATCTTCACGAGTCTTCTTGCGTTCGTCCCAACCGG
CTGGGGAATCTTGCTGATCGCACAGGCTTTGAGGCCTGCGCTGCAGAAGA
TCGGGCTTATCTGGAACGCGGTTATCTCCCTTGCTCGGTTATATGACATA
CTGTTCGGGATAGTCATCATGGTCCCCGTAGCGTTCATGTCGTGGATGCC
TGGGTTTCAGTCGATGCAAACGAGGATCTTATTCAATGAAGCTTTTAGCA
GAGGGCTTCGTATCATGCAGATTGTCACTGGGAAGAAATCAAAAGGCGAT
GTCGAAGTTGAAAAAAGAAGGTAA
SEQ ID NO. 6
CTGTCAGATTTAGTTTCACAAAATCTTTGTAACTTCGCGGTTTTTGATAT
GGAAGTTGCTACAAAAACTGACTTGTGATATTATAATCTTTCGGCAAGAA
AAATATATAATACTAATTTCAGAAAGTATTCTGTTGAATTGTTGATCACC
AAATACACATAAACCGGCAGATGTTGACATGTTTACGTGGGATCACTGGG
ATAGAAGTTTTGCTTCTGTATTAGTATCGCGAACCGGTTTGTAGTAGCGA
AAATAACATTATTATTTTCAGATTTCATCATCGTCAAATTCAACGTTACA
TTTGAACAGGCTTGTAAAAAGTGGACATGTGGCATGAGTAAAAGATAAAG
AAAATTGTGAGCCTTTTTGTTAATACGAAAAAGCCGAAAACGTTATTATT
TCTATCATCGGCAAATTTAATGTTAAATTTGCTCCACATCATTCAGAGGG
TATATGTATATTTTTCTAATGCTTCGGTGAAGCACATGCGACCATTTGTT
GCGTCAGAAGTCAGAACAAAAGGAAGATTCCATCTAATGGATTATAATCC
CACATGCCCATTTTTAAAACTAGACAAAATAACTTATAATAGTCCCATAA
GAGCATTAAGACAAACTGAACCAAAAAGAATCTTAAATGCTGGTGAATCT
AGTCTCGAATGCTGCTGAACACTCCTGATATTAGAATTTGGCAGAAAATA
TTCATACTAATAACAGTAAATACATTTCAAAACAAGAAAATAGTAGTAGT
AAATAAAAACAATTAATATGTTATTATTGTTTCTCTTAGCTGTTACCATT
ACATTAACAATTTACATTCAATCCTTTTAATGAAAAATATTATAAAATAT
TAAAAGAAGTTATTAGAAAAGGAGAGGGATATGGGGGGGGGAGAGCATGG
AGGGGTCCCATGTGTGTTTCGAAAAGCAGCAAACATGACTTTGTAAAGTC
TTGTTTTAGCTAGAGTTCCAAAAAAAAAAGAGTTCAAAAAAAAAGAAAAA
AAAAAGTCTTGTTTTAGCGTCCAAAGCGGTCTCACTCGCCGTTCAGCTGT
TAAATTTTCTTGAAATAAATCGCTCCACTTGTTTTTTATTTTTGTTTCTT
TCAACCCACTTGTATTAATAACAATAATGATATTGCTAATCTTGAACAAA
TCGTGGATTGTCTTTTCTTCCAGTGATATCATCCTTAGTCAAAACATCTC
AAATTAAATAAATATATAGTTGTGAATTAAAAAAAAGGTATAGAGTTTAT
AAAATTAGCATTTGCGAATGTTTTTAAAAGTTTGGCGTTTGTATAGATCA
CTGTCAGTGTCATGAATCATTCGGTGACAGCAAATTTGTATATGCCAAAA
AAGAGAGTTGATACTTGCCATTCTCGATATTTTGAATTTTTTTTCGTCGA
CATACAAACTCAACATTTTCGAAATCTTAGATTAAAAACAAAATGGACAA
AACGATACTTATTAATGAATTATGTACTAGCGTCTGAACATTAGTAACAT
AGATATAATTATAAGAACAAGAAACATATATGCACAAAGCCTCAAACCAA
ATTTAAATAGTGTTACGAAACAAGGGATGTACTTGTTATGAAAAATTAAT
GTATAACCATTCTTAGCATTTTTTACCTAACCCTTAAACTTTATAAAGAA
AAATATGTAACATTTCGGTGTGTGGTATATAAAAGTATTTTGAAAATCGA
TTTGATTATTTCTATTATTAAAATGTACTTACTTTTTTTATCACACATTT
ATTTACAATTTCAGAGTTAAACATGATTAAGACAAAGTAGTGAAAATATA
GATGATCTACCAGAAAATCATTTGCTTTCTGAGTGAGATCAAAATAAAGG
CAAAAATAATGTGATGATTGTATATTAAACAATTATTTTGAATTTGAAAA
GAATAACTCATCGAATGAAGCCTATGAACCGAGTAAGCGGTGAAGCGGTT
GGACCGTCACAAAAATTATGACCAACAAGAAAATAAAATTGATATTAGTG
AAGGGCAATTGTCAATAATAGCACCTTTTGAAGTTTATGTCTCAAAAATA
GCACTAGAAGGAGAAAGTCACAAAAATGACATTCATTAAAGGGTAAAATA
TCTATAATACCCTTGGTTTAAAATTAAATAAACAAACTAAAATAAATAAA
AATAAAAAAATAAAAAAAATAAAAATAAAAAAAATAAATTTTTTTTTATA
GTTTCAGATTATATGTTTTCAGATTCGAAATTTTTATAATTTTTTTTTAA
AAAAAAATTTTAAATCTTTTTTTTATTTTTTTTTCAGATTTTATTTTTAT
AATTTAAAAATACTTTTTGAAACTGTTTTTAAAATTTTTATTTTTTATTT
TATTATTTATTATTTATAAAATTTTAAATCCTAATTCCAAAACCCCACCC
CTTAACTCTAAACCCTAATGTTTGGATTAATTAACCCTAGGGGTATAAGT
GTACATTACCTCTTTAATGAAACCTATTTTTGTGACTTTGAACCTTGAGT
GCTACTTTGGGAACAAAAACTTGGTTTGGTGCTATTCTAGTATTTTTCTC
ATTAGTGAATGATATATTTTAGAAAGTAATAAATTGGTTCCAGATTTCAA
TACATCAATGTTTTTCTAATGCTGACTTGCTTTTCCTCTTTTTCTTACAT
GATAAGGAAACGAGAGAGGTCGAAGAAACAAAGAAACTTCCTAAGCATTT
AGCAACGCATCTTCAAAAAATCTAGTATTTACCAAGTAAAATCATACAAA
ATAAGGAAAAAAAGAAGAATATATTTCCCAATTTGGTTTTCACCGGAAAA
AAAAAACTGTCTTAACAAAATCTTAATGCACCAAATCATATAAGAACTTT
ATTTGACCATTGTCATTAGTTTTTTCCTTATGGTAATGGATCTTCAAATA
ATTTAAATTAAATTAAAAAAAAAAAGAAAAGAGAAACAAAAACGCGGAAA
GGAGAGCCCAAATCGTCTCCGTCTGTCATTGTCGCAGTCTCTAAAAGAGA
AAAACAAATCGCAACGCTTCTCTCTCTCTCCCTATCGTTTGAATCAGTCT
CTCAAGATCCACCACCACCACACATGCTACTATGAGCCTCCGCCACCGCA
CCGTCCCCTCTCAACCCGGACGGCCCCCGGCGGCGGGCGCAATCGAGGAC
GAGCCCTACAACATCATCCCCGTCAACAACCTCCTCGCCGACCACCCCTC
CCTCCGCTACCCCGAGGTCCGCGCCGCCGCCGCCGCCCTCAAAACCGTCG
GCGACCTCCGCCGCCCCACCTACGTCCAATGGCGCCCCCACTACGACCTC
CTCGACTGGCTCGCCCTCTTCTTCGGCTTCCAGAAGGACAACGTCCGCAA
CCAGCGCGAGCACCTCGTCCTCCACCTCGCCAACGCCCAGATGCGCCTCA
CCCCGCCGCCGGACAACATCGATTCCCTCGATCCCGCCGTCGTCCGCCGC
TTCCGCCGCAAGCTCCTCGGTAACTACTCCAGCTGGTGCTCCTACCTCGG
GAGGAAGTCCAACATCTGGATCTCGGATCGGACCCCCGATTCGCGGCGGG
AGCTTCTCTACGTCGGCCTCTACCTCCTCGTGTGGGGCGAGGCGGCGAAT
CTTAGGTTTATGCCTGAGTGTATCTGTTACATCTTCCACAATATGGCCTC
GGAGCTTAACAAGATCCTCGAGGATTGCCTCGACGAGAGCACGGGGCAGC
CGTATTCTCCTAAGATCACGGGGGAGAATAGTTTCCTAAACGGCGTCGTT
AAGCCTATCTACGAGACTATTAAAGCTGAGATTAACGAGAGCAAGAACGG
GACGGAGCCGCATTGTAAGTGGAGGAACTATGATGATATTAATGAGTACT
TTTGGACGGATAGGTGTTTTAGTAAATTGAAATGGCCGATTGATTTGGGG
AGCAGTTTCTTCAAGAACAGCAGAGGTAGCGGCGTTGGGAAGACTGGTTT
TGTGGAGAGGAGGACGTTTTTTTACCTCTACAGGAGCTTTGATAGGCTTT
GGGTGATGCTTGCTTTGTTTCTTCAAGCTGCTATTATAGTTGCTTGGGAG
GAGAAGCCGGGTGGAGGGTCGGTGACGAGTCAGCTCTGGAATGCGTTGAA
GTCGACGGATGTTCAGGTGAGGCTTTTGACTGTGTTCTTGACGTGGAGTG
GGATGAGGTTGTTGCAGGCTGTGTTGGACGCTGGCTCGCAACGGTCGCTT
ATTTCTAGAGAGACCAAACGGCTGTTTTTCAGAATGTTGATGAAGGTTGT
GGCTGCTACGGTTTGGATAGTAGCGTTTATTGTTCTCTACACGAACATCT
GGAAGCAGAGGAAGCAAGATAGGCAGTGGTCCAGAGCCGCGAATGATAAG
ATTTATCAGTTCCTTTACGCTGTGGTGGCTTTCTTGGTCCCTGAGATCCT
GGCTTTGGCTCTGTTTATAGTCCCGTGGATAAGGAACTTTCTGGAAGAGA
CGAATTGGAAGATATTCTTTGCTTTGACTTGGTGGTTCCAGGGGAAAAGC
TTTGTGGGTCGAGGTTTGAGAGAGGGGTTGGTGGACAACATCAAGTACTC
GACTTTCTGGATCTTTGTCCTTGCAACGAAGTTTACGTTCAGCTACTTCC
TGCAGGTTAAGCCAATGATTAAACCCTCGAAGCTGCTATGGAATTTGAAG
GAGGTGGATTATGAGTGGCATCAGTTCTTTGGCGAGAGCAATAGGTTTTC
TGTCTTGTTATTGTGGCTGCCAATGGTGTTGATATACCTGATGGATATCC
AAATTTGGTACGCGATCTATTCTTCGATTGTTGGTGCTGTTGTTGGGCTG
TTTGATCATCTGGGGGAGATCAGGGACATGGGACAGCTTAGGCTGAGGTT
TCAGTTCTTTGCTAGCGCTATTCAGTTCAACCTAATGCCTGAGGAACAAC
TCCTGAATGCTAGAGGATTTGGTAACAAGCTTAAGGACGCCATTCATAGG
TAAGTCTATTGAAGCATGTTACTGATATTTCTATATAATTTACTATATAG
TTTGTCTTTACAGTACAAGCTATGGGATTTTAGTTTTGTTAAAGCAGATT
TCTCCGCAATGAACTCGACTTTTCACATTGTAATAACATTGAGTTTGTGT
ACTTTATGCAGATTGAAGCTGAGGTATGGATTGGGGCGGCCGTTTAAGAA
ACTCGAGTCCAATCAGGTTGAGGCTAACAAGTTTGCGCTGATCTGGAATG
AGATAATCCTAGCTTTCAGAGAGGAGGATATAGTCTCTGATCGAGAAGTA
GAGCTGCTGGAGCTGCCAAAAAATTCCTGGAATGTGACAGTTATCCGCTG
GCCGTGTTTTCTGTTGTGCAACGAGCTTTTGCTTGCACTGAGCCAGGCGA
AAGAGCTGGTTGACGCACCTGATAAATGGCTGTGGCACAAGATATGCAAG
AATGAGTACAGGCGGTGTGCTGTGGTTGAGGCATATGAAAGCATCAAACA
TCTGTTGCTCTCAATCATCAAAATTGACACCGAAGAACATAAAATTGTTA
CAATTTTCTTTCAGATGATTGAGGTGTCTATTCAGGGTGAGCAGTTCACC
AAGACCTTCAAAGTGGACCTTTTGCCAAAGATTTATGAGACACTACAGAA
GTTGGTTGGGCTGTTGAATGATGAGAAAGTGGATGTTGGGCGAGTGGTGA
ATGGTCTGCAGTCTATTTATGAGATTGCAACACGACAGTTCTTCCTAGAA
AAGAAGACGACTGAACAGCTATCTACTGAGGGGTTAACTCCTCATGATCC
AGCCTCAAAGTTACTGTTTCAGAATGCTGTTAGGCTTCCCGATGCAAGCA
ATGAAGACTTCTTCCGGCAGGTTAGGCGGTTACACACAATTCTCACTTCT
AGGGACTCTATGCACAGCGTCCCTGTGAATCTAGAGGCGAGACGGCGGAT
TGCCTTCTTCAGCAATTCGCTCTTCATGAACTTGCCTCATGCACCTCAGG
TGGAGAAAATGTTGGCGTTCAGTGTTATGACTCCATACTACAGCGAGGAA
GTTGTATACAGCAAAGAACAGCTCCGAAATGAGACTGAGGATGGGATTTC
AACCTTGTATTACCTGCAGACGATTTATGCCGACGAATGGAAAAATTTTA
AGGAACGGATGCGGAGGGAAGGTATAAAGACAGATGTTGAGTTGTGGACA
ACCAAGCTGAGAGAGCTCAGGCTTTGGGCTTCCTACAGAGGTCAGACTTT
GGCACGTACAGTTCGAGGAATGATGTACTATTACAGGGCTCTTAAGATGC
TTGCTTTTCTTGACTCTGCGTCTGAAATGGACATTCGGGAGGATGCTCAG
GAGCTTGGTTCAATGAGGAGTTCGCAGGGAAATCGATTGGATGGGGTGGA
CGATGTAAATGACGGATCTTCTCTAAGCAGAGCAACTAGCTCTGTGAGCA
TGCTGTATAAAGGCCATGAGCATGGGACTGCATTGATGAAATTCACATAT
GTCGTGGCGTGCCAGATCTATGGGTCTCAAAAAGCGAAGAAGGAGCCTCA
GGCCGAGGAAATTCTGTATCTTATGAAGCAAAACGAAGCCCTCCGTATTG
CATATGTGGATGAGGTGCATGCGGGCAGGGAAGAGACTGAGTATTACTCC
GTTCTGGTGAAATACGATCACACGTTGGAGAAGGAAGTGGAGATATTCCG
TGTGAAGCTACCTGGTCCGGTGAAGCTGGGTGAGGGAAAGCCAGAGAACC
AGAATCATGCAATGATCTTTACCCGCGGTGATGCTGTTCAGACCATAGAT
ATGAACCAGGATAATTATTTTGAGGAGGCTCTCAAGATGAGAAATTTGCT
CCAGGAGTTTAGGCATTACCATGGGATCAGAAAGCCAACTATTCTTGGTG
TCCGGGAGCACATCTTCACGGGATCTGTCTCGTCTCTGGCTTGGTTCATG
TCTGCTCAGGAGACAAGTTTCGTCACTCTTGGTCAGCGTGTTCTAGCCAA
CCCGCTGAAGGTCAGAATGCATTATGGTCACCCTGATGTATTTGACAGAT
TCTGGTTCTTGAGTCGAGGTGGCATCAGCAAAGCTTCTAGAGTTATAAAT
ATCAGTGAGGACATCTTCGCCGGGTTTAATTGCACATTGCGGGGCGGTAA
CGTCACCCACCACGAGTATATTCAGGTAGGGAAATGTTCATCATTTGGAT
ATTCTAACTAATTTATACATCGACAACAATACTATAATTCCACTTTTTTT
TTGTTATAACCTTTTTGTGTGTGCATATATGTATTCAGGTTGGGAAGGGT
CGAGATGTTGGATTGAATCAAATATCAATGTTTGAGGCTAAGGTAGCCAG
TGGGAATGGAGAGCAGGTTCTTAGCCGAGATGTGTACAGGCTGGGTCATA
GGCTCGATTTCTTCAGAATGTTATCATTTTTCTACACAACGGTGGGGTTT
TTCTTCAACACGATGATGGTCATTCTCACTGTCTACGCTTTCCTCTGGGG
CCGGGTTTATCTTGCTCTGAGCGGTGTTGAGAAGTCCGCTCTAGCAGACA
GCACAGACACCAACGCAGCGCTTGCTGTGATATTGAACCAGCAGTTCATC
ATTCAGCTTGGTCTCTTCACAGCTCTGCCAATGATTGTGGAATGGTCTCT
CGAGGAAGGTTTCCTTCTCGCGATATGGAACTTCATTCGGATGCAGATTC
AGCTTTCCTCTGTCTTCTACACATTCTCAATGGGGACCAGAGCTCACTAT
TTTGGCCGAACCATTCTCCACGGTGGAGCAAAGTACAGAGCCACTGGACG
TGGATTTGTTGTCGAGCACAAGAGTTTCACTGAAAACTACCGTCTATACG
CACGCAGTCACTTTGTGAAGGCCATCGAGCTTGGGCTGATCCTCATAGTC
TACGCTACGCACAGTCCCATCGCCAAAGACTCATTGATCTACATAGCCAT
GACTCTCACCAGCTGGTTCCTCGTGATTTCATGGATACTGGCCCCTTTTG
TGTTCAACCCGTCAGGATTCGACTGGCTTAAGACGGTCTATGACTTCGAA
GGCTTCATGAACTGGATCTGGTATCAAGGCAGAATCTCAACGAAGTCCGA
ACAGAGCTGGGAGATATGGTGGTATGAGGAACAGGACCACCTGAGAACCA
CCGGTATACCAGGAAGAATCATGGAGATAATCTTGGACCTTCGGTTTTTC
TTCTTCCAGTACGGGATTGTATACCAGCTCAAAATCGCAAACGGATCAAC
CAGCATTCTCGTCTACTTACTCTCATGGATATACATCTTTGCAGTGTTTG
TGTTCTTCCTAGTGATCCAATACGCCCGTGACAAGTACTCTGCGAGAAAC
CACATACGGTACAGGCTCGTCCAGTTCCTCCTGATCGTGTTTGGTACACT
GGTGATTGTTGCTCTCCTCGAGTTCACGCATTTCAGCTTCGTGGATATCT
TCACGAGTCTTCTTGCGTTCGTCCCAACCGGCTGGGGGATCTTGCTGATC
GCACAGGCTTTGAGGCCCGCGCTGCAGAAGATCGGGCTTATCTGGAACGC
GGTTATCTCCCTTGCTCGGTTGTATGACATACTGTTCGGGATAATCATCA
TGGTTCCCGTAGCGTTCATGTCGTGGATGCCTGGGTTTCAATCGATGCAA
ACGAGGATCTTATTCAATGAAGCTTTTAGCAGAGGGCTTCGTATCATGCA
GATTGTCACTGGGAAGAAATCAAAAGGCGATGTCGAAGTTGAAAAAAGAA
GGTAAAGCTTCTTATTTACCCAAACATATTTTATGTTCTGTTTGTTTGGA
ATCTTAAATTACAACAACACTAATGCAAAGCTTTTATAACTTGTGTAGGT
CTTGAGGTATATGGTAATTTAAAAGTTGCTGGTTTGCGGCGATGAGGTTA
GTTTTGTATTCTTATAAGTTATGCTTCTTGTCTGAATAAGAACTCAGACA
CCCGTGTTTTGTCTTCTTCTTATTTTAAACCAGGTTTTTGGAGAGCTTTG
GTTGAGGAAGCTATTTGATTAGTTCATCGGTTAGTGGGAGAAACATATAT
ATATACATTTGTCAGTACTTTGTTAGAGTGTGTGGAGGTGGGGACGACTC
TGATTCTGATTCCTTATGTGGTTATTGTCTGAAACGTTACAGCATTTTGT
GAAGTAGGCTTTTGTGCAAGATTTGATATCTTTCTCTCATTGTAGAGCTT
TAGAGCATTTTTTAGTATATGTTTAATCTTTGATTTTCTAATGTCATTTG
CATTCATATTCACATCTTCTCTGCTTCTTCGTTGTCTGAAACCAGTTTTC
AGATGCTTATCCATTGTCCA
SEQ ID NO. 7
TATCATTGTCGCAGTCTCTCTCACAGCTAAAAGGAAAACAGATCGCAACG
CTTCAATATCTCTCTCTCTCCATTGTTTTGAATTGAATCAGTCTCCTCAC
CCTTCTCCAAGATCCACCACACCACATGCCACTATGAGCCTCCGCCACCG
CACCGTCCCATCTCAAACCGGACGGCCGTCGGCGGCGGGAATCGAGGAGG
AGCCCTACAACATCATCCCCGTCAACAACCTCCTCGCCGACCATCCTTCT
CTCCGTTACCCCGAGGTCCGCGCCGCCGCCGCCGCTCTCAAAACCGTCGG
CGACCTCCGCCGTCCTCCCTACGTCCAATGGCGTCCTCACTACGATCTCC
TCGACTGGCTCGCCCTCTTCTTCGGCTTCCAGAAGGATAACGTCCGCAAC
CAGCGCGAGCACATGGTGCTCCACCTCGCCAACGCTCAGATGCGTCTCAC
GCCGCCGCCGGATAACATCGATTCCCTCGATTCGACGGTTGTCCGTCGCT
TTCGCCGGAAACTCCTCGGAAACTACTCGAGCTGGTGCTCTTATTTAGGG
AAGAAATCGAACATCTGGATCTCGGATCGGAACCCTGATTCGAGGCGGGA
GCTTCTCTACGTTGGCCTCTACCTCCTCGTGTGGGGCGAGGCGGCGAATC
TTAGGTTTATGCCTGAGTGCATCTGTTACATCTTCCATAACATGGCCTCG
GAGCTTAATAAGATTCTGGAAGATTGCCTCGATGAGAACACGGGGCAACC
CTATCTGCCTACTCTCTCGGGGGAAAACGCTTTCCTAAACGGCGTCGTTA
AACCTATTTACGAAACTATCAAAGCTGAGATTGATGAGAGCAAGAACGGG
ACGGAGCCGCATTGTAAGTGGAGGAACTATGATGATATTAATGAGTATTT
CTGGACGGATAGGTGTTTCAGTAAATTGAAGTGGCCGCTTGATCTGGGAA
GCAGTTTCTTCAAGAGTAGTAGAGGGAAAAGCGTTGGGAAAACCGGTTTT
GTGGAGCGCAGGACCTTCTTTTACCTCTACAGGAGCTTTGATAGGCTTTG
GGTGATGCTTGCGTTGTTCCTTCAGGCCGCCATTATAATTGCTTGGGAGG
AAAAGCCGGATAGAGGGTCGGTGACAGGGCAGCTGTGGAATGCCTTGAAG
TCCAGAGATGTCCAGGTGAGGCTTTTGACAGTTTTCTTGACGTGGAGTGG
GATGAGACTACTGCAGGCTGTGCTGGACGCTGGTTCGCAACGGTCTCTTA
TTTCTAGAGAGACCAAACGGCTCTTTTTCAGAATGTTGATGAAGGTTGTA
GCTGCCACAGTTTGGATAATAGCTTTTATTGTACTCTACACGAACATCTG
GAAGCAGAGGAAGCAAGACAGACAGTGGTCCAGAGCCGCGAATGACAAGA
TCTACCAGTTCCTTTACGCTGTGGTGGCCTTCTTGATCCCTGAGATCCTG
GCTCTGGCCCTGTTTATAATCCCGTGGATTAGGAACTTTCTGGAAGAGAG
CAATTGGAAAATATTCTTTGCTTTAACTTGGTGGTTCCAGGGTAAAAGCT
TTGTGGGTCGAGGTTTGAGAGAGGGTTTGGTGGACAACATCAAGTACTCG
ACGTTCTGGATCTTCGTCCTAGCAACAAAGTTCACATTCAGCTACTTCCT
ACAGGTTAAGCCAATGATTAAACCCTCGAAGCTGCTGTGGAACTTAAAGG
AGGTAGATTATGAGTGGCATCAGTTCTTTGGCGAGAGCAATAGGTTTTCT
GTCTTATTATTGTGGCTGCCAGTGGTGTTGATATACCTGATGGATATCCA
AATTTGGTACGCAATCTATTCTTCGATTGTTGGTGCTGTTGTTGGGCTGT
TTGATCATCTGGGGGAGATCAGGGACATGGGACAGCTTAGGCTGAGGTTT
CAGTTCTTTGCTAGCGCTATTCAGTTCAACCTGATGCCTGAGGAACAACT
CCTGAATGCTAGAGGCTTTGGTAACAAGCTTAAGGACGCCATCCATAGGT
AAGTCTGTGGAAGCATGTTACTGATTTCCTTTAGAGTTTAATGTACAGAG
TTTGTCTTTACGGTACAAGCTATATAATTTTAGTTTTGTTATAGCAGATT
CTCGTAACTTGACTTTTTAACATTGTTATAACCTTGAGTTTGTGTACTTT
ATGCAGATTGAAGCTGAGGTATGGATTTGGGCGGCCGTTTAAGAAACTCG
AGTCTAATCAGGTTGAGGCCAACAAGTTTGCTTTGATCTGGAATGAGATA
ATCTTAGCTTTTAGAGAAGAGGATATTGTCTCTGATCGAGAAGTAGAGCT
ACTGGAGCTGCCAAAGAATTCCTGGAATGTGACAGTTATCCGCTGGCCGT
GTTTCCTGCTGTGTAACGAGCTTTTGCTTGCACTGAGCCAGGCGAAAGAG
CTGGTTGATGCTCCTGATAAATGGCTGTGGCACAAGATATGCAAGAATGA
GTACAGGCGTTGTGCTGTGGTTGAGGCATACGACAGCATCAAACATCTGT
TGCTCTCGATCATCAAAATCGACACTGAAGAACATAAAATCATCACGGTT
TTCTTTCAGATGATTAAGGTTTCTATTCAGGGAGAGCAGTTCACCAAGAC
CTTCAAAGTGGATCTTCTGCCAAAGATTTACGAGACACTACAGAAGTTGG
TTGGGCTGTTGAATGGTGAGGAACCGGATATCGGGAGAGTGGTGAATGTT
CTGCAGTCTATATATGAGATTGCAACACGACAGTTCTTTATAGAAAAGAA
GACAACTGAACAGCTATCTACTGAAGGATTAACTCCTCATGATCCAGCCT
CAAAGTTACTGTTTCAGAACGCTGTTAGGCTTCCCGATGCAAGCAATGAA
GACTTCTTCCGGCAGGTGAGGCGGTTACACACAATTCTCACTTCTAGGGA
CTCTATGCACAGCGTCCCTGTGAATCTAGAGGCGAGACGTCGGATTGCCT
TCTTCAGCAATTCGCTCTTCATGAACTTGCCTCATGCACCTCAGGTGGAA
AAAATGTTGGCGTTCAGTGTTATGACTCCATACTACAGCGAGGAAGTTGT
ATACAGCAAAGAACAGCTCAAAAATGAGACTGAGGACGGGATTTCAACCT
TGTATTACCTGCAGACGATTTATGCTGACGAATGGAAAAATTTTAAGGAA
CGGATGCGTAGGGAAGGTATAAAGACAGATGATGAGCTGTATACAACCAA
GCTGAGAGAGCTCAGGCTTTGGGCTTCCTACAGAGGTCAGACTTTGGCAC
GTACAGTTCGAGGGATGATGTACTATTACAGGGCTCTTAAGATGCTTGCT
TTTCTTGACTCTGCGTCTGAAATGGACATTCGGGAGGATGCTCAGGAGCT
TGGTTCAATGAGGAGTTCGCAGGGAAATCTGGGCGGTCGATCGAATGGGG
TTGACGATGTAAATGACCGATCTTCTCTAAGCAGAGCTACTAGCTCCGTG
AGCATGCTGTATAAAGGCCATGAGCATGGGACTGCATTGATGAAATTCAC
ATATGTCGTGGCGTGCCAGATCTATGGGTCTCAAAAAGCGAAGAAGGAGC
CTCAGGCAGAGGAAATTCTCTATCTTATGAAGCAAAATGAAGCCCTCCGT
ATTGCGTATGTTGATGAGGTGCATGCAGGCAGGGGAGAGACCGAATATTA
CTCAGTTCTGGTGAAATACGATCACACTTTGGAGAAGGAAGTGGAGATAT
TCCGTGTGAAGCTACCTGGTCCCGTGAAGCTGGGTGAGGGAAAGCCAGAG
AACCAGAATCATGCAATGATCTTTACCCGTGGTGATGCTGTTCAGACCAT
AGATATGAACCAGGATAATTATTTTGAGGAGGCTCTCAAGATGAGAAATT
TGCTCCAGGAGTTTAGACATTATCATGGGATCAGAAAACCAACTATTCTT
GGTGTCCGAGAGCACATCTTCACTGGCTCTGTCTCGTCTCTGGCTTGGTT
CATGTCCGCTCAGGAGACTAGTTTCGTCACTCTTGGTCAGCGTGTTCTTG
CTAACCCGCTGAAGGTCAGAATGCATTATGGCCACCCCGATGTATTTGAC
AGATTCTGGTTCTTGAGTCGAGGTGGCATCAGCAAAGCGTCCAGAGTCAT
AAATATCAGTGAGGACATCTTCGCTGGGTTTAACTGCACATTGCGGGGCG
GTAACGTCACACACCACGAGTATATTCAGGTTGGTAACTATTCATTATTT
GGTTATTTCTAACTAATTTATACTGCGACAGCAATCCTATATTTCCATAT
TTCTTTTGTTATAACCATGCAACTTAACGTTCATTGTATCTGAGTGTCAA
TGTAAATTTCATAACCCCTGTTTTTGTGCATATATTCAGGTTGGGAAGGG
TCGAGATGTTGGATTGAATCAAATATCAATGTTTGAGGCGAAGGTAGCCA
GTGGGAACGGAGAGCAGGTTCTTAGCCGAGATGTGTACAGGCTGGGTCAT
AGACTCGATTTCTTCAGAATGTTATCATTTTTCTACACAACGGTCGGGTT
TTTCTTCAACACGATGATGGTCATTCTTACCGTTTACGCTTTCCTATGGG
GCCGGATTTATCTTGCTCTGAGCGGTGTTGAGAAGTCCGCTCTAGCAGAC
AGTACAGACACCAACGCAGCGCTTGCGGTTATATTGAACCAGCAGTTCAT
CATTCAGCTCGGTCTCTTCACAGCACTGCCAATGATTGTGGAATGGTCTC
TCGAGGAAGGTTTCCTTCTTGCGATATGGAATTTCATCCGGATGCAGATT
CAGCTTTCTTCTGTCTTCTACACATTCTCAATGGGGACCAGAGCTCACTA
TTTTGGCCGAACCATCCTCCACGGTGGAGCAAAGTACAGAGCCACTGGAC
GTGGATTTGTTGTCGAGCACAAGAGTTTCACTGAGAACTACAGACTGTAT
GCACGCAGTCACTTTGTGAAGGCCATCGAGCTTGGGCTGATCCTCATAGT
CTACGCTACGCACAGTCCCATCGCCAAAGACTCATTGATCTATATAGCCA
TGACTCTCACCAGCTGGTTCCTCGTGATATCATGGATACTGGCTCCATTT
GTGTTCAACCCGTCAGGTTTCGACTGGCTTAAGACGGTCTATGACTTCGA
AGGTTTCATGAACTGGATCTGGTATCAAGGCAGAATCTCAACGAAATCCG
AACAGAGCTGGGAGATATGGTGGTACGAGGAACAGGACCACCTGAGAACC
ACCGGTATACCAGGAAGAATAATGGAGATAATCTTGGACCTTCGGTTTTT
CTTCTTCCAGTACGGGATTGTATACCAGCTCAAAATCGCAAACGGATCAA
CCAGCATTCTCGTCTACTTGTTGTCGTGGATATACATCTTTGCTGTGTTT
GTGTTCTTCCTGGTAATCCAATACGCCCGTGACAAGTACTCTGCGAAAAA
CCACATACGGTACAGGCTTGTCCAGTTCCTCCTGATCGTGTTTGGTATTC
TGGTGATCGTTGCTCTGCTGGAGTTCACGCATTTCAGCTTCGTGGATATC
TTCACGAGTCTTCTTGCGTTCGTCCCAACCGGCTGGGGAATCTTGCTGAT
CGCACAGGCTTTAAGGCCTATGCTGCAGAAGTTCAGGCTTATCTGGAACG
CTGTTGTCTCCCTTGCTCGGTTATATGACATACTGTTGGGGATACTCATC
ATGGTTCCCGTAGCATTCATGTCATGGATGCCTGGGTTTCAGTCAATGCA
AACGAGGATCTTATTCAATGAAGCTTTTAGCAGAGGGCTTCGTATCATGC
AGATTGTCACTGGGAAGAAATCAAAAGGCGATGTCTAAGTTGAAAATGAA
AGAAGGTAAAGCTCTTTATTTACTCAAACATCTTTTATGTTCTGTTTGTT
TGGAATCCTAAGATTACAACAAAACCAATGCAAAGCTTTATAACTTGCGT
AGGTCATAATAAGGTACATGGTAATTTAAAGTTGTTGGTTTGCGGCGTTG
AGATCAGTTGGAGGTTAGTTTTCTCGAGACATGGTATTCTATAAGTTATT
GCTTCTTGTCTGAATAAGAACTCAAAAGAAAAACCATGTGTTGTTGTTGT
TGTCTTCTTCTTAAAGCAGGTGTTTGGAGAGCTTTTGTTGAGGAATCTGG
AAGTTGGGTTAGATCATGGGTTAGCGGGTGAAATATATATTGTCAGTACT
TTGTTAGTGTGTGGAAATGGGACTCTGATTCTTGTTGTCTGAAACGATAC
AGCATTTTGTGAAGTAGGGCTTTTGTGAAAGATTTGTTTCTCTTTCTCTC
TCAGTGTAGAGCTTTAGAGCATTTTTTAGTATATGTTTAATCTTTTTATT
TTCTAATGTCATTTGCATTCATATCTCACGTCTTCCTCGTTGGTCTGAAA
CCGGTTTTGAGATGCTTATCCATTGTCCACTTCTCTATG
SEQ ID NO. 8
CAAACGATCATAGAAATGTACCTGTAATTAGTCAACTGTTCAAAGAGCTT
AATGCATTGCTCCTTATCAATCTGCCCGGTTTTCTGTCATAAACATGAAA
TAGAATACAACGTAAACACATATGTTTAGCTATGTGGTAACTAACAGGGA
GCTTCCCTGTATATATCTGTAGGAGGTAAAAATCATTTTGAATGGAGTAA
CTTACGTCTTTGTCTATAAGACCAAAGGCTTTCTCCAGCATTCTTCTCTT
CATTTGATCCATTCCAGATACTTGCTTTGCGAGCTACAAAAATAACAAAT
ATGATACCACGCTAGTGCGGTCGTTTTTAGTTTTAACATACAAAAGATCA
AGTTTTGCAATAAACCTGTTCTTTGAAACTGTCATAAACAACGGCAAGAA
TCAAGTTCGTGACAAAGTAGACGCCAATTAGTACGTAGAGCACGAAGAAC
AACGAAGACCAGCGTGAAGACCTATACAAAAAAAAAAAAGAACAAATCAG
TCGCCCAAAACACTGGATGTAACTCATTTCACCATATAAACTTACTTGTA
AGCACGAATCCAGACATCAGGATTGTTGGATGTTGTGAACAAAATAAACA
TCTGGTAAAGAGTTGCACCGTAAGACGTGAAGACCGTGAGTCCTTGTTGT
GTATCCTCAAACATAACAAAAGCAATCCAACTGGCAAAGAGAAGGAACAG
CATCCATAGAGCCTGTGGAGATATTCAAGAAGTGAAAGCATAGAAGGACA
ATCAATACGAGTTCATCACTGTTCTGAAAGTCAAACTCGATTGGGAAGAC
CACTGACCAAGATATTCAAGTATGTGCCAAGCATTCCAGAAAGAAGGACG
AGGCTGTCTCGAAGTTCCCTGTACAGAAATTTTAAGAAACACAGTTTTTA
GACAAGAAGATAATATAAAACAAAAGTGCACTTTCATCGTAAATGGCATT
TTGGATTGAGACAAGTTGATAGAAAAGAAGCATAAATATGTGTTGATACC
TTATGCTGAGGATGAATATGATAACTCTGACATATGGGGCGATTCTAAAA
GGAAGAAAATCGAAAGCCACTGGAGACAAGTATAGAATGTCAACGAGCAC
ATCAACAATCAAAACTACCACGCAAGCAACCTGGAATCAGGAAGAAGAAG
ACATTTATTGTGTTAAAAGTTATGAAATCAATATCTTAAGTTCTGTCCAA
CATTCAACAAATAGAAAAGAAAAAAAAAAGAAACAGTGAAGAAGACTTAT
GTAGGGGAGAATACCTTCACTAGGTTAAGGCGGCTTGTCCAAAAGATACG
GGACCCTTCATAGGAGATTGGGAAAAAAGTATGTACAAGTAGTATAACCA
GGGTAAGTACCTGTTTGGAGAAAGCAAAAATATAAGAATTTCAGTACAAT
GAGAAAGGATCCTGGCTAGTCCGAACGATAAGATAACGAGTATATAAATA
TTAGCATCTACTTAAACGAGATCAACTAGTTTTCACAAAAGTGTGTAATG
AATCTCAAGCCTTGAACTTTCAAGTGGATGCATATAAGAATATGTCAGTA
CAGTGAGAAAGGATCCTGGCTAGTCTAGTCCCAACAATATACACCATGGA
AGATAAGACGAGTATGTTAATAAAATTAACAACTTCTTAAACGAGATCAA
CGAGTCTTGAATCTTTCAAAAGTGGGTTAATATGAATCTGGTGTGCCTAA
TTGCACTAAAATCTATTTGCATCTACAGCGAAAATGAAAGGCCAGCTACT
TGCCTCAAAGATAATGGATTCTGCATTGGTCAAGTAGGGTAACTCCCCAA
GGTAATAGTAATCTCTGTCTTTGCAAGACGGCGTAGGCTTATTTTCACAC
CACAATGGTTGCTGCAAAAAGACAGGACAAGAAAGACTGTCACACTTTTT
TCTATTTTCTTTTAAAAGTATTTTCTTTTGGGGGGGTTGGGGGGGGGGCA
AAAGGTTGCTGACCTCAAAGAAGTTAATCAAAATGAGAGCGAAATAGTTG
AGAGACCAGATGAGATCTAATCGTGTGTAGATAAAGTAGTACTTGGCAGA
CTCTCCGAAGCTGGACTGGTCAAGTATTTGCTCAGGTAGACCTATACCAT
CTTCAGCCTACATACATACAGCAAAATAAATAAATAAATAAATAAAAAGG
TTCTCAAAATCAGCAAAAAAGACAAAGAAACCGAACATTTTCTATTGCTA
TATTCGTAACTTATTATCAGGTTAAAGAAACAAATCATTTTGAACAAAAA
AAAAAAAAAAAGAAACAAATCATATTCTGAGCAATTATTCAGAATAAACG
ATTTGGACTTGTCAGACTGATTCTATTCACTGATAAATTGAGAAATCTCC
CTCAAGGGAGTTATGCCATCATGTCACAGTCAGTCGGACAAAGATCCATG
AATTTTATCAGAACAAGAAGAAGACAAATATAGTTAGGGAGTTGAGAGAT
AGAAAGAGAACCAGATCGACGAGAGCAGCAGCTTTCTGAAAGGTGGTACC
ATGAGCGATAGCTTCTGATCTACGAACAATCCGATCCGTACCACCACCAC
GACTCTCTCTACCTATCAAAGGATCTTCCATTTTTTTTTTCTTCAAAAGG
ATCGAAATGGAAAGCAGAAGACCAAGGACCTTATGTAGTACCTTTCTTTC
TTTCTTTCTGTCAGTTTATTGTTGGCTACGAATTTTAATATAATGATACA
GATCACAAGAAGAAGATAGTGATGATGGCCCAGAAGTAGTTGAATTTTTA
AAATAAATTAAATTTTCCAAGTGATATTAGATCTTGCCGACAAACACGTT
GGGGTCCCTTTCTCAATTTGTAACTTTGTGGATCAGGATCTAAGTAGATT
GGTCAAAACTGAGAGAGTGGTGTTGTGGTCCGAATAAAACTGATTAAAGA
ATAAGACTGCAGAGACTAACAAACAGAAGAAACAGATACATAGAGAAGTG
GACAATGGAGAAGCATCTCAAAACTGGTTTAACAAGCAGAGAAGAAGATG
TGAAATATGAATGCAAAATGACATTAGAAAATCAAAGATTAAACATATAC
TAAAAAGAAAGTACTCTAAAGCTCTACAATGAGAGAGAAAGAGAAGAAAT
CTGAACACAAAAGCCCTACTTCACACAAAATGCTGTAACGTTTCAGACAA
TAACACCATACAAGAATCAGAATCAGAGTCTTCCCCCCTTCCCCCACACT
CTAACAAAGTACTGACAAATGTATATATATATGTTTCCCCCACTAACCGA
TGCTCTAACCAGATAACCAACTTCCAAATTCGTCCACAAAAGCTCTCCAA
AAACCTGTTTCAAGCACAAGACAGAACAAAAGATGGGTTTATTTGAGTTC
TTCTTCGCACACCAAGGATAACTTAAAAAAAATTCCATCTCTCGAGAAAA
CTAACTAACCTCCAAATGATCACCATCGCCGCAAACCAACAGCTTTTTCA
AGTTACCATTTACCATACGACCTACACAAGTTATAAAGCTTCGCATTGGT
CTTGTTGTAAATCTTAGGATTCCAAAACAAACAGGATATTAAAGATGTTT
CAGTAAATAAAAGAGCTTTACCTTCTTCTTTCTTCAACTTAGACATCGCC
TTTTGATTTCTTCCCAGTGACAATCTGCATGATACGAAGCCCTCTGCTAA
AGGCTTCATTGAATAAGATCCTTGTTTGCATTGATTGAAACCCAGGCATC
CACGACATGAACGCTACGGGAACCATGATGAGTATCCCGAACAGTATGTC
ATATACCCTAGCAAGGGAGACAACCGCGTTCCAGATAAGCCTAAACTTCG
TCAGCGCTGGCCTTAAAGCCTGTGCGATCAGCAAGATTCCCCATCCGGTT
GGGACGAACGCAAGAAGACTCGTGAAGATATCCACGAAGCTGAAATGCGT
GAACTCTAGCAGAGCAACAATCACCAGAATACCAAACACGATCAGGAGGA
ACTGAACGAGCCTGTACCGTATGTGGTTTCTCGCAGAGTACTTGTCACGG
GCGTACTGGATTATCAGGAAGAACACAAACACTGCAAAGATGTATATCCA
TGACAACAAGTAGACGAGAATGCTGGTTGATCCGCTTGCGATTTTGAGCT
GGTAAACAATCCCGTACTGGAAGAAGAAAAACCGAAGGTCCAAGATTATC
TCCACAACTCTTCCTGGTATACCGGTGGTTCTCAGGTGGTCCTGTTCCTC
ATACCACCATATCTCCCAGCTCTGTTCCGATTTCGTTGAGATTCTGCCTT
GATACCAGATCCAGTTCATGAAGCCTTCGAAGTCATAGACCGTCTTAAGC
CAGTCGAATCCTGACGGGTTGAACACAAATGGGGCCAGTATCCATGATAT
CACGAGGAACCAACTGGTGAGAGTCATGGCTATGTAGATCAATGAGTCTT
TGGCGATGGGACTGTGCGTAGCATAGACTATGAGGATCAGCCCGAGCTCG
ATGGCCTTCACAAAGTGACTGCGTGCATAGAGTCGGTAGTTCTCGGTGAA
ACTCTTGTGCTCGACAACAAATCCACGTCCAGTGGCTCTGTACTTTGCTC
CACCATGGAGAATGGTTCGACCAAAATAGTGAGCTCTGGTCCCCATTGAG
AATGTGTAGAAGACAGAGGAAAGCTGAATCTGCATCCGAATGAAATTCCA
TATAGCTAGAAGGAAACCCTCCTCGAGAGACCATTCCACAATCATTGGCA
GGGCAGTGAACAGACCAAGCTGAACGATGAACTGCTGGTTCAGTATCACA
GCAAGCGCTGCGTTGGTGTCTGTACTGTCTGCTAGAGCGGACTTCTCAAC
ACCGCTCAGCGCGAGATAAACCCGGCCCCAGAGGAAAGCGTAAACCGTAA
GAATGACCATCATCGTGTTGAAGAAAAACCCGACCGTTGTGTAGAAAAAT
GATAACATTCTGAAGAAATCGAGCCTATGACCCAGCCTGTACACATCTCG
GCTAAGAACCTGCTCTCCATTCCCACTGGCTACCTTAGCCTCAAACATTG
ATATTTGATTCAATCCAACATCCCGGCCCTTCCCAACCTGAATATATGTA
CAAAAAACAAGGGTTATGCAATTTACATTGAAGAAACAAGGAACGTTAAG
TTGCATGGTTATAACAAAAAAAAAAATTGAACTATAGGATTTCTGTCGCA
GTATAAATTAGTTAGAAATATCAAAATAATGAACATTACCAACCTGAATA
TACTCGTGGTGGGTGACGTTACCGCCTCGCAATGTGCAATTAAACCCGGC
GAAGATGTCTTCACTGATATTTATGACTCTGGAAGCTTTGCTGATACCAC
CTCGACTCAAGAACCAGAATCTGTCAAAAACATCGGGGTGACCATAATGC
ATTCTGACCTTAAGCGGGTTGGCAAGAACACGCTGACCAAGAGTGACAAA
ACTAGTCTCCTGAGCAGACATGAACCACGCCAGAGACGAGACAGAGCCCG
TGAAGATGTGCTCCCGGACACCGAGAATAGTTGGCTTTCTGATCCCATGA
GATTTTCTAAACTCCTGGAGCAAATTTCTCATCTTGAGAGCCTCCTCAAA
ATAATTATCCTGGTTCATATCTATGGTCTGAACAGCATCACCACGGGTAA
AGATCATTGCATGATTCTGGTTCTCTGGCTTTCCCTCACCCAGCTTCAAC
GGACCAGGCAGCTTCACACGGAATATCTCCACTTCCTTCTCCAACGTGTG
ATCGTATTTCACCAGAACTGAGTAATACTCTGTCTCTCCCCTGCCCGCAT
GCACCTCATCCACATACGCAATACGGAGGGCTTCATTTTGCTTCATAAGA
TACAGAATTTCCTCTGCCTGAGGCTCCTTCTTTGCTTTTTGAGACCCATA
GATCTGGCACGCCACGACATATGTGAATTTCATCAATGCAGTCCCATGCT
CATGGCCTTTATACAGCATGCTCACGGAGCTAGTTGCTCTGCTTAGAGAA
GATCCACCATTTACATCGTCAACCCCATCCAGTCGACCATTTCCCTGCGA
ACTCCTCATTGAACCAAGCTCCTGAGCATCCTCCCGAATGTCCATTTCAG
ACGCAGAGTCAAGAAAAGCAAGCATCTTAAGAGCCCTGTAATAGTACATC
ATTCCTCGAACTGTACGTGCCAAAGTCTGACCTCTGTAGGAAGCCCAAAG
CCTGAGCTCTCTCAGCTTGGTTGTCCACAACTCAACATCTGTCTTTATAC
CTTCCCTACGCATCCGTTCCTTAAAATTTTTCCATTCGTCAGCATAAATC
GTCTGCAGGTAATACAAGGTTGAAATCCCATCCTCAGTCTCGTTTTTGAG
CTGTTCTTTGCTGTATACAACTTCCTCGCTGTAGTATGGAGTCAGAACAC
TGAACGCCAACATTTTCTCCACCTGAGGTGCATGAGGCAAGTTCATGAAG
AGCGAATTGCTGAAGAAGGCAATCCGCCGTCTCGCCTCTAGATTCACAGG
GACGCTGTGCATAGAGTCCCTAGAAGTGAGAATTGTGTGTAACCGCCTAA
CCTGCCGAAAGAAGTCTTCATTGCTTGCATCGGGAAGCCTAACAGCGTTC
TGAAACAGTAACTTGGAGGCTGGATCATGAGGAGTCAACCCCTCATTAGA
TAGCTGTTCAGTTGTCTTCTTCTCTATGAAGAACTGTCGTGTTGCAATCT
CATAAATCGACTGCAAAACATTCACCACTCGCTCAATATCTGGCTTCTCA
GCATTCAGTAGCCCAACCAACTTCTGAAGTGTCTCGTACATCTTTGGCAG
AAGGTCCACTTTAAAGGTCTTGGTGAACTGCTCACCCTGAATAGAGACCT
CAATCATCTGGAAGAAGATCGTAATGATTTTATGTTCTTCGGTGTCAATC
TTGATGATTGAGAGCAGCAGATGTTTGATGCTGTCATATGCCTCAACAAC
AGCACAACGCCTGTACTCATTCTTGCATATCTTGTGCCACAGCCATTTAT
CAGGAGCATCAACCAGCTCTTTCGCCTGGCTCAGTGCAAGTAAAAGCTCG
TTGCACAGCAGGAAACACGGCCAGCGGATAACTGACACATTCCACGAATT
CTTTGGCAGCTCCAGTAGCTCTACTTCTCGATCAGAGACTATATCCTCTT
CTCTGAAAGCTAAGATTATCTCATTCCAGATCAACGCAAACTTGTTGGCT
TCAACCTGATTAGACTCGAGTTTCTTAAACGGCCGCCCAAATCCATACCT
CAGCTTCAATCTGCATAAAGTACACAAACTCAAGGTTATTATTATGTTGA
AAACTCGAATTCCAAGCTTGTACCGTAAAGACAAACTCTGTATAGTAAAC
TATAAATATAGGAATATGCTTGCACAGACTTACCTATGAATGGCGTCCTT
AAGCTTGTTACCAAAGCCTCTAGCATTCAGGAGTTGTTCCTCAGGCATTA
GGTTGAACTGAATAGCGCTAGCAAAGAACTGAAACCTCAGCCTAAGCTGT
CCCATGTCCCTGATCTCCCCCAGATGATCAAACAACCCAACAATAGCACC
AACAATCGACGAATAGATTGCGTACCAAATTTGGATATCCATCAGGTATA
TCAACACCACTGGCAGCCATAATAACAAGACAGAAAACCTATTGCTCTCG
CCAAAGAACTGATGCCACTCATAATCTACCTCCTTTAAGTTCCATAGCAG
CTTCGAGGGTTTAATCATTGGTTTAACCTGCAGGAAGTAGCTGAAAATGA
ACTTCGTTGCTAGGACAAAGATCCAGAACGCCGAGTACTTGATGTTGTCC
ACCAAACCCTCTCTCAAACCTCGACCCACAAAGCTTTTACCCTGGAACCA
CCAAGTTAAAGCAAAGAATATTTTCCAATTGCTCTCTTCCAGAAAGTTCC
TTATCCACGGGATTATAAACAGAGCCAAAGCCAGGATCTCAGGGACCAAG
AATGCCACCACGGCGTAAAGGAATTGGTAGATCTTGTCATTCGCGGCTCT
GGACCACTGTCTATCTTGCTTCCTCTGCTTCCAGATGTTCGTGTAGAGAA
CAATAAAGGCTATAATCCAAACCGTGGCAGCTACAACCTTCATCAACATT
CTGAAAAACAGCCGTTTGGTCTCTCTAGAAATGAGGGGCCGTTGCGAACC
AGCGTCCAGCACAGCCTGCAGTAGTCTCATCCCACTCCACGTCAAGAAAA
CTGTCAAAAGCCTCACCTGAACATCTCTCGACTTCAAGGCATTCCACATC
TGCTTTGCCACCGACCCTCTATCCGGCTTCTCCTCCCAAGCAACTATAAT
GGCGGCTTGAAGGAACAAAGCAAGCATCACCCAAAGCCTATCAAAGCTCC
TGTAGAGATAAAAAAACGTCCTCCGCTCCACAAAACCGGTCTTCCCAACG
CTCTTCCCTCTACTATTCTTGAAGAAACTGCTCCCCAAATCAAGCGGCCA
TTTCAACTTACTGAAACACCTATCCGTCCAGAAATACTCATTAATATCAT
CATAGTTCCTCCACTTACAATGCGGCTCCGTCCCGTTCTTGCTCTCATTA
ATCTCAGCTCGGATCGTCTCGTAAATAGGTTTAACGACGCCGTTTAGGAA
AGCGTTCTCCCCAGAGAGAGTAGGCGTATAAGGCTGCCCCGTGTTCTCGT
CGAGGCAATCCTCCAACACCTTGTTAAGCTCCGAGGCCATATTGTGGAAG
ATGTAACAGACGCACTCGGGCATGAACCTAAGATTCGCGGCCTCGCCCCA
CACGAGGAGGTAGAGACCGACGTAGAGAAGCTCCCGCCTCGAATCGGGGC
TCCGATCCGAGATCCAGATGTTCGATTTCCTCCCGAGGTAGGAGCACCAG
CTGGAGTAGTTCCCGAGGAGCTTGCGGCGGAAGCGGCGGACGACGGCGGG
ATCGAGGGAGTCGATGTTGTCGGGGGGAGGGGAGAGGCGCATCTGGGCGT
TGGCGAGGTGGAGGACGACGTGCTCGCGCTGGTTGCGGACGTTGTCCTTC
TGGAAGCCGAAGAAGAGGGCGAGCCAGTCGAGGAGGTCGTAGTGAGGGCG
CCATTGGACGAAGGGAGGACGGCGGAGGTCTCCGACGGTTTTGAGAGCGG
CGGCGGCGGCGCGGACCTCGGGGTAGCGGAGGGAAGGGTGGTCGGCGAGG
AGGTTGTTGACGGGGATGATGTTGTAGGGCTCCTCCTCGGTTCCCCCCGC
CGACGGCCGTCCGGTTTGAGATGGGACGGTGCGGTGGCGGAGGCTCATAG
TAGCATGTGGTGGATCTTGGAGATGGTGTGAAGGAGGAGGAGACTGATTC
AAAGGATGTGGAGAGAGAGAGAGAGAGAGAGAGAGAGAGAGAGAGAGAGA
GAGATTGAATTGAAGCGTTGCGATATGTTTTTCTCTTTTAGCT

1.-10. (canceled)

11. (canceled)

12. A recombinant vector for editing a GSL5 gene in cruciferous crops, wherein a target sequence in the GSL5 gene for editing the GSL5 gene has the nucleotide sequence set forth in SEQ ID NO.9 or SEQ ID NO. 10.

13. A recombinant strain, comprising the recombinant vector of claim 12.

14. A method for improving resistance of cruciferous crops to clubroot disease, comprising knocking out a GSL5 gene in the cruciferous crops to lead to a function loss or alteration of the GSL5 gene, thus conferring an improved clubroot resistance to the cruciferous crops;
wherein the GSL5 gene in the cruciferous crops has the nucleotide sequence selected from the group consisting of SEQ ID NO.1, SEQ ID NO.2, SEQ ID NO.3, SEQ ID NO.4, SEQ ID NO.5, SEQ ID NO.6, SEQ ID NO.7, and SEQ ID NO.8; and
wherein the cruciferous crops include Brassica napus, Brassica rapa and Brassica oleracea.

15. The method according to claim 14, wherein said knocking out comprises gene-engineering of promoter region, coding region, and non-coding region of the GSL5 gene in the cruciferous crops.

16. The method according to claim 14, wherein said knocking out is implemented by gene editing, EMS mutagenesis, radiation mutagenesis, homologous recombination, or T-DNA insertion.

17. The method according to claim 14, wherein said knocking out is implemented by gene editing of the target sequence set forth in SEQ ID NO.9 or SEQ ID NO.10 within the GSL5 gene.
